(19) United States
(12) Patent Application Publication
(10) Pub. No.: US 2024/0238623 A9
(43) Pub. Date: Jul. 18, 2024

(54) Chimeric Antigen Receptor-Modified NK-92 Cells

(71) Applicant: ImmunityBio, Inc., Culver City, CA (US)

(72) Inventors: Laurent H. Boissel, Culver City, CA (US); Hans G. Klingemann, Culver City, CA (US)

(73) Assignee: ImmunityBio, Inc., Culver City, CA (US)

(21) Appl. No.: 17/812,264

(22) Filed: Jul. 13, 2022

Related U.S. Application Data
(63) Continuation of application No. 16/969,152, filed on Aug. 11, 2020, now Pat. No. 11,547,727.
(60) Provisional application No. 62/756,395, filed on Nov. 6, 2018.
(60) Provisional application No. 62/756,402, filed on Nov. 6, 2018.

(51) Int. Cl. A61K 35/17; A61K 35/28; A61K 39/395; A61K 45/06; A61P 35/00; C07K 14/705; C07K 14/735; C07K 16/10; C07K 16/28; C07K 16/32; C12N 5/0783

(52) U.S. Cl. CPC A61K 35/17; A61K 35/28; A61K 39/3955; A61K 45/06; A61P 35/00; C07K 14/70517; C07K 14/70521; C07K 14/70535; C07K 16/10; C07K 16/1018; C07K 16/1027; C07K 16/1063; C07K 16/109; C07K 16/2803; C07K 16/2827; C07K 16/2863; C07K 16/2866; C07K 16/2878; C07K 16/2887; C07K 16/32; C12N 5/0646; C07K 2317/53; C07K 2317/622; C07K 2319/03; C12N 2510/00

(57) ABSTRACT

Provided are NK-92 cells expressing a chimeric antigen receptor (CAR). The CAR can comprise an intracellular domain of FcϵRIγ. Also described are methods for treating a patient having or suspected of having a disease that is treatable with NK-92 cells, such as cancer or a viral infection, comprising administering to the patient NK-92-CAR cells.

This application is a continuation of U.S. patent application Ser. No. 16/969,152 filed on Aug. 11, 2020, which claims priority to U.S. provisional applications with the Ser. Nos. 62/756,395 and 62/756,402, both filed Nov. 5, 2018.

### SEQUENCE LISTING

The content of the XML file of the sequence listing named 104077.0003US2 Sequence Listing ST26, which is 150 kb in size was created on Jul. 13, 2022 and electronically submitted via EFS-Web along with the application is incorporated by reference in its entirety.

### FIELD OF THE INVENTION

The field of the invention is genetically modified immune competent cells that express a chimeric antigen receptor (CAR), and particularly modified NK-92 cells expressing a CAR with an Fc epsilon receptor gamma (FcϵRIγ) signaling domain.

### BACKGROUND OF THE INVENTION

Natural killer (NK) cells are cytotoxic lymphocytes that constitute a significant component of the innate immune system. In most cases, NK cells represent about 10-15% of circulating lymphocytes, and bind and kill targeted cells, including virus-infected cells and many malignant cells. NK cell killing is non-specific with regard to particular antigens and can occur without prior immune sensitization. Killing of targeted cells is typically mediated by cytolytic proteins, including perforin, granzyme, and granulysin.

Autologous NK cells have been used as therapeutic entities. To that end, NK cells are isolated from the peripheral blood lymphocyte fraction of whole blood, expanded in cell culture to obtain sufficient numbers of cells, and then re-infused into a subject. Autologous NK cells have shown in at least some cases moderate effectiveness in both ex vivo therapy and in vivo treatment. However, isolation and growth of autologous NK cell is time and cost intensive. Moreover, autologous NK cell therapy is further limited by the fact that not all NK cells are cytolytic.

At least some of these difficulties can be overcome by use of NK-92 cells, which are a cytolytic cancer cell line which was discovered in the blood of a subject suffering from a non-Hodgkins lymphoma and then immortalized in vitro (Gong et al., Leukemia 8:652-658 (1994)). While NK-92 cells are NK cell derivatives, NK-92 cells lack the major of inhibitory receptors that are otherwise displayed by normal NK cells, and retain the majority of the activating receptors. NK-92 cells do not, however, attack normal cells nor do they elicit an unacceptable immune rejection response in humans. Due to these desirable characteristics, NK-92 cells were characterized in detail and explored as therapeutic agent in the treatment of certain cancers as is described, for example, in WO 1998/049268 or US 2002/068044.

Phenotypic changes distinguishing a tumor cell from normal cells derived from the same tissue are often associated with one or more changes in the expression of specific gene products, including the loss of normal cell surface components or the gain of others (i.e., antigens not detectable in corresponding normal, non-cancerous tissue). The antigens which are expressed in neoplastic or tumor cells, but not in normal cells, or which are expressed in neoplastic cells at levels substantially above those found in normal cells, have been termed “tumor-specific antigens” or “tumor-associated antigens.” Such tumor-specific antigens may serve as markers for tumor phenotype. Tumor-specific antigens include cancer/testis-specific antigen (e.g. MAGE, BAGE, GAGE, PRAME and NY-ESO-1), melanocyte differentiation antigens (e.g. tyrosinase, Melan-A/MART, gp100, TRP-1 and TRP-2), mutated or aberrantly expressed antigens (e.g. MUM-1 , CDK4, beta-catenin, gp100-in4, p15 and N-acetylglucos-aminyltransferase V), and antigens that are expressed at higher levels in tumors (e.g., CD19 and CD20).

Tumor-specific antigens have been used as targets for cancer immunotherapies. One such therapy utilizes chimeric antigen receptors (CARs) expressed on the surface of immune cells, including T cells and NK cells, to improve cytotoxicity against cancer cells. CARs comprise a single-chain variable fragment (scFv) linked to at least one intracellular signaling domain. The scFv recognizes and binds an antigen on the target cell (e.g., a cancer cell) and triggers effector cell activation. The signaling domains contain immunoreceptor tyrosine-based activation domains (ITAMs) that are important for intracellular signaling by the receptor.

The first generation of CARs used in T-cells contained one cytoplasmic signaling domain. For example, one version of a first-generation CAR in T-cells included a signaling domain from the Fc epsilon receptor gamma (FcϵRIγ) which contained one ITAM, while another version contained the signaling domain from CD3ζ which contained three ITAMs. In vivo and in vitro studies showed that the CD3 CAR T-cells were more efficient at tumor eradication than FcϵRIγ CAR T-cells (e.g., Haynes, et al. 2001, J. Immunology 166:182-187; Cartellieri, et al. 2010, J. Biomed and Biotech, Vol. 2010, Article ID 956304). Additional studies then revealed that certain costimulatory signals were required for full activation and proliferation of such recombinant T-cells, and second and third generation CARs combined multiple signaling domains in to a single CAR to enhance efficacy of the recombinant CAR T-cells. Due to their less desirable philological effects in the tested T-cells, first generation CARs and the FcϵRIγ signaling domains were largely discarded in favor of the new, more efficient CARs using CD3ζ in combination with one or more additional signaling domains (e.g., Hermanson and Kaufman 2015, Frontiers in Immunol., Vol. 6, Article 195).

More recently, selected CARs have also been expressed in NK cells. For example, CAR-modified NK-92 cells have used first generation CARs with only a CD3ζ intracellular signaling domain. Several antigens have been targeted by these first generation CAR-NK cells, including CD19 and CD20 for B cell lymphoma, ErbB2 for breast, ovarian, and squamous cell carcinoma, GD2 for neuroblastoma, and CD138 for multiple myeloma. Second generation CAR-NK cells from the NK-92 line have also been created for several antigens, including EpCAM for multiple carcinomas HLA-A2 EBNA3 complex for Epstein—Barr virus, CS1 for multiple myeloma, and ErbB2 for HER2 positive epithelial cancers. The most common intracellular costimulatory domain used alongside CD3ζ in second generation NK-92 CARs is CD28. However, the potential effect of the CD28 domain is unclear since NK cells do not naturally express CD28. Additional second generation CARs have incorporated the 4-1BB intracellular signaling domain along with CD3ζ to improve NK cell persistence. Others compared functionality of different intracellular domains using an ErbB2 scFv fused with CD3ζ alone, CD28 and CD3ζ, or 4-1BB and CD3ζ tested against breast cancer cells. They found that both of the second generation constructs improved killing compared to the first generation CARs and the CD28 and CD3ζ had 65% target lysis, the 4-1BB and CD3ζ lysed 62%, and CD3ζ alone killed 51% of targets. 4-1BB and CD28 intracellular domains were also compared in a recent study using anti-CD19 CARs expressed on NK-92 cells for B cell malignances. Still others found that CD3ζ/4-1BB constructs were less effective than CD3ζ/CD28 in cell killing and cytokine production, highlighting differential effects of CD28 and 4-1BB costimulatory domains.

Third generation NK-92 CARs were constructed of an anti-CD5 scFv with CD3ζ, CD28, and 4-1BB intracellular signaling domains and demonstrated specific and potent anti-tumor activity against a variety of T-cell leukemia and lymphoma cell lines and primary tumor cells. Such cells were also able to inhibit disease progression in xenograft mouse models of T cell Acute lymphoblastic leukemia (ALL) cell lines as well as primary tumor cells (Transl Res. 2017 September; 187: 32-43). In further examples, WO 2016/201304 and WO 2018/076391 teach use of third generation CD3ζ CARs expressed in NK cells and NK-92 cells.

However, NK cells (and particularly NK-92 cells) are often difficult to genetically modify as evidenced by numerous failures to engineer NK-92 cells to express an Fc receptor. Such difficulties are further compounded where NK-92 cells are transfected with multiple recombinant genes or relatively large recombinant nucleic acid payload for heterologous expression. Additionally, NK-92 cells also exhibit a significant lack of predictability with respect to recombinant expression of exogenous proteins (e.g., CD16). On a functional level, while exhibiting in most cases targeted cytotoxicity, most if not all CAR NK-92 cells require a high effector to target cell ratio.

Therefore, even though numerous recombinant NK-92 cells are known in the art, all or almost all of them suffer from various difficulties. Consequently, there remains a need for CAR-expressing NK-92 cells that express a high-activity CAR in significant quantities, and that can be readily cultivated in a simple and effective manner.

### SUMMARY OF THE INVENTION

The inventors have discovered that NK-92 cells expressing an FcϵRIγ -containing CAR unexpectedly exhibit superior cytolytic activity, typically at a relatively low effector to target cell ratio as compared to other constructs, and high levels of expression of the FcϵRIγ-containing CAR. Moreover, such recombinant cells also expressed CD16 at desirable levels, and where further modified to express a stimulatory cytokine, recombinant NK-92 cells were also readily cultivated without the need for exogenous IL-2.

Therefore, in one aspect of the inventive subject matter, the inventors contemplate a genetically modified NK cell carrying a membrane bound recombinant chimeric antigen receptor (CAR) that comprises in a single polypeptide chain (i) an extracellular binding domain, (ii) a hinge domain, (iii) a transmembrane domain, and (iv) a FcϵRIγ signaling domain. Most typically, but not necessarily, the NK cell is an NK-92 cell.

In some embodiments, the extracellular binding domain comprises a scFv, which may specifically bind to a tumor-specific antigen (e.g., CD19, CD20, GD2, HER-2, CD30, EGFR, FAP, CD33, CD123, PD-L1, IGF1R, CSPG4, or B7-H4), a tumor associated antigen (e.g., MUC-2, brachyury, CEA), or a patient- and tumor-specific antigen (e.g., neoepitope with high affinity to the patient's MHC I and/or MHC II). Alternatively, the extracellular binding domain may also specifically bind to a virus-specific antigen, and typical viruses contemplated herein include an HIV virus, an HPV virus, an RSV virus, an influenza virus, an ebolavirus, or an HCV virus. For example, suitable viral antigens include gp120 of an HIV virus.

In further embodiments, the hinge domain and/or the transmembrane domain comprise a CD8 hinge domain and/or a CD28 transmembrane domain, and/or the FcϵRIγ signaling domain has an amino acid sequence of SEQ ID NO: 1.

Additionally, it is contemplated that the genetically modified NK cell may further carry a membrane bound recombinant CD16 (and especially a high-affinity variant of CD16), and/or the genetically modified NK cell may express a recombinant cytokine with an endoplasmic retention sequence.

Therefore, and viewed from a different perspective, the inventors also contemplate a genetically modified NK cell that comprises a recombinant nucleic acid encoding a chimeric antigen receptor (CAR), wherein the CAR includes in a single polypeptide chain (i) an extracellular binding domain, (ii) a hinge domain, (iii) a transmembrane domain, and (iv)a FcϵRIγ signaling domain. As noted before, it is generally preferred that the NK cell is an NK-92 cell. In some embodiments, the recombinant nucleic acid is an RNA, which may be a polycistronic RNA that further encodes a CD16 and/or a cytokine with an endoplasmic retention sequence. With respect to the various domains, the same considerations as noted above apply.

In a still further aspect of the inventive subject matter, the inventors also contemplate a method of treating cancer in a patient in need thereof that comprises a step of administering to the patient a therapeutically effective amount of the genetically modified NK cells presented herein, thereby treating the cancer. As will be readily appreciated, contemplated methods will further include a step of administering at least one additional therapeutic entity, including a viral cancer vaccine, a bacterial cancer vaccine, a yeast cancer vaccine, N-803, an antibody, a stem cell transplant, and/or a tumor targeted cytokine.

For example, cancers treated by contemplated methods include leukemia, acute lymphocytic leukemia, acute myelocytic leukemia, chronic leukemias, chronic myelocytic (granulocytic) leukemia, chronic lymphocytic leukemia, polycythemia vera, lymphomas, Hodgkin's disease, non-Hodgkin's disease, multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

Likewise, the inventors contemplate a method of treating a viral infection in a patient in need thereof that includes a step of administering to the patient a therapeutically effective amount of the genetically modified NK cells presented herein (having an extracellular binding domain may also specifically bind to a virus-specific antigen), thereby treating the viral infection. Of course, contemplated methods may further include a step of administering an antiviral drug.

Regardless of the type of treatment, it is contemplated that about 1×10⁸ to about 1×10¹¹ cells per m² of body surface area of the patient are administered to the patient.

Therefore, the inventors also contemplate use of genetically modified NK cells as presented herein in the treatment of cancer or a viral infection.

Various objects, features, aspects and advantages of the inventive subject matter will become more apparent from the following detailed description of preferred embodiments along with the accompanying drawing figures in which like numerals represent like components.

### BRIEF DESCRIPTION OF THE DRAWINGS

FIG. 1 is a schematic representation of exemplary CD19-CARs tested. All of the CD19-CAR variants contained an extracellular domain comprising an anti-CD19 scFv region (αCD19-scFv), a hinge region from CD8 (CD8 hinge), and a transmembrane domain from CD28 (CD28 TM ). The intracellular domains of the CD19CARs were varied as indicated.

FIG. 2A are exemplary results for the percentage of NK-92 cells expressing the CD19-CAR of FIG. 1 after transfection with CD19-CAR mRNA as determined by flow cytometry with an anti-scFv antibody labeled with eF660.

FIG. 2B are exemplary results for the median fluorescent intensity (MFI) minus background for CD19-CAR-expressing NK-92 cells labeled with an anti-scFv antibody labeled with eF660.

FIG. 3A shows exemplary results for the percentage of NK-92 cell-sensitive target cancer cells (K562) that were killed by NK-92 cells (effector) expressing the CD19CARs at effector:target ratios of from 5:1 to 0.3:1.

FIG. 3B shows exemplary results for the percentage of NK-92 cell-resistant, CD19-positive target cancer cells (SUP-B15) that were killed by NK-92 cells (effector) expressing the CD19CARs at effector:target ratios of from 5:1 to 0.3:1.

FIG. 4 shows exemplary results for the MFI of CD19-CAR-expressing NK-92 cells (effector) labeled with anti-CD107a antibody in a degranulation assay with SUP-B15 target cells at effector:target ratios of from 2:1 to 0.25:1.

FIG. 5 shows an exemplary survival curve of IV Raji tumor bearing animals, as described in the Examples. Statistical analysis was Log-rank (Mantel-Cox) test. ****, P<0.0001.

FIG. 6 shows exemplary results for animal body weight change in the IV Raji tumor model. Data are mean±SEM. SEM was calculated as Standard Deviation divided by the square root of N.

FIG. 7 shows an exemplary tumor growth curve for the SC Raji model. Data are Mean±SEM. Statistical analyses were done using 2-way ANOVA followed by multiple comparison by Tukey test; ***, P<0.001; ****, P<0.0001.

FIG. 8 shows exemplary data indicating CD19 t-haNK reduced metastatic disease burden in the livers of SC Raji tumor-bearing mice. Panel a: Whole liver images of animals from indicated treatment groups on Day 13. Arrows indicate metastatic lesions. Livers were fixed in 10% formalin for at least 24 hours prior to photography. Panel b: Quantification of percentage involvement of tumor cells in the liver (evaluated by H&E staining) on indicated days. On Day 13: *, P=0.0257 by unpaired 2-tailed t test. Statistical analyses for Days 11 and 15 could not be performed due to limited sample size. See Table 4 for raw data.

FIG. 9 shows exemplary results for animal body weight change in the SC Raji tumor model. Data are mean±SEM.

FIG. 10 shows an exemplary Kaplan-Meier survival curve of mice injected with L1210-Luc tumor cells following intratumoral treatment with mCD19-CAR NK-92 cells vs. vehicle control, as described in the Examples.

FIG. 11 shows exemplary results for tumor size of complete responders vs. naive controls re-challenged with L1210-Luc tumor cells, as described in the Examples.

FIG. 12 shows an exemplary Kaplan-Meier survival curve of mice injected with A20 tumor cells following intratumoral treatment with mCD19-CAR NK-92 cells vs. vehicle control, as described in the Examples.

FIG. 13 shows exemplary results for tumor size of complete responders vs. naive controls re-challenged with A20 tumor cells, as described in the Examples.

FIG. 14 shows exemplary results for cytotoxicity of HER2.CAR-t-haNK cells against BT-474 cells.

FIG. 15 shows exemplary results for cytotoxicity of CD33.CAR-t-haNK cells against THP-1 cells.

FIG. 16 shows exemplary results for cytotoxicity of PD-L1.CAR-t-haNK cells against SUP-B15.PD-L1+cells.

FIG. 17 shows exemplary results for cytotoxicity of PD-L1.CAR-t-haNK cells against U251 cells.

FIG. 18 shows exemplary results for cytotoxicity of EGFR.CAR-t-haNK cells against A-549 cells.

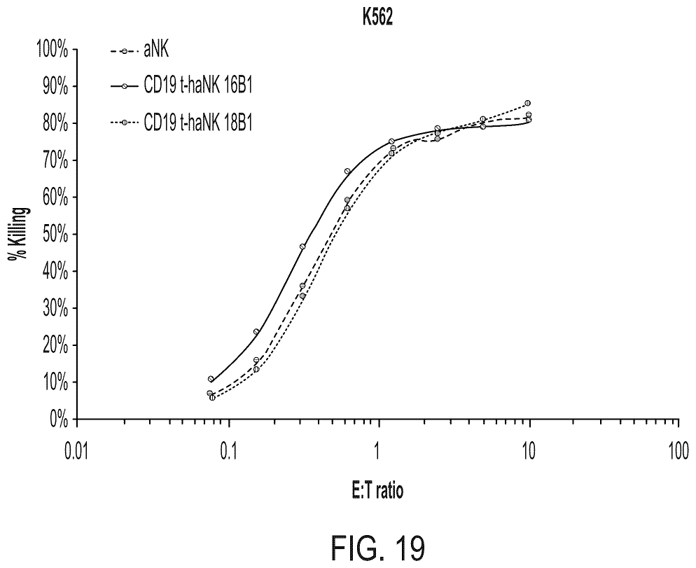

FIG. 19 shows exemplary results for cytotoxicity of CD19.CAR-t-haNK cells against K562 cells.

FIG. 20 shows exemplary results for cytotoxicity of CD19.CAR-t-haNK cells against SUP-B15 cells.

FIG. 21 shows exemplary results for ADCC of CD19.CAR-t-haNK cells against SKBr3 cells.

FIG. 22 shows exemplary results for cytotoxicity of IGF1R.CAR-t-haNK cells against MDA-MB-231 cells.

FIG. 23 shows exemplary results for cytotoxicity of PD-L1.CAR-t-haNK cells against a variety of cancer cells.

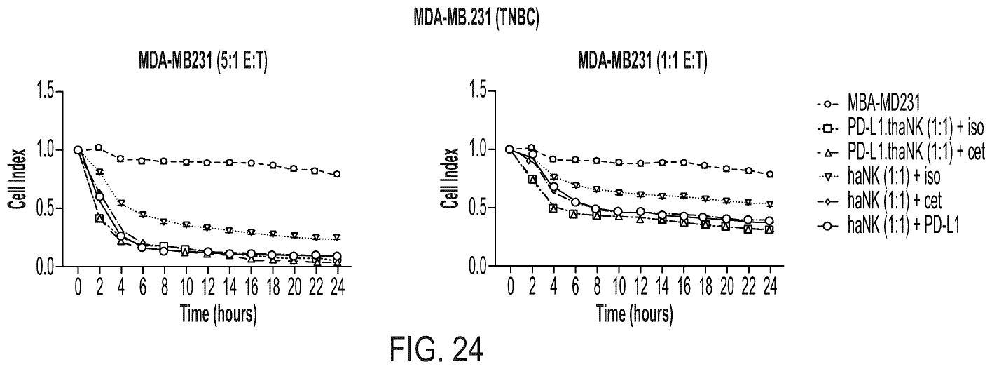

FIG. 24 shows exemplary comparative results for cytotoxicity of PD-L.CAR-t-haNK cells against MDA-MB-231 cells.

FIG. 25 shows exemplary results expression of CD16 and CD19.CAR.

FIG. 26 shows exemplary results for natural cytotoxicity of CD19.CAR-t-haNK cells.

FIG. 27 shows exemplary results for CAR mediated cytotoxicity of CD19.CAR-t-haNK cells.

FIG. 28 shows exemplary results for ADCC of CD19.CAR-t-haNK cells.

FIG. 29 shows exemplary comparative results for expression of CD16 and CD20.CAR.

FIG. 30 shows exemplary results for natural cytotoxicity of CD20.CAR-t-haNK cells.

FIG. 31 shows exemplary results for expression of CD16 and CD33.CAR.

FIG. 32 shows exemplary results for natural cytotoxicity of CD33.CAR-t-haNK cells.

FIG. 33 shows exemplary results for CAR mediated cytotoxicity of CD33.CAR-t-haNK cells.

FIG. 34 shows exemplary results for ADCC of CD33.CAR-t-haNK cells.

FIG. 35 shows exemplary results for expression of CD16 and EGFR.CAR.

FIG. 36 shows exemplary results for natural cytotoxicity of EGFR.CAR-t-haNK cells.

FIG. 37 shows exemplary results for CAR mediated cytotoxicity of EGFR.CAR-t-haNK cells.

FIG. 38 shows exemplary results for CAR mediated cytotoxicity of EGFR.CAR-t-haNK cells.

FIG. 39 shows exemplary results for ADCC of EGFR.CAR-t-haNK cells.

FIG. 40 shows exemplary results for expression of CD16 and HER2.CAR.

FIG. 41 shows exemplary results for natural cytotoxicity of HER2.CAR-t-haNK cells.

FIG. 42 shows exemplary results for CAR mediated cytotoxicity of HER2.CAR-t-haNK cells.

FIG. 43 shows exemplary results for ADCC of HER2.CAR-t-haNK cells.

FIG. 44 shows exemplary results expression of CD16 and PD-L1.CAR.

FIG. 45 shows exemplary results for natural cytotoxicity of PD-L1.CAR-t-haNK cells.

FIG. 46 shows exemplary results for CAR mediated cytotoxicity of PD-L1.CAR-t-haNK cells.

FIG. 47 shows exemplary results for ADCC of PD-L1.CAR-t-haNK cells.

FIG. 48 shows exemplary results for CAR mediated cytotoxicity of CD123.CAR-t-haNK cells.

FIG. 49 shows exemplary results for ADCC of CD123.CAR-t-haNK cells.

FIG. 50 shows exemplary results for expression of CD16 and CD30.CAR.

FIG. 51 shows exemplary results for natural cytotoxicity of CD30.CAR-t-haNK cells.

FIG. 52 shows exemplary results for CAR mediated cytotoxicity of CD30.CAR-t-haNK cells.

FIG. 53 shows exemplary results for ADCC of CD30.CAR-t-haNK cells.

FIG. 54 shows exemplary results for CD16 and BCMA.CAR expression.

FIG. 55 shows exemplary results for CAR mediated cytotoxicity of BCMA.CAR-t-haNK cells.

FIG. 56 shows exemplary results for ADCC of BCMA.CAR-t-haNK cells.

FIG. 57 shows exemplary results for expression of CD16 and gp120.CAR.

FIG. 58 shows exemplary results for GP120 binding of gp120.CAR-t-haNK cells.

FIG. 59 shows exemplary results for natural cytotoxicity of gp120.CAR-t-haNK cells.

FIG. 60 shows exemplary results for ADCC of gp120.CAR-t-haNK cells.

FIG. 61 shows exemplary results for CD16 and FAP.CAR expression.

FIG. 62 shows exemplary results for CAR mediated cytotoxicity of FAP.CAR-t-haNK cells.

FIG. 63 shows exemplary results for CSPG4 expression in CSPG4.CAR-t-haNK cells.

FIG. 64 shows exemplary results for CAR mediated cytotoxicity of CSPG4.CAR-t-haNK cells.

FIG. 65 depicts an exemplary tricistronic construct encoding IGF1R-CAR, CD16, and IL-2^{ER}.

### DETAILED DESCRIPTION OF THE INVENTION

To date, FcϵRIγ -containing CARs have not been utilized in NK-92 cells, other NK cell lines, or endogenous NK cells because as signaling domains (e.g., CD3ζ) were deemed more efficient, especially when combined with additional signaling domains (in second and third generation CARs). The inventors have now made the unexpected and surprising finding that NK-92 cells expressing a first-generation CAR comprising an intracellular domain from FcϵRIγ , which has only one ITAM domain, have equal or higher cytotoxic activity against cancer cells expressing the antigen recognized by the CAR than NK-92 cells expressing CARs with a CD3ζ signaling domain, which has three ITAM domains, even where these ITAM domains were combined with other signaling domains (i.e., second or third generation CARs). Notably, the IgE receptor (FcϵRI) in its native context includes two gamma chains coupled to each other via a disulfide bond and is normally expressed only in eosinophils, basophils, and epidermal Langerhans cells. The inventors also made the unexpected finding that a CAR comprising an intracellular domain from FcϵRIγ was expressed at higher levels on the surface of NK-92 cells than other CARs, especially those comprising the CD3ζ signaling domain.

Therefore, the inventive subject matter is directed to a genetically modified NK-92 cell or NK cell line engineered to express a chimeric antigen receptor (CAR) on a cell surface. Most typically, the CAR comprises an intracellular domain from the Fc epsilon receptor gamma (FcϵRIγ ), however, in other embodiments the CAR may also comprise a T cell receptor (TCR) CD3 zeta (CD3ζ) intracellular domain. As will be readily appreciated, the CAR may be transiently or stably expressed by the NK-92 cell from a recombinant DNA or RNA molecule.

Consequently, in one aspect of the inventive subject matter, an NK cell, an NK-92 cell or NK/NK-92 cell line expresses a chimeric antigen receptor (CAR) on the surface of the NK-92 cell that comprises a cytoplasmic domain of FcϵRIγ (e.g., having amino acid sequence of SEQ ID NO:1). Alternatively, or additionally, the CAR may also comprise a cytoplasmic domain of CD3 zeta (e.g., having amino acid sequence of SEQ ID NO: 10, which may be encoded by a nucleic acid of SEQ ID NO:11 (codon optimized) or SEQ ID NO:12 (non-codon-optimized); full-length sequence is shown in SEQ ID NO:47). In another aspect, an NK or NK-92 cell line is contemplated that is transformed with a nucleic acid encoding a chimeric antigen receptor (CAR). For example, preferred nucleic acids encode a cytoplasmic domain of FcϵRIγ (e.g., comprising or consisting of SEQ ID NO:2). Alternatively, or additionally, the nucleic acid encodes a cytoplasmic domain of CD3 zeta (e.g., comprising or consisting of SEQ ID NO:11 (human, codon optimized) or SEQ ID NO:12 (human)). As will be readily appreciated, the CAR may target a cancer-associated or a virus-associated antigen via its extracellular binding domain as is described in more detail below.

In further contemplated embodiments, the NK or NK-92 cell can be modified to express at least one cytokine or variant thereof. For example, the cytokine may be transiently or stably expressed by the recombinant cell, and the cytokine may include an endoplasmic retention signal. Where desired, the NK or NK-92 cell may also be modified to express a suicide gene (e.g., suicide gene is thymidine kinase). Without being bound by any theory, it is believed that expression of a suicide gene can prevent uncontrolled proliferation of the NK-92 cells by providing a mechanism for selectively killing the cells upon introduction of a suitable stimulus.

In another aspect of the inventive subject matter, the inventors also contemplate a method of treating cancer in a patient in need thereof that includes a step of administering to the patient a therapeutically effective amount of modified NK/NK-92 cells or an NK/NK-92 cell line engineered to express a chimeric antigen receptor (CAR) as described herein. Viewed form a different perspective, the inventors also contemplate a modified NK/NK-92 cell or a NK/NK-92 cell line that expresses a chimeric antigen receptor (CAR), preferably comprising a cytoplasmic domain of FcϵRIγ , for use in treating a tumor in a subject. In some embodiments, the use comprises administering to the subject an effective amount of modified cells or the cell line described herein to treat the tumor. In yet other embodiments, an in vitro method for killing tumor cells is contemplated and may include a step of contacting a tumor cell with a modified NK-92 cell or NK-92 cell line described herein. In some embodiments, the modified NK-92 cell or NK-92 cell line expresses a CAR that binds to an antigen on the tumor cell. In some embodiments, the CAR preferably comprises an intracellular domain from the Fc epsilon receptor gamma (FcϵRIγ ). Alternatively, or additionally, the CAR comprises a T cell receptor (TCR) CD3 zeta (CD3ζ) intracellular domain.

In still other embodiments, a method of treating a viral infection in a patient in need thereof is described, the method comprising administering to the patient a therapeutically effective amount of CAR-expressing NK-92 cells as described herein.

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

Titles or subtitles may be used in the specification for the convenience of a reader, which are not intended to influence the scope of the present invention. Additionally, some terms used in this specification are more specifically defined below.

With respect to suitable NK cells, it should be noted that all NK cells are deemed suitable for use herein and therefore include primary NK cells (preserved, expanded, and/or fresh cells), secondary NK cells that have been immortalized, autologous or heterologous NK cells (banked, preserved, fresh, etc.), and modified NK cells as described in more detail below. In some embodiments, it is preferred that the NK cells are NK-92 cells. The NK-92 cell line is a unique cell line that was discovered to proliferate in the presence of interleukin 2 (IL-2) (see e.g., Gong et al., Leukemia 8:652-658 (1994)). NK-92 cells are cancerous NK cells with broad anti-tumor cytotoxicity and predictable yield after expansion in suitable culture media. Advantageously, NK-92 cells have high cytolytic activity against a variety of cancers.

The original NK-92 cell line expressed the CD56^{bright}, CD2, CD7, CD11a, CD28, CD45, and CD54 surface markers and did not display the CD1, CD3, CD4, CD5, CD8, CD10, CD14, CD16, CD19, CD20, CD23, and CD34 markers. Growth of such NK-92 cells in culture is dependent upon the presence of interleukin 2 (e.g., rIL-2), with a dose as low as 1 IU/mL being sufficient to maintain proliferation. IL-7 and IL-12 do not support long-term growth, nor have various other cytokines tested, including IL-1α, IL-6, tumor necrosis factor α, interferon α, and interferon γ. Compared to primary NK cells, NK-92 typically have a high cytotoxicity even at relatively low effector:target (E:T) ratios, e.g. 1:1. Representative NK-92 cells are deposited with the American Type Culture Collection (ATCC), designation CRL-2407.

Therefore, suitable NK cells may have one or more modified KIR that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest (see URL www.nantkwest.com) as aNK cells (activated natural killer cells). Such cells may then be additionally genetically modified to a CAR as further described in more detail below.

In another aspect of the inventive subject matter, the genetically engineered NK cell may also be an NK-92 derivative that is modified to express the high-affinity Fcγ receptor (CD16). Sequences for high-affinity variants of the Fcy receptor are well known in the art (see e.g., Blood 2009 113:3716-3725; SEQ ID NO:43 and 44), and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells. Such cells may then be additionally genetically modified to a CAR as further described in more detail below.

Therefore, NK cells suitable for use herein include NK-92 cells (which may be transfected with a tricistronic construct encoding a CAR, a CD16 or variant thereof, and a cytokine or variant thereof), a genetically modified NK cell or NK-92 cell that expresses a CD16 or variant thereof or a cytokine or variant thereof (which may be transfected with a nucleic acid encoding a CAR and a CD16 or variant thereof or a cytokine or variant thereof), and a genetically modified NK cell or NK-92 cell that expresses a CD16 or variant thereof and a cytokine or variant thereof (which may be transfected with a nucleic acid encoding a CAR)

Genetic modification of the NK cells contemplated herein can be performed in numerous manners, and all known manners are deemed suitable for use hereon. Moreover, it should be recognized that NK cells can be transfected with DNA or RNA, and the particular choice of transfection will at least in part depend on the type of desired recombinant cell and transfection efficiency. For example, where it is desired that NK cells are stably transfected, linearized DNA may be introduced into the cells for integration into the genome. On the other hand, where transient transfection is desired, circular DNA or linear RNA (e.g. ,mRNA with polyA⁺ tail) may be used.

Similarly, it should be appreciated that the manner of transfection will at least in part depend on the type of nucleic acid employed. Therefore, viral transfection, chemical transfection, mechanical transfection methods are all deemed suitable for use herein. For example, in one embodiment, the vectors described herein are transient expression vectors. Exogenous transgenes introduced using such vectors are not integrated in the nuclear genome of the cell; therefore, in the absence of vector replication, the foreign transgenes will be degraded or diluted over time.

In another embodiment, the vectors described herein allow for stable transfection of cells. In one embodiment, the vector allows incorporation of the transgene(s) into the genome of the cell. Preferably, such vectors have a positive selection marker and suitable positive selection markers include any genes that allow the cell to grow under conditions that would kill a cell not expressing the gene. Non-limiting examples include antibiotic resistance, e.g. geneticin (Neo gene from Tn5).

Alternatively, or additionally, the vector is a plasmid vector. In one embodiment, the vector is a viral vector. As would be understood by one of skill in the art, any suitable vector can be used, and suitable vectors are well-known in the art.

In still other embodiments, the cells are transfected with mRNA encoding the protein of interest (e.g., the CAR). Transfection of mRNA results in transient expression of the protein. In one embodiment, transfection of mRNA into NK-92 cells is performed immediately prior to administration of the cells. In one embodiment, “immediately prior” to administration of the cells refers to between about 15 minutes and about 48 hours prior to administration. Preferably, mRNA transfection is performed about 5 hours to about 24 hours prior to administration. In at least some embodiments as described in more detail below, NK cell transfection with mRNA resulted in unexpectedly consistent and strong expression of the CAR at a high faction of transfected cells. Moreover, such transfected cells also exhibited a high specific cytotoxicity at comparably low effector to target cell ratios.

With respect to contemplated CARs it is noted that the NK/NK-92 cells will be genetically modified to express the CAR as a membrane bound protein exposing a portion of the CAR on the cell surface while maintaining the signaling domain in the intracellular space. Most typically, the CAR will include at least the following elements (in order): an extracellular binding domain, a hinge domain, a transmembrane domain, and a FcϵRIγ signaling domain.

In preferred embodiments, the cytoplasmic domain of the CAR comprises or consists of a signaling domain of FcϵRIγ . For example, the FcϵRIγ signaling domain comprises or consists of or consists essentially of the amino acid sequence of SEQ ID NO:1. In some embodiments, the FcϵRIγ cytoplasmic domain is the sole signaling domain. However, it should be appreciated that additional elements may also be included, such as other signaling domains (e.g., CD28 signaling domain, CD3ζ signaling domain, 4-1BB signaling domain, etc.). These additional signaling domains may be positioned downstream of the FcϵRIγ cytoplasmic domain and/or upstream of the FcϵRIγ cytoplasmic domain.

In some embodiments, the FcϵRIγ signaling domain comprises or consists of or consists essentially of an amino acid sequence having at least about 85%, 90%, 95%, 96%, 97%, 98%, or 99% sequence homology to the amino acid sequence of SEQ ID NO:1.

As noted above, in some embodiments, the cytoplasmic domain of the CAR comprises a signaling domain of CD3 zeta (CD3ζ). In one embodiment, the cytoplasmic domain of the CAR consists of a signaling domain of CD3 zeta. In one embodiment, the CD3 zeta signaling domain comprises or consists of or consists essentially of the amino acid sequence of SEQ ID NO:10. In some embodiments, the CD3 zeta signaling domain comprises or consists of or consists essentially of an amino acid sequence having at least about 85%, 90%, 95%, 96%, 97%, 98%, or 99% sequence homology to the amino acid sequence of SEQ ID NO:10.

The CAR may comprise any suitable transmembrane domain. In one aspect, the CAR comprises a transmembrane domain of CD28. In one embodiment, the CD28 transmembrane domain comprises or consists of or consists essentially of the amino acid sequence of SEQ ID NO:7. In one embodiment, the CD28 transmembrane domain comprises or consists of or consists essentially of an amino acid sequence having at least about 85%, 90%, 95%, 96%, 97%, 98%, or 99% sequence homology to the amino acid sequence of SEQ ID NO:7. In one embodiment, the transmembrane domain is selected from a CD28 transmembrane domain, 4-1BB transmembrane domain, or FcϵRIγ transmembrane domain.

The CAR may comprise any suitable hinge region. In one aspect, the CAR comprises a hinge region of CD8. In one embodiment, the CD8 hinge region comprises or consists of or consists essentially of the amino acid sequence of SEQ ID NO:6. In one embodiment, the CD8 hinge region comprises or consists of or consists essentially of an amino acid sequence having at least about 85%, 90%, 95%, 96%, 97%, 98%, or 99% sequence homology to the amino acid sequence of SEQ ID NO:6.

Most typically, but not necessarily, the extracellular binding domain of the CAR will be a scFv or other natural or synthetic binding portion that specifically binds an antigen of interest. Especially suitable binding portions include small antibody fragments with single, dual, or multiple target specificities, beta barrel domain binders, page display fusion proteins, etc. Among other suitable extracellular binding domains, preferred domains will specifically bind to a tumor-specific antigen, a tumor associated antigen, or a patient- and tumor-specific antigen. For example, contemplated antigens include CD19, CD20, GD2, HER-2, CD30, EGFR, FAP, CD33, CD123, PD-L1, IGF1R, CSPG4, or B7-H4. Further tumor-specific antigens are described, by way of non-limiting example, in US2013/0189268; WO 1999024566 A1; U.S. Pat. No. 7,098,008; and WO 2000020460, each of which is incorporated herein by reference in its entirety. Likewise, other preferred domains will specifically bind to a (pathogenic) virus-specific antigen, such as an antigen of an HIV virus (e.g., gp120), an HPV virus, an RSV virus, an influenza virus, an ebolavirus, or an HCV virus.

With respect to the construction of contemplated CARs it should be recognized that CARs can be engineered in numerous manners as described, for example, in WO 2014/039523; US 2014/0242701; US 2014/0274909; US 2013/0280285 and WO 2014/099671, each of which is incorporated herein by reference in its entirety.

Therefore, and viewed from a different perspective, contemplated CARs target an antigen associated with a specific cancer type. In one embodiment, the cancer is leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, or retinoblastoma.

Therefore, contemplated CARs will generally have a structure of an extracellular binding domain that is (directly) coupled to a hinge domain, which is (directly) coupled to a transmembrane domain, which is (directly) coupled to an FcϵRIγ signaling domain. In still further contemplated aspects, contemplated CARs may also include one or more signaling domains in addition to or replacing the FcϵRIγ signaling domain, and especially contemplated signaling domains include CD3ζ signaling domains, 4-1BB signaling domains, and CD28 signaling domains. For example, contemplated CARs may therefore include any one of the binding domains having SEQ ID NO:4, 23-42, and 48-59 that is coupled to a hinge domain (e.g., CD8 hinge as in SEQ ID NO:6), which is in turn coupled to a transmembrane domain (e.g., CD28 TM as in SEQ ID NO:7), which is coupled to a signaling domain (e.g., FcϵRIγ signaling domain as in SEQ ID NO:1, CD28 signaling domain as in SEQ ID NO:8, 4-1BB signaling domain as in SEQ ID NO:9, CD3ζ signaling domain as in SEQ ID NO:10)

In still further contemplated aspects, NK cells may be further genetically modified to express one or more cytokines to so provide a selection marker where the cytokine and the CAR are encoded on the same recombinant nucleic acid and/or to render the recombinant cells independent of exogenous IL-2. Therefore, in some embodiments, NK-92 cells are modified to express at least one cytokine. In particular, the at least one cytokine is IL-2, IL-12, IL-15, IL-18, IL-21, or a variant thereof. In preferred embodiments, the cytokine is IL-2 or a variant thereof and especially preferred variants include endoplasmic retention signals (e.g., human IL-2 as in SEQ ID NO:18, or with ER retention signal as in SEQ ID NO:19). For example, the IL-2 gene is cloned and expressed with a signal sequence that directs the IL-2 to the endoplasmic reticulum. This permits expression of IL-2 at levels sufficient for autocrine activation, but without releasing IL-2 extracellularly (e.g., Exp Hematol. 2005 Feb; 33(2):159-64.) Alternatively, expression of a cytokine (and especially IL-15) may also be such that the cytokine will be expressed in sufficient quantities to provide an autocrine growth signal to the recombinant cells, but also to allow at least some of the expressed IL-15 to be released from the cell, which will so provide an immune stimulatory signal. For example, such expression may be achieved using a human IL-15 sequence that includes both the signal peptide and an endoplasmic retention sequence. An exemplary DNA and protein sequence for an endoplasmic retained IL-15 is shown in SEQ ID NO:72 and SEQ ID NO:73, respectively.

Where desired, contemplated cells may also express a suicide gene. The term “suicide gene” refers to a transgene that allows for the negative selection of cells expressing the suicide gene. A suicide gene is used as a safety system, allowing cells expressing the gene to be killed by introduction of a selective agent. This is desirable in case the recombinant gene causes a mutation leading to uncontrolled cell growth, or the cells themselves are capable of such growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene. Typically, the suicide gene encodes for a protein that has no ill effect on the cell but, in the presence of a specific compound, will kill the cell. Thus, the suicide gene is typically part of a system.

In one embodiment, the suicide gene is active in NK-92 cells. In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir. In another embodiment, the suicide gene is cytosine deaminase, which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood. 1998 Jul. 15; 92(2):672-82. In a further embodiment, the suicide gene is cytochrome P450, which is toxic in the presence of ifosfamide or cyclophosphamide. See, e.g. Touati et al. “A suicide gene therapy combining the improvement of cyclophosphamide tumor cytotoxicity and the development of an anti-tumor immune response.” Curr Gene Ther. 2014; 14(3):236-46. In yet another embodiment, the suicide gene is iCasp9. Di Stasi, (2011) “Inducible apoptosis as a safety switch for adoptive cell therapy.” N Engl J Med 365: 1673-1683. See also Morgan, “Live and Let Die: A New Suicide Gene Therapy Moves to the Clinic” Molecular Therapy (2012); 20: 11-13. iCasp9 induces apoptosis in the presence of a small molecule, AP1903. AP1903 is biologically inert small molecule, that has been shown in clinical studies to be well tolerated, and has been used in the context of adoptive cell therapy.

Of course, it should be noted that all of the recombinant proteins can be expressed from individual recombinant sequences. However, it is generally preferred that where multiple recombinant sequences are expressed (e.g., CAR, CD16, cytokine), coding regions may be arranged in a polycistronic unit with at least two or at least three coding regions encoding the recombinant proteins. Therefore, transgenes can be engineered into an expression vector by any mechanism known to those of skill in the art. Where multiple transgenes are to be inserted into a cell, transgenes may be engineered into the same expression vector or a different expression vector. In some embodiments, the cells are transfected with mRNA encoding the transgenic protein to be expressed. In some embodiments, the cells are transfected with DNA encoding the transgenic protein to be expressed. Transgenes, mRNA and DNA can be introduced into the NK-92 cells using any transfection method known in the art, including, by way of non-limiting example, infection, viral vectors, electroporation, lipofection, nucleofection, or “gene-gun.”

In preferred embodiments, it should therefore be noted that the genetically modified NK cell (especially where the cell expresses a CAR and CD16 or variant thereof) will exhibit three distinct modes of cell killing: General cytotoxicity which is mediated by activating receptors (e.g., an NKG2D receptor), ADCC which is mediated by antibodies bound to a target cell, and CAR mediated cytotoxicity. As will be readily apparent, contemplated genetically modified cells can be used for treatment of various diseases, and especially of various cancers and viral infections where a diseased cell presents a disease-specific or disease-associated antigen. Consequently, the inventors contemplate methods of treating patients with modified NK or NK-92 cells as described herein. In one embodiment, the patient is suffering from cancer (e.g., a tumor) and the modified NK-92 cell or cell line expresses a CAR specific for an antigen expressed on the surface of a cell from the cancer or tumor. In one embodiment, the patient is suffering from a viral infection and the modified NK-92 cell or cell line expresses a CAR specific for an antigen expressed on the surface of a cell that has been infected by the virus. In one embodiment, the patient is suffering from a bacterial infection and the modified NK-92 cell or cell line expresses a CAR specific for an antigen expressed on the surface of a bacterial cell causing the infection.

In some embodiments, the cancer is selected from the group consisting of leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

Contemplated modified NK or NK-92 cells can be administered to an individual by absolute numbers of cells. For example, the individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) modified NK-92 cells per injection, or any ranges between any two of the numbers, end points inclusive. In other embodiments, modified NK-92 cells can be administered to an individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) modified NK-92 cells per kilogram of the individual, or any ranges between any two of the numbers, end points inclusive. In other embodiments, the total dose may calculated by m² of body surface area, including about 1×10¹¹, 1×10¹⁰, 1×10⁹, 1×10⁸, 1×10⁷, per m², or any ranges between any two of the numbers, end points inclusive. The average person is about 1.6 to about 1.8 m². In a preferred embodiment, between about 1 billion and about 3 billion NK-92 cells are administered to a patient.

The modified NK-92 cells, and optionally other anti-cancer or anti-viral agents can be administered once to a patient with cancer or infected with a virus or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In one embodiment, where the modified NK-92 cells express a suicide gene, the patient is administered an agent to trigger modified NK-92 cell death. In one embodiment, the agent is administered at a time point after administration of the modified NK-92 cells that is sufficient for the NK-92 cells to kill target cells.

In one embodiment, the modified NK-92 cells are irradiated prior to administration to the patient. Irradiation of NK-92 cells is described, for example, in U.S. Pat. No. 8,034,332, which is incorporated herein by reference in its entirety. In one embodiment, modified NK-92 cells that have not been engineered to express a suicide gene are irradiated.

Furthermore, it should be appreciated that contemplated treatments will also include administration of other immune therapeutic entities, and especially preferred immune therapeutic entities include a viral cancer vaccine (e.g., adenoviral vector encoding cancer specific antigens), a bacterial cancer vaccine (e.g., non-pyrogenic E.coli expressing one or more cancer specific antigens), a yeast cancer vaccine, N-803 (also known as ALT-803, ALTOR Biosciences), an antibody (e.g., binding to a tumor associated antigen or patient specific tumor neoantigen), a stem cell transplant (e.g., allogeneic or autologous), and a tumor targeted cytokine (e.g., NHS-IL12, IL-12 coupled to a tumor targeting antibody or fragment thereof).

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1: CAR mRNA Preparation

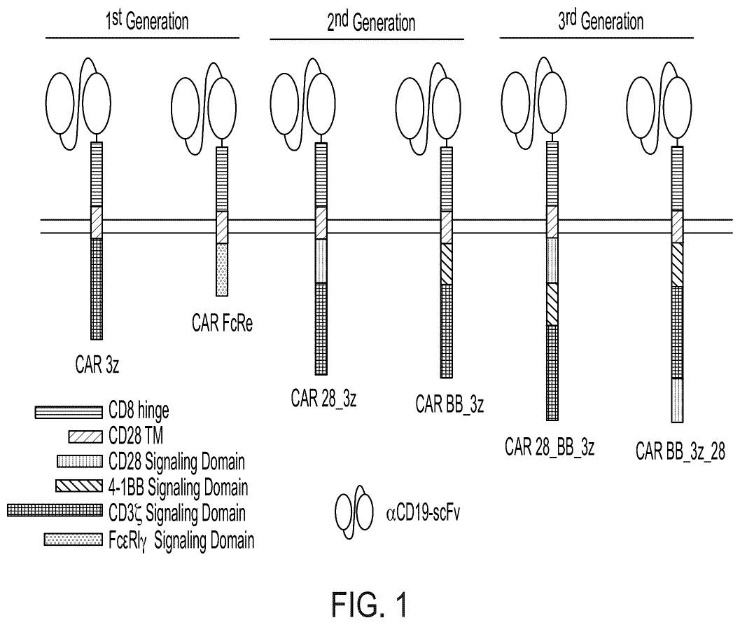

DNA sequences encoding each variant of CD19CAR schematically depicted in FIG. 1 were designed in silico, synthesized de novo, and subcloned into the mRNA expression vector, pXT7 (GeneArt, Life Technologies). Ten micrograms (μg) of plasmid were linearized by digestion with the Sall restriction enzyme (New England Biolabs) and purified using a QIAgen gel purification kit (QIAgen) according to manufacturer's instructions.

The linearized DNA was used as template for in vitro synthesis of mRNA using a T7 mMessage mMachine Ultra transcription kit (ThermoFisher Scientific, Waltham, Mass.) according to the manufacturer's instructions. This kit includes a polyadenylation extension step that increases the length of the polyA tail of the mRNA and thus enhances stability in vivo.

mRNA for six CD19-CAR variants was prepared, with a green fluorescent protein (GFP) mRNA prepared as a negative control. All of the CD19-CAR polypeptide variants contained an extracellular domain comprising an anti-CD19 scFv region (αCD19-scFv) (SEQ ID NO:4), a hinge region from CD8 (SEQ ID NO:6), and a transmembrane domain from CD28 (SEQ ID NO:7). The intracellular domains of the CD19CARs were as follows and schematically shown in FIG. 1: CAR 3z contained a CD3ζ signaling domain; CAR FcRe contained a FcϵRIγ signaling domain (SEQ ID NO: 1); CAR 28_3z contained a CD28 signaling domain fused to a CD3ζ signaling domain; CAR BB_3z contained a 4-1BB signaling domain fused to a CD3ζ signaling domain; CAR 28_BB_3z contained a CD28 signaling domain fused to a 4-1BB signaling domain fused to a CD3ζ signaling domain; CAR BB_3z_28 contained a 4-1BB signaling domain fused to a CD3ζ signaling domain fused to a CD28 signaling domain.

More particularly, the 1^{st} generation CAR with CD3ζ signaling domain of FIG. 1 had a nucleic acid sequence of SEQ ID NO:13 (human) and SEQ ID NO:21 (murine), which translated to an amino acid sequence of SEQ ID NO:22. The 1^{st} generation CAR with a FcϵRIγ signaling domain nucleic had a nucleic acid sequence of SEQ ID NO:5 and an amino acid sequence of SEQ ID NO:3. The 2^{nd} generation CAR with CD28/CD3ζ signaling domain had a nucleic acid sequence of SEQ ID NO:14 and the 2^{nd} generation CAR with 4-1BB/CD3ζ signaling domain had a nucleic acid sequence of SEQ ID NO:15. The 3^{rd} generation CAR with CD28/4-1BB/CD3ζ signaling domain had a nucleic acid sequence of SEQ ID NO:16 and the 3^{rd} generation CAR with 4-1BB/CD3ζ/CD28signaling domain had a nucleic acid sequence of SEQ ID NO:17.

Further 1^{st} generation CARs with a FcϵRIγ signaling domain were prepared as described in more detail below in which the hinge region was a CD8 hinge (SEQ ID NO:6 or SEQ ID NO:45 (human), encoded by SEQ ID NO:46), in which the transmembrane domain was a CD28 transmembrane domain (SEQ ID NO:7), and in which the signaling domain was a FcϵRIγ signaling domain (SEQ ID NO:1, encoded by nucleic acid SEQ ID NO:2).

Target specificity was then imparted against a variety of tumor-associated targets using selected scFv portions as follows (all in a sequential arrangement as shown in FIG. 1 CAR FcRe): CD19 (using anti-CD19 scFv of SEQ ID NO:4 or SEQ ID NO:24, encoded by codon-optimized SEQ ID NO:23), CD20 (using anti-CD20 scFv of SEQ ID NO:26, encoded by codon-optimized SEQ ID NO:25), CD33 (using anti-CD33 scFv of SEQ ID NO:28, encoded by codon-optimized SEQ ID NO:27), CSPG4 (using anti-CSPG4 scFv of SEQ ID NO:30, encoded by codon-optimized SEQ ID NO:29), EGFR (using anti-EGFR scFv of SEQ ID NO:32, encoded by codon-optimized SEQ ID NO:31), IGF1R (using anti-IGF1R scFv of SEQ ID NO:34, encoded by codon-optimized SEQ ID NO:33), CD30 (using anti-CD3OscFv of SEQ ID NO:36, encoded by codon-optimized SEQ ID NO:35), HER2/neu (using anti-HER2/neu scFv of SEQ ID NO:38, encoded by codon-optimized SEQ ID NO:37), GD2 (using anti-GD2 scFv or SEQ ID NO:40 or SEQ ID NO:42, encoded by codon-optimized SEQ ID NO:39 or SEQ ID NO:41), CD123 (using anti-CD123 scFv of SEQ ID NO:49, encoded by codon-optimized SEQ ID NO:48), PD-L1 (using anti-PD-L1 scFv of SEQ ID NO:51, encoded by codon-optimized SEQ ID NO:50), B7-H4 (using anti-B7-H4 scFv of SEQ ID NO:53, encoded by codon-optimized SEQ ID NO:52), and FAP (using anti-FAP scFv of SEQ ID NO:58 or SEQ ID NO:59, encoded by codon-optimized SEQ ID NO:56 or SEQ ID NO:57).

Likewise, target specificity was imparted against a variety of virus-associated targets using selected scFv portions as follows (all in a sequential arrangement as shown in FIG. 1 CAR FcRe): HIV gp120 (using anti-gp120 scFv of SEQ ID NO:55, encoded by codon-optimized SEQ ID NO:54).

All constructs as prepared above expressed well in NK-92 cells and exemplary results are shown for the physiological activity of the so modified NK-92 cells.

### Example 2: Electroporation of NK-92 Cells with CD19CAR mRNA

NK-92 cells were grown in X-Vivo10 medium (Lonza, Basel, Switzerland) supplemented with 5% Human AB Serum (Valley Biomedical, Winchester, Va.) and 500 IU/mL IL-2 (Prospec, Rehovot, Israel). Cells were electroporated with mRNA using the Neon™ electroporation device (Life Technologies, Carlsbad, Calif.), following the manufacturer's parameters for NK-92 cells (1250 V, 10 ms, 3 pulses) and using 5 μg of mRNA per 10⁶ cells in a volume of 100 μl. Electroporated cells were maintained in medium (same as above) for 20 hours (h).

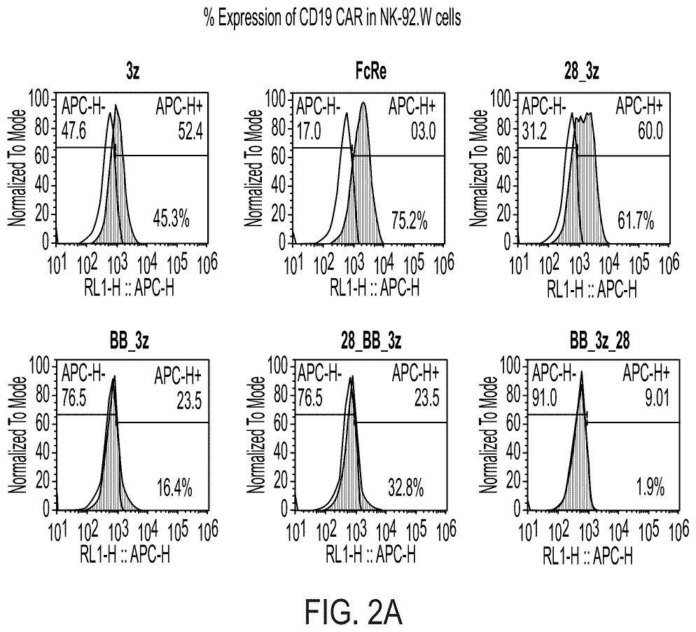

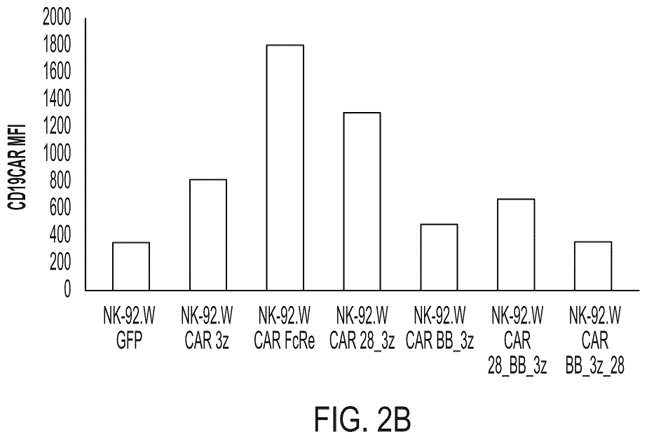

The CD19CAR expression on the NK-92 cell surface was determined by flow cytometry using anti-scFv antibody labeled with eF660 (eBioscience, San Diego, Calif.). FIG. 2A shows the % expression of the indicated CD19CAR in the NK-92 cell population. FIG. 2B shows the median fluorescence intensity (MFI, minus background) of cells electroporated with the indicated CD19CAR. As can be taken from FIGS. 2A and 2B, CAR FcRe unexpectedly had the highest percentage of cells (75.2%) expressing CD19CAR at the cell surface, as well as the highest MFI (quantity of expressed CAR on a recombinant cell), followed by 28_3z (61.7%).

### Example 3: Cytotoxicity of NK-92 Cells Expressing CD19CAR Against Cancer Cell Lines

The efficacy of CAR-expressing NK-92 cells to target cancer cells in vitro was tested 20 hours post-electroporation using a flow-based in vitro cytotoxicity assay. Effector cells (NK-92 expressing CD19CAR or GFP) were mixed with PKHGL67-labeled (Sigma-Aldrich, St. Louis, Mo.) target cells (K562; or SUPB15, B-ALL, CD19^{k}) at different effector to target ratios (5:1 to 0.3:1) in a 96-well plate and incubated 4 hat 37° C. Propidium Iodide (PI) (Sigma Aldrich, St. Louis, Mo.) was added to the cells and samples were analyzed within 2 h using an Attune flow cytometer (Life Technologies, Carlsbad, Calif.). The cytotoxicity was determined by the % of PI-positive cells within the PKH-positive target population.

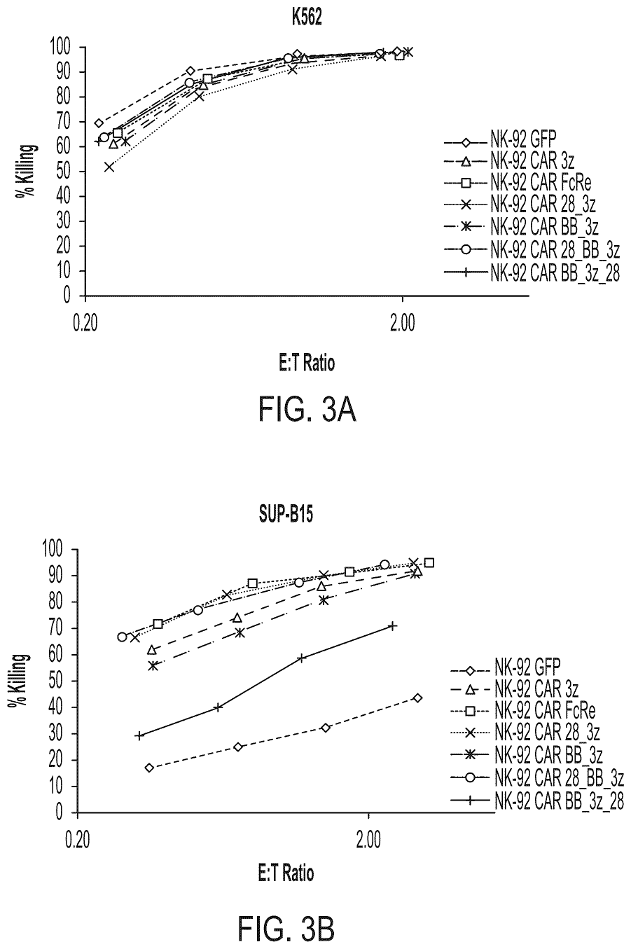

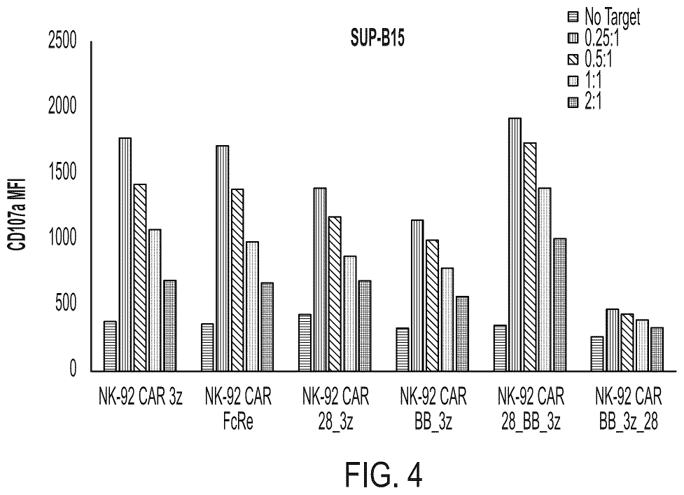

Exemplary results are provided in FIGS. 3A and 3B. NK-92 cells are effective at killing K562 cells regardless of CD19CAR expression as can be seen from FIG. 3A. Thus, it should be noted that recombinant cells will not lose cytotoxicity. In contrast, GFP-expressing NK-92 cells were inefficient at killing the cancer cell line SUP-B15. SUP-B15 is an acute lymphoblastic leukemia cell line that is CD19-positive and resistant to NK-92-mediated cytotoxicity. Expression of any CD19CAR tested provided increased cytotoxic activity against the SUP-B15 cell line compared to control (GFP-expressing NK-92 cells) as can be readily taken from FIG. 3B. Surprisingly, CAR FcRe exhibited cytotoxicity similar or superior to the 2^{nd} and 3^{rd} CARs. Such finding is particularly unexpected as the FcϵRIγ signaling domain was present only as a single unit and not combined with other signaling domains. Such arrangement, when used in CAR T-cells failed to provide desirable targeted cytotoxicity. Advantageously, tricistronic mRNA constructs were able to produce substantial quantities of desired CARs with excellent functional activity. Such constructs are especially beneficial where the CAR expression should be transient.

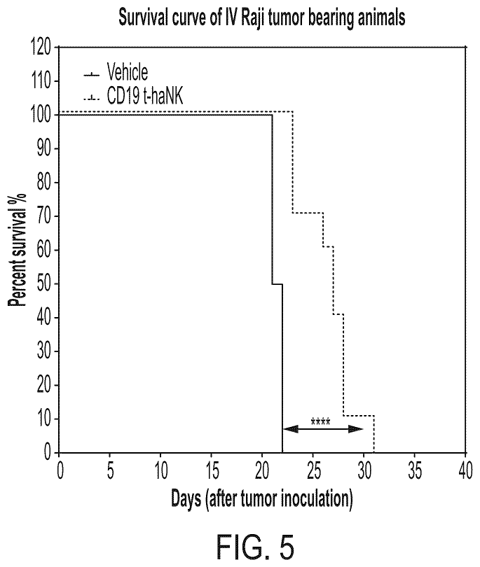

Degranulation is a critical step required for the release of the lytic proteins (e.g., perforin and granzyme) from secretory granules in the NK-92 cells. Degranulation is initiated by recognition of a target cell by NK-92. To test degranulation in the constructs, effector cells (NK-92) were mixed with unlabeled target cells (SUP-B15) at different effector to target ratios (5:1 to 0.3:1) in a 96-well plate, and anti-CD107a (FITC-conjugated, BD Pharmingen, San Jose, CA) was added to each well. Plates were incubated at 37° C. in a CO₂ incubator and after 1 h monensin (Golgi-stop) was added to the wells. The plates were incubated for another 3 h at 37° C. and the samples were analyzed by flow cytometry (Attune, Life technologies, Carlsbad, Calif.). Percentage degranulation was determined by subtracting the % CD107a positive in NK-92 cells alone to the % CD107a positive in the effector+target samples, and exemplary results are provided in FIG. 4.

### Example 4: CD19 t-haNK Cells Significantly Improved Animal Survival in a Raji Tumor Xenograft Model

CD19 t-haNK cells (clone 19.6) comprising the Fc Epsilon intracellular signaling domain. CD19 t-haNK cells were cultured in X-VIVOTM 10 medium supplemented with 5% heat inactivated human AB serum.

Test Animals: Animal Strain/Species: NOD.Cg-PrkdcscidIl2rgtm1Wjl/SzJ (NSG) mice; Age: 9-10 weeks at study initiation (after quarantine); Sex: Female; Body Weight: 20-27 grams at study initiation. Number of Animals: 20 for the IV tumor model; 12 for the SC tumor model. Supplier: The Jackson Laboratory (610 Main Street Bar Harbor, Me. 04609 US).

Raji Tumor Model: Raji Cancer Cell Line: Raji cells were originally purchased from ATCC (Catalog #CCL-86TM; Lot #61723871) and then expanded and prepared for administration.

Cell Culture Medium: ATCC-formulated RPMI-1640 medium supplemented with 10% fetal bovine serum with penicillin (100 U/mL), streptomycin (100 μg/mL).

Cell Harvest: Raji cells (passage 12) in exponential phase were collected by centrifugation. Cells were washed and re-suspended in serum free medium at the concentration of 5×10⁵ viable cells/mL for IV inoculations, and in medium/Matrigel (1:1 v/v) at the concentration of 2.5×10⁶ viable cells/mL for SC implantations. Cells were stored on ice prior to animal injection. Cells used in the in vivo study had a viability of 96%.

Raji Cell Inoculation: Raji IV Model. 20 animals were injected IV via the lateral tail vein with 0.2 mL of Raji cell suspension with 27 gauge needles (1×10⁵ cells inocula). Raji SC Model. 12 animals were implanted SC on both flanks with 0.1 mL of Raji cell suspension with 25-gauge needles (2.5×10⁵ cells inocula).

Other Reagents: RPMI-1640 media, X-VIVOTM 10 media; Heated-inactivated human male AB serum (Access Cell Culture (Access Biologicals LLC); Fetal Bovine Serum (FBS); Pen Strep Glutamine (100 X) (Life Technologies, Catalog #10378, Lot #1881463, Expiration date: May 2018); Matrigel Basement Membrane Matrix; Pluronic(R) F-68, 10% Solution.

### EXPERIMENTAL PROCEDURES

IV Raji Model—Randomization: Within 24 hours after cancer cell inoculation, which was defined as Day 1, 20 animals were pseudo-randomized into 2 groups of 10 according to body weight to achieve similar average body weight between the groups.

Test Article Administration: On Days 2, 5, 8, 10, 12, and 17, CD19 t-haNK cells grown in the exponential phase were harvested by centrifugation and formulated in X-VIVO™ 10 at the concentration of 5×10⁷ cells/mL for IV administration at the dose of 1×10⁷ cells per mouse with an injection volume of 200 μL. Animals in Group A received the vehicle control, while animals in Group C received CD19 t-haNK cells.

Body Weight: Animals were weighed prior to tumor cell injection and twice weekly.

Clinical Observations: Animals were observed daily for mortality/morbidity (GO to G4) and clinical signs of toxicity. Paralyzed or moribund animals were euthanized.

Euthanasia: Animals were euthanized with CO₂ inhalation followed by cervical dislocation. Mortality events (euthanasia or spontaneous) were recorded in Death Log (Appendix 6) and tallied to calculate the survival curve.

SC Raji Model—Tumor Volume Measurement: After SC tumor implantation, animals were examined at least twice a week for tumor establishment. When tumors became palpable, tumor volumes (TV) were measured with a digital hand held caliper once to twice weekly, and calculated using this formula: TV=Length×Width 2/2 [Length being the greatest diameter and Width being the shortest diameter of the tumor].

Randomization: When the average tumor volume reached an injectable size (195 mm3 in this case; 24 days post-implantation), the 12 tumor-bearing animals were pseudo- randomized into 2 groups of 6 to achieve similar tumor volumes between the groups. This was defined as Day 0.

Test Article Administration: On Days 1, 4, 7, 9, 11, and 13, CD19 t-haNK cells grown in the exponential phase were harvested by centrifugation, subjected to 1000 cGy gamma irradiation, and formulated in X-VIVO 10 medium at the concentration of 5×10⁷ cells/mL for IV administration at the dose of 1×10⁷ cells per mouse with an injection volume of 200 μL. As shown in Table 1, animals in Group D received the vehicle solution, while animals in Group F received CD19 t-haNK cells.

Body Weight. Animals were weighed prior to tumor cell injection and then twice weekly.

Clinical Observations. Animals were observed daily for mortality/morbidity (GO to G4) and clinical signs of toxicity (T1 to T12). Paralyzed or moribund animals were euthanized.

Endpoint and Euthanasia. While moribund animals were euthanized as soon as they showed morbidity, surviving animals were subjected to scheduled euthanasia for tissue collection. Specifically, half of the surviving animals (up to 3 mice/group) were euthanized on Day 13 at 6 hours post the last dose of test article administration. The rest of the animals were euthanized on Day 15 at 48 hours post the last dosing.

Necropsy and Tumor and Tissue Collection. Upon termination, a necropsy was performed and organs with visible gross lesions were collected, fixed in 10% formalin, and submitted to a contract pathology laboratory (Seventh Wave Laboratories) for histological evaluation of tumor/metastatic disease burden.

TABLE 1

| | | Tumor | | Tx | NK Cell | Treatment | |
|---|---|---|---|---|---|---|---|
| Group | N | Model | Treatment | Route | Dose | Days | Endpoint |
| A | 10 | IV | Vehicle | IV | / | 2, 5, 8, 10, | Moribund |
| | | | | | | 12, and 17 | |
| C | 10 | IV | CD19 | IV | 1 × 10⁷ | 2, 5, 8, 10, | Moribund |
| | | | t-haNK, | | | 12, and 17 | |
| | | | non-IR | | | | |
| D | 6 | SC, | Vehicle | IV | / | 1, 4, 7, 9, | Days 13 and 15 |
| | | bilateral | | | | 11, and 13 | or Moribund |
| F | 6 | SC, | CD19 | IV | 1 × 10⁷ | 1, 4, 7, 9, | Days 13 and 15 |
| | | bilateral | t-haNK, IR | | | 11, and 13 | or Moribund |

IR, irradiated (1000 cGy); non-IR, non-irradiated; IV, intravenous; SC, subcutaneous; Tx, treatment.

Data Analysis

Tumor Volume Calculation: Tumor volume=Length×Width 2/2 (Length and Width being the longest and shortest diameters of the tumor, respectively); Tumor Growth Inhibition (TGI) Calculation: TGI=(TC−Tt)/ΔTC×100%, where TC and Tt is the average tumor volume for control and treatment groups at the end of the study, respectively, and ΔTC is the change in average tumor volume in the control group.

Statistical Analysis—Tumor Growth Curves: Tumor growth curves were analyzed by 2-way ANOVA followed by multiple comparison by Tukey test. Survival Curves: Survival curves were analyzed by Log-rank (Mantel-Cox) test.

Liver Metastasis Estimation: Differences in liver metastatic disease burden on individual days were analyzed by unpaired 2-tailed t test. Statistical Significance: P<0.05 is considered statistically significant. All statistical analyses were performed using GraphPad Prism version 7.

Results

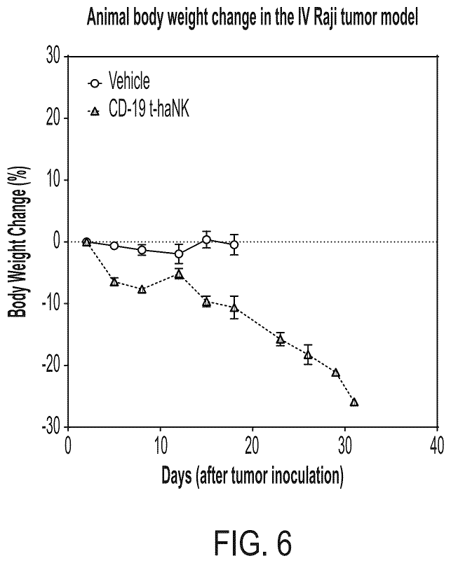

IV Raji Model: The main readout in the IV tumor model was animal survival. A death event was counted when an animal was found dead or was euthanized due to disease- related morbidity and/or paralysis. As shown in FIG. 5, compared to vehicle control, CD19 t-haNK cell treatment was able to significantly improve the animals' rate of survival, resulting in a median survival of 27 days versus 21.5 days in the vehicle control group (P<0.0001).

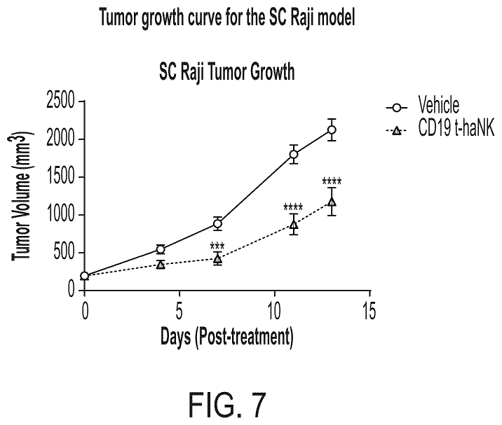

Animal body weight change was also monitored throughout the study. As shown in FIG. 6, CD19 t-haNK treated animals demonstrated a moderate (less than 10%) and short-term body weight loss when treatment was first initiated, which is not an uncommon phenomenon in animals receiving IV NK infusions, and not specific to the CD19 t-haNK cells (Reference study: LABC-TX01701). Their body weight was able to recover after the first week of treatment before decreasing again due to disease progression.

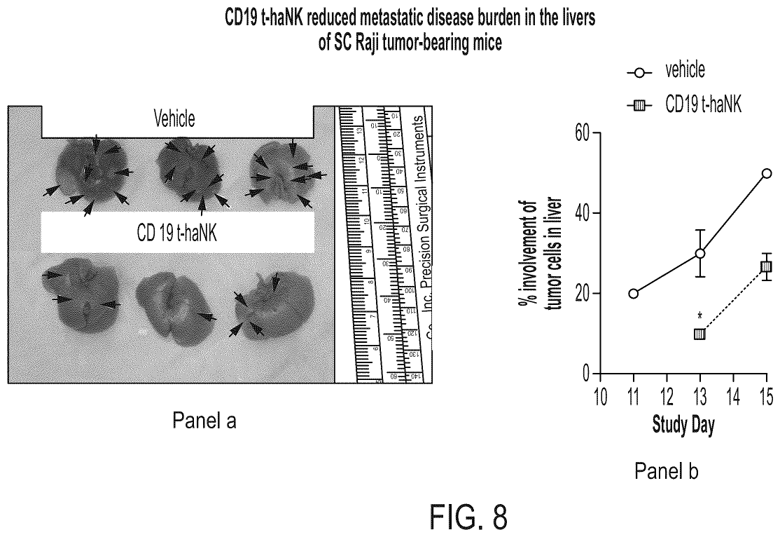

SC Raji Model: The primary readout in the SC tumor model was tumor growth. As shown in FIG. 7, CD19 t haNK cells demonstrated evident and statistically significant tumor growth inhibition on and after Day 7 compared to the vehicle control group, with a 49% TGI at the end of the study (Day 13).

Further, as Raji is an aggressive lymphoma model, even when inoculated SC, the cancer cells were able to disseminate and develop multiple sites of metastases that eventually led to animal morbidity and/or death. There were a total of 3 animals (50%) that were moribund between Days 11 and 13 and therefore were euthanized in the vehicle group. In contrast, there was no unscheduled death event in the CD19 t-haNK cells group (Table 3).

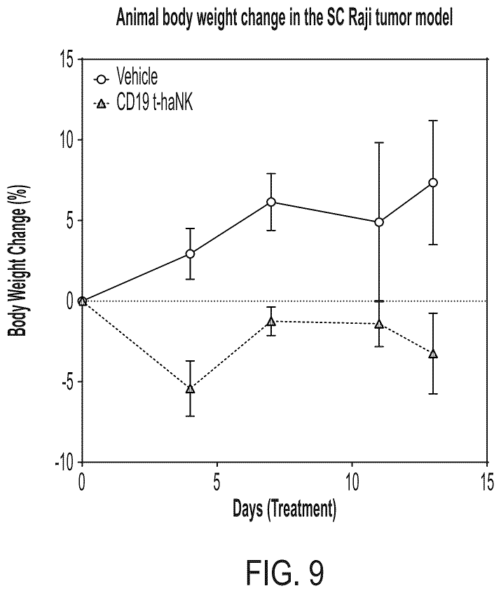

In addition, a qualitative reduction of liver metastases was observed in CD19 t-haNK treated animals during necropsy (FIG. 8A). A semi-quantitative estimation of the disease burden was performed by a contract pathology lab (Seventh Wave Laboratories) on H&E stained liver sections that were representatively sampled. As summarized in FIG. 8B and Table 4, there was a clear trend of increasing disease burden as the study advanced. Livers of CD19 t-haNK treated animals exhibited a remarkably lower percentage of cancer infiltrated areas compared to the vehicle control. Due to the small sample number and unscheduled early mortality in the control group, statistical analysis could only be performed on the Day 13 data. This analysis showed a significant difference in disease burden, with an average of 10% infiltration in CD19 t-haNK treated animals versus 30% in the control group.

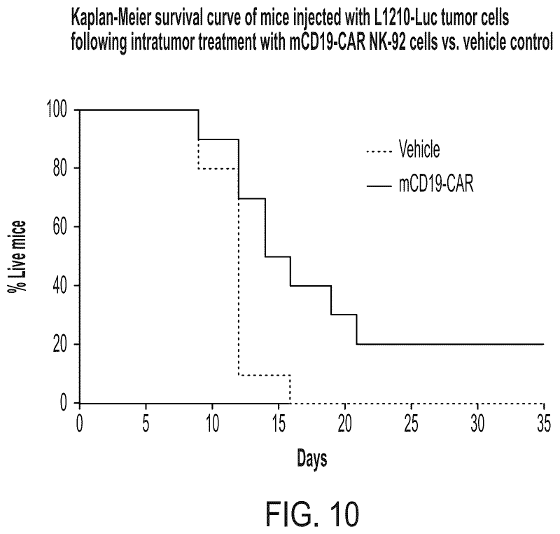

Body weight change was monitored throughout the study, and similar to the IV Raji model, CD19 t-haNK treated animals demonstrated a moderate (less than 10%) and transient body weight loss in the beginning of the treatment regimen as can be taken from FIG. 9.

TABLE 2

| Group | Initial N | Day 11 | Day 13 | Day 15 |
|---|---|---|---|---|
| D | 6 | 2 × Moribund | 1 × Moribund | |
| (Vehicle) | | | 2 × Scheduled | 1 × Scheduled |
| F (CD19 | 6 | | | |
| t-haNK) | | | 3 × Scheduled | 3 × Scheduled |

Scheduled: scheduled euthanasia for tissue collection.

TABLE 3

| Group | Animal | Collection | Estimated % | |
|---|---|---|---|---|
| (Treatment) | Number | Day | Tumor | Mean |
| D | 503 | 11 | 20 | 20 |
| (Vehicle) | 520 | 11 | 20 | |
| | 487 | 13 | 40 | 30 |
| | 488 | 13 | 20 | |
| | 497 | 13 | 30 | |
| | 502 | 15 | 50 | 50 |
| F (CD19 | 495 | 13 | 10 | 10 |
| t-haNK) | 505 | 13 | 10 | |
| | 507 | 13 | 10 | |
| | 512 | 15 | 30 | 27 |
| | 522 | 15 | 30 | |
| | 525 | 15 | 20 | |

To assess the anti-tumor efficacy of CD19 t-haNK cells in repeated IV dosing regimens, 2 variations of the Raji xenograft model with IV and SC tumor inoculations, respectively, were utilized in this study.

In the IV tumor model, CD19 t-haNK cells were able to significantly improve animal survival, prolonging median survival by 5.5 days (a 26% increase) compared to the vehicle control group. In the SC tumor model, CD19 t-haNK cells were able to significantly suppress tumor growth, resulting in a 49% TGI at the end of the study. Furthermore, CD19 t-haNK treatment was able to reduce the number of animal morbidity/death events ( 0/6 in CD19 t-haNK treated animals versus 3/6 in the control group), and markedly decrease metastatic disease burden in the liver of SC Raji-tumor bearing animals.

As can be seen from the above data, CD19 t-haNK cells displayed significant therapeutic efficacy compared to vehicle control in both variations of the Raji xenograft model.

### Example 5. Treatment of Mice having L1210 Tumors with CD19-CAR-NK-92 Cells Increased Survival, and Mice that Completely Responded to Treatment Rejected L1210 Tumor Allografts when Re-Challenged

Experimental Design: Thirty (30) male DBA/2J mice aged 6-8 weeks (Jackson Laboratories) were enrolled following randomization on Day 0. All animals were housed under standard environmental conditions and maintained on LabDiet 5053 irradiated rodent chow and sterile water provided ad libitum. On arrival, animals were identified by ear punch and housed in cages of ten (10) and acclimated in place for a minimum of three days prior to commencement of the study. Following acclimation, the injection area of each mouse was shaved and cleaned with sterile EtOH swab. On Day PRO (pre-randomization Day 0), animals were anesthetized with isoflurane for tumor cell injection. All animals were injected with 2×10⁵ L1210-Luc tumor cells subcutaneously (s.c.) into the right flank in a volume of 0.1 mL serum-free DMEM on Day PRO. Beginning on Day PR 7, all animals had tumors measured daily by digital caliper. On ˜Day PR7 when tumor volumes were measured at ˜50-150 mm³, and mean tumor volume was measured at ˜100 mm³, the twenty (20) animals bearing tumors nearest to ˜100mm′ were selected for enrollment in the study; these animals were randomized into two (2) groups consisting of ten (10) animals each. Randomization day was considered Day 0 of the study, and administration of treatments commenced on this day. Animals not enrolled on study were immediately euthanized by CO2 overdose. Animals in Group 1 were administered vehicle (serum free DMEM) as an intratumoral (i.t.) injection of 50 μl. Animals in Group 2 were administered 2×10⁶ mCD19-CAR-aNK cells i.t. in a volume of 50 μl. Identical treatments were administered on Days 0, 2 and 4 of the study.

Animals were weighed and monitored for general health daily. Following randomization, tumors were measured by digital caliper three times each week (3×/week). Any animal bearing a tumor >2500 mm³ or a tumor that has ulcerated; that lost >30% of its initial body weight (on Day 0); or was found moribund, distressed or paralyzed was euthanized by CO₂ overdose with cause of death/sacrifice noted. On Day 30, completely responding animals and five (5) naive additional male DBA/2J mice aged ˜10 weeks (Jackson Laboratories; Barrier) comprising Group 4 were administered a rechallenge tumor cell inoculation of 2×105 L1210-Luc tumor cells subcutaneously (s.c.) into the left flank in a volume of 0.1 mL serum-free DMEM. All animals continued to be weighed and monitored daily and tumor measurements continued 3×/week through Day 60.

Results

Animal Survival to Welfare Thresholds—Initial Tumor Challenge: Animals were monitored for survival daily. Animals requiring euthanasia according to animal health and welfare thresholds, including loss of greater than 30% of their initial body weight, tumors exceeding 2500 mm³, inability to obtain food/water, or found moribund, were included for survival analysis. Animals requiring euthanasia due to ulcerated tumors were not included in survival analysis.

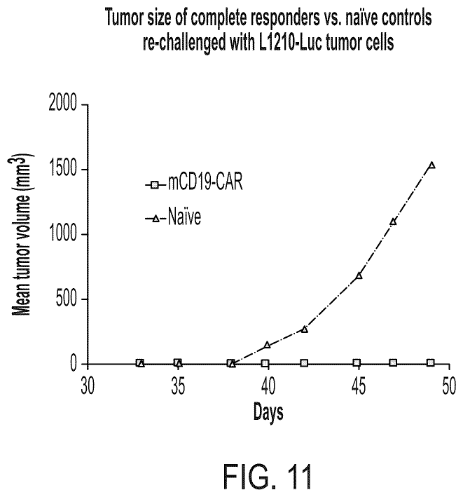

Cumulative survival to animal welfare thresholds over time is shown in FIG. 10. L1210 is an extremely fast-growing, aggressive tumor cell line and 0% of vehicle treated control animals survived further than twenty-three (23) days post tumor challenge. In contrast, treatment with CD19-CAR-aNK cells enhanced survival compared to treatment with vehicle. Indeed, 25% ( 2/8) of animals treated with CD19-CAR-aNK cells survived through study completion at Day 61 through tumor graft challenge.

The statistical significance of the observed survival enhancements provided by the test treatments was assessed by Log-rank (Mantel-Cox) and Gehan-Breslow Wilcoxon tests. Treatment with mCD19-CAR-aNK cells produced a statistically significant enhancement of survival, (p=0.05 (Mantel-Cox); p=0.04 (Gehan-Breslow-Wilcoxon). These results indicate that treatment with CD19-CAR-aNK produced statistically significant improvement of survival to welfare threshold compared to vehicle in this preclinical subcutaneous model of murine lymphocytic leukemia.

Tumor Re-challenge of Complete Responders: On Day 33, the two (2) complete responding animals from Group 2, along with five (5) age-matched naive animals were challenged/rechallenged with a second inoculum of 2×10⁵ L1210-Luc cells, injected into the opposite (left) flank (primary tumor was seeded into the right flank). Animals were monitored for survival daily. Animals requiring euthanasia according to animal health and welfare thresholds, including loss of greater than 30% of their initial body weight, tumors exceeding 2500 mm³, inability to obtain food/water, or found moribund, were included for survival analysis. Animals requiring euthanasia due to ulcerated tumors were not included in survival analysis.

All (5 of 5) survival analysis eligible naive animals required euthanization due to tumor volume by Day 52; in contrast, all completely responding animals previously treated with 2M CD19-CAR-aNK (N=2) cells survived through study completion (Day 62). The statistical significance of the observed survival enhancement provided by the test treatments was assessed by Log-rank (Mantel-Cox) and Gehan-Breslow Wilcoxon tests, however the enhancement in survival was not statistically distinguishable, most likely to due to small sample sizes.

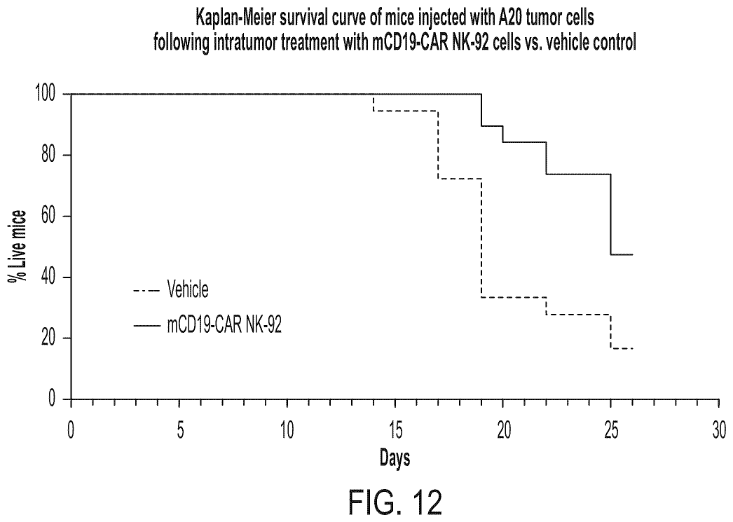

Tumors continued to be measured three times each week (3 ×/week) during the rechallenge phase. The mean tumor volume+SEM for each group from administration of challenge/rechallenge L1210-Luc cells to 0% control group survival (Day 52) are displayed in FIG. 11.

Tumors of naive animals were first detectable about seven days after administration (on study Day 40) and increased steadily and rapidly. In contrast, no tumors were detected following rechallenge into completely responding animals previously treated with 2M CD19-CAR-aNK cells at any point over the full course of the rechallenge phase (Day 33-61).

The data provided in this example suggest that completely responding animals previously treated with 2M CD19-CAR-aNK cells may have developed an effective immune response to L1210 tumor cells.

### Example 6: Treatment of Mice having A20 Tumors with mCD19-CAR-NK-92 Cells Increased Survival, and Mice that Completely Responded to Treatment Rejected A20 Tumor Allografts when Re-Challenged

### EXPERIMENTAL DESIGN

Part A: Forty (40) 5-7 week old BALB/c mice (20 males and 20 females) were sourced Taconic Biosciences to serve Part A. On pre-randomization (PR) Day 0, animals were injected subcutaneously (s.c.) into the left flank with 2.5×10⁶ A20 murine lymphoma cells in 100 μL volume of serum free media. Beginning on Day PR7, tumors were measured daily. Ten (10) days after tumor cell implantation (Day PR10; Day 0), mice were randomized into treatment groups, such that each group contained animals bearing tumors of similar volume and range. The day of randomization was considered Day 0 of the study. Tumors were measured three times each week (3×/week) by digital caliper to monitor tumor growth until completion of Part A on Day 26.

On Day 0, Day 3, and Day 5, mice were injected intratumorally (i.t.) with test cells or vehicle in 50 μl volume of serum free media into the tumor mass of each animal according to pre-established i.t. procedure (see Experimental Procedures). Briefly, animals were administered vehicle only or were administered 5×10⁶ mCD19-CAR-NK-92. On Day 26, animals that did not develop a tumor of volume >40 mm³ were unenrolled from the study and euthanized by CO₂ asphyxiation; enrolled animals that displayed a complete response to treatment (CR; tumors >40 mm³ regressing so as to be undetectable (0 mm³) over multiple days without relapse prior to Day 26) were enrolled in Part B.

Part B: Part B began on Day 26. Animals from Part A without tumors were enrolled in Part B, along with twelve (12) naïve animals (6 males and 6 females). All Part B animals were administered 2.5×10⁶ A20 cells into the right flank. Tumors were measured 2 times/week. Animals were euthanized on Day 57.

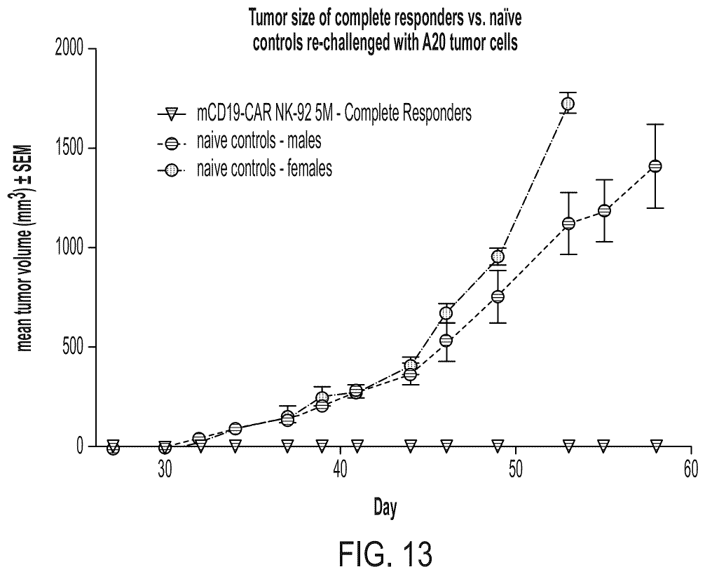

Results: Part A—Animal Survival: Animals were monitored for general health and survival daily. Animals requiring euthanasia according to animal health and welfare thresholds, including loss of greater than 30% of their initial body weight, tumors exceeding 1500 mm³, inability to obtain food/water or found moribund were included for survival analysis. Animals requiring euthanasia due to ulcerated tumors were not included in survival analysis. In this study, all animals considered in survival analysis were euthanized due to tumor burden exceeding 1500 mm³. As a subcutaneous tumor burden threshold represents an arbitrary cut-off point, the analysis of “survival” in this case must be considered only as an indicator of relative tumor growth. Cumulative survival over time for all animals considered is displayed in FIG. 12.

Of control animals administered vehicle intratumorally (i.t.) on Days 1, 3, and 5: 0 of 15 animals (0%) survived to Part A completion on Day 26. Survival through Day 26 was increased for animals for all animals receiving treatment: 9 out 18 (50%) animals administered 5M mCD19-CAR-NK92 cells. All groups were intercompared by log-rank (Mantel-Cox) test. Compared to animals administered vehicle, a statistically significant enhancement of survival was observed for animals administered 5M mCD19-CAR-NK92 cells (p=<0.0001). These results suggest that all treatments improved survival through Day 26 compared to treatment with vehicle.

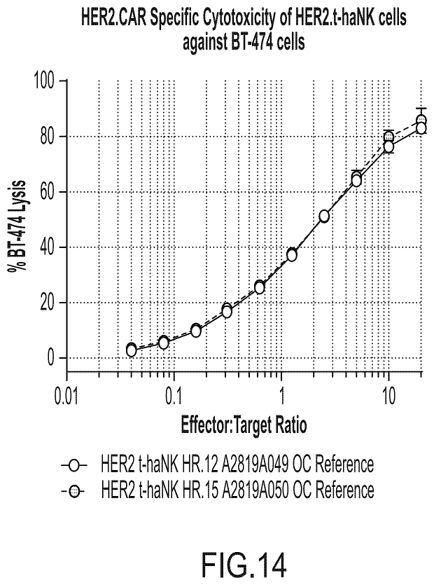

Part B—Tumor Re-challenge of Complete Responders: Animals that completely responded to treatment (bearing a tumor >40 mm³ that responded to treatment over the course of Day 0-26 (Part A) such that the tumor volume measured 0.00 mm³ through Day 26 without regrowth or relapse) were re-challenged with a second subcutaneous inoculation into the flank (opposite side from the first graft), with 2.5×10⁶ A20 tumor cells in 0.1 mL serum free RPMI-1640 media on Day 27; the rechallenge portion of the study was designated as Part B. An additional twelve animals were enrolled into Part B the study to serve as naïve controls; six (6) male and six (6) female age-matched BALB/c mice sourced at the same time and vendor as Part A mice were administered 2.5×10⁶ A20 tumor cells on Day 27. Tumors were measured 3 times/week for all animals through Day 57. Mean tumor volumes+SEM of each Part A treatment group and naïve controls are shown in FIG. 13. Tumors derived from cell inoculations into naïve animals grew steadily as expected; whereas re-challenge tumor cell inoculations into complete responder animals did not produce viable tumors (>40 mm³).

In summary, the data presented in this example indicates that, in contrast to naive mice, previously treated mice that completely responded to treatment were able to reject A20 tumor allografts applied as re-challenge regardless of the treatment, and suggests that that these animals developed a memory response to tumor antigens.

The following examples for targeted CAR constructs and associated functional data were from linearized DNA vector constructs, which allowed transfected cells to integrate the linearized DNA into the genome and to so provide an avenue for non-transient expression of the specific CARs.

### Example 7: HER2-CAR with FcϵRIγ Signaling Domain

In this example, the inventors constructed a 1^{st} generation CARs with a FcϵRIγ signaling domain that included an anti-HER2 scFv coupled to a CD8 hinge, that in turn was coupled to a CD28 transmembrane domain, which was coupled to a FcϵRIγ signaling domain. The so constructed HER2-CAR had a nucleic acid sequence of SEQ ID NO:60.

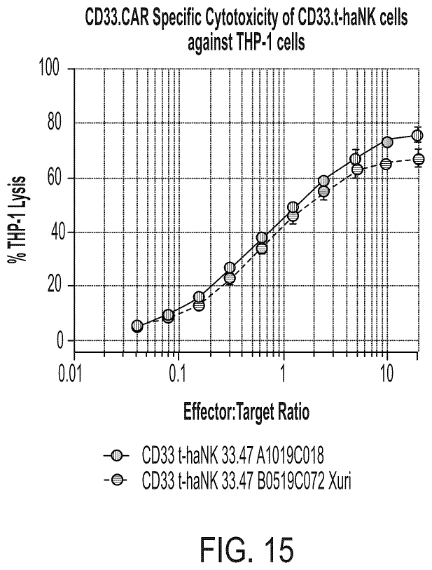

Functionality of the so constructed HER2.CAR-t-haNK cells was tested against BT-474 cells using a standard cytotoxicity assay and exemplary results are shown in FIG. 14. As can be readily seen from the data, the HER2.CAR-t-haNK cells expressing the CAR with the FcϵRIγ signaling domain exhibited significant cytotoxicity against the BT-474 target cells.

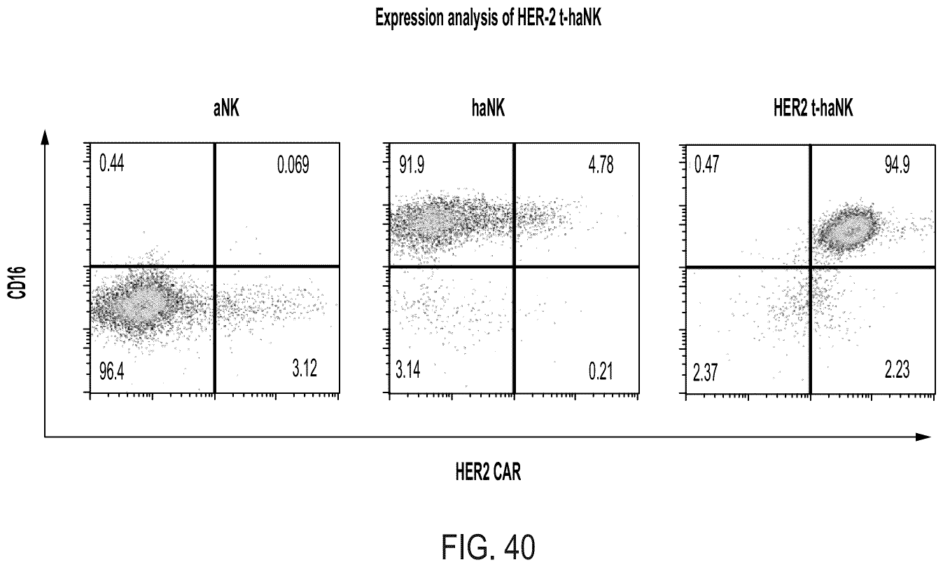

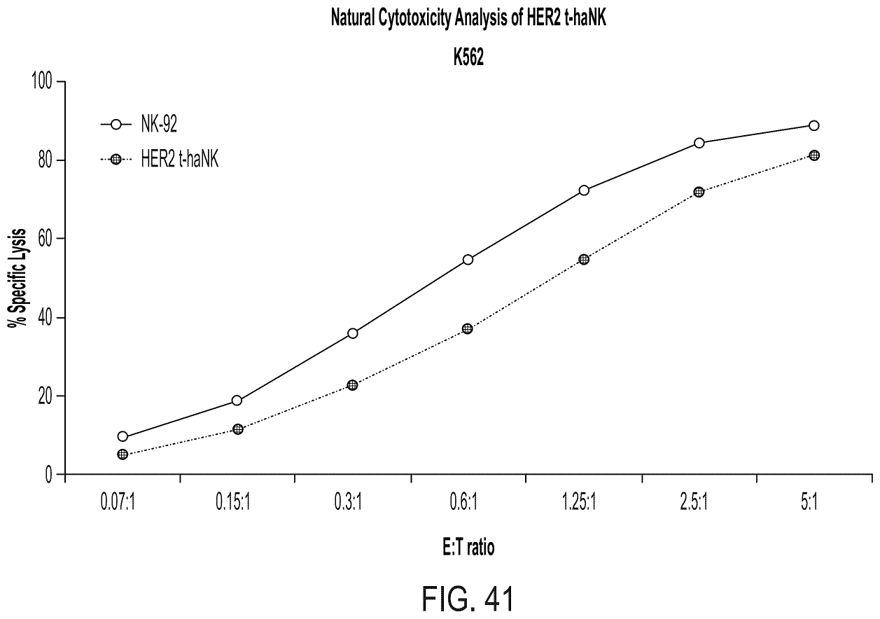

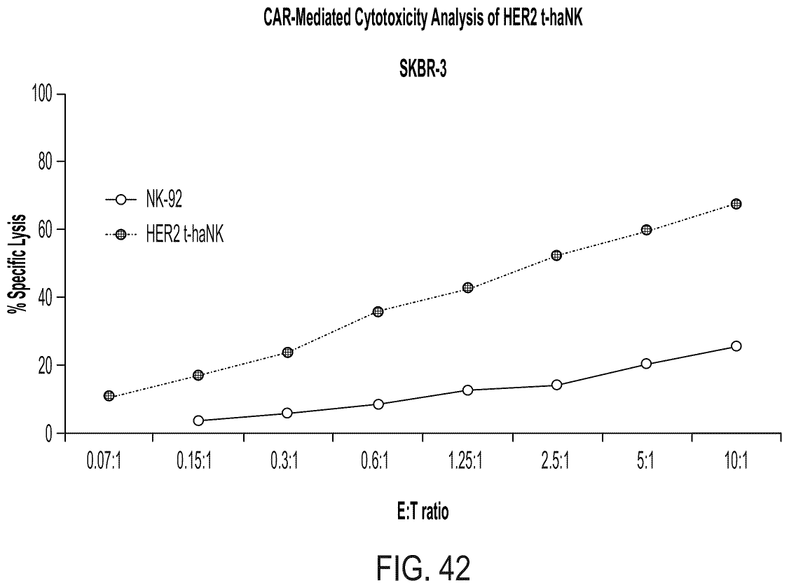

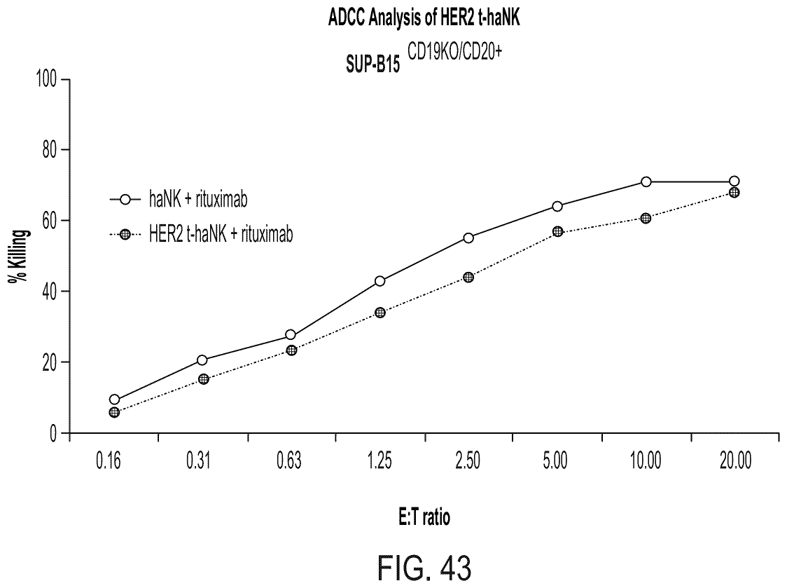

In further experiments, the inventor demonstrated expression of the HER2.CAR in HER2.CAR-t-haNK cells as is illustrated in FIG. 40. Natural cytotoxicity of the HER2.CAR-t-haNK cells is shown in the results of Fig.41, while results for CAR mediated cytotoxicity are shown in FIG. 42. Exemplary data for ADCC of HER2.CAR-t-haNK cells are shown in the graph of FIG. 43.

### Example 8: CD30-CAR with FcϵRIγ Signaling Domain

In this example, the inventors constructed a 1^{st} generation CARs with a FcϵRIγ signaling domain that included an anti-CD30 scFv coupled to a CD8 hinge, that in turn was coupled to a CD28 transmembrane domain, which was coupled to a FcϵRIγ signaling domain. The so constructed CD30-CAR had a nucleic acid sequence of SEQ ID NO:61.

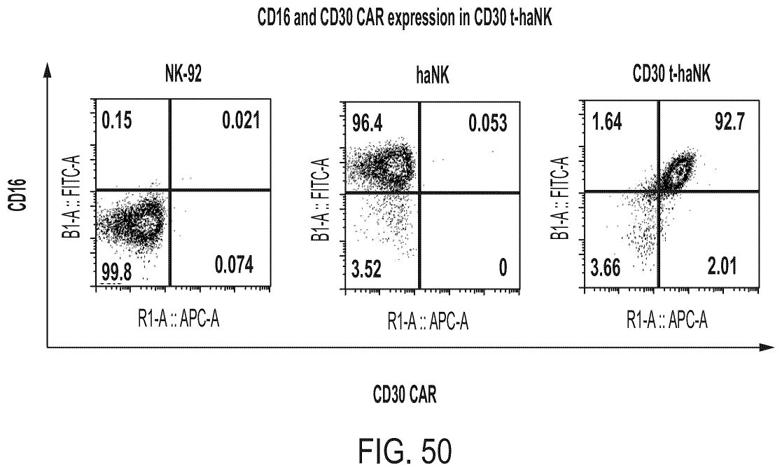

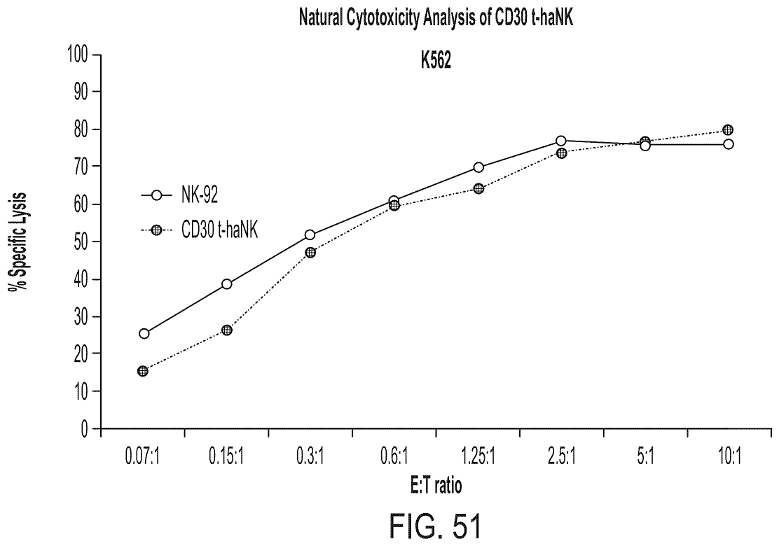

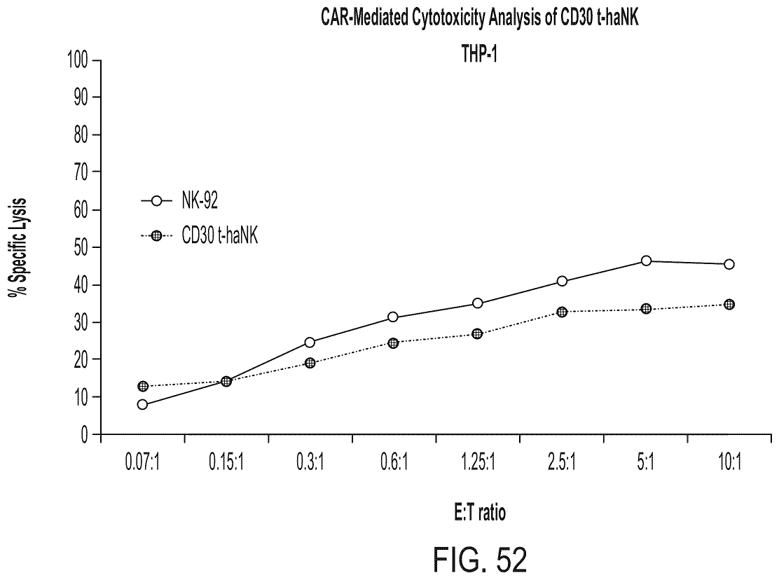

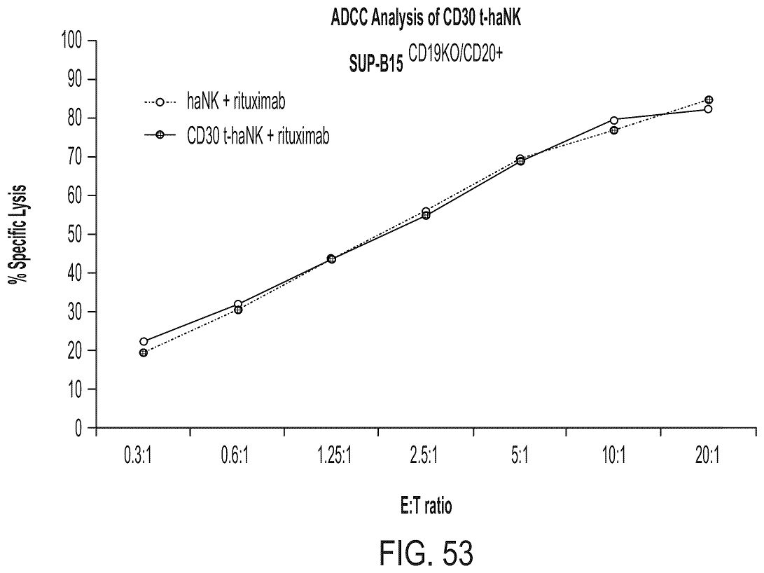

Expression of the CD30-CAR is demonstrated in the results of FIG. 50, while the results for natural cytotoxicity of the recombinant cells are shown in FIG. 51. CAR mediated cytotoxicity was demonstrated in the results of FIG. 52, while exemplary results for ADCC are shown in the data of FIG. 53.

### Example 9: EGFR-CAR with FcϵRIγ Signaling Domain

In this example, the inventors constructed a P^{t} generation CARs with a FcϵRIγ signaling domain that included an anti-EGFR scFv coupled to a CD8 hinge, that in turn was coupled to a CD28 transmembrane domain, which was coupled to a FcϵRIγ signaling domain. The so constructed EGFR-CAR had a nucleic acid sequence of SEQ ID NO:62.

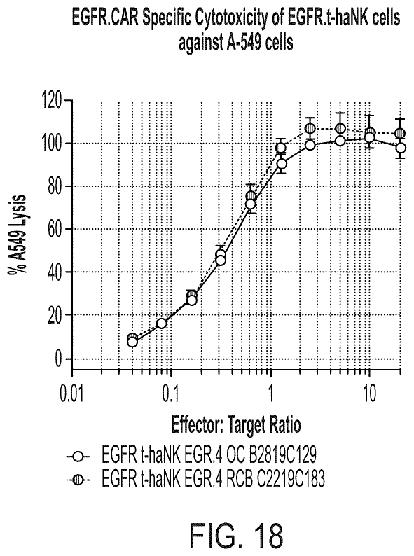

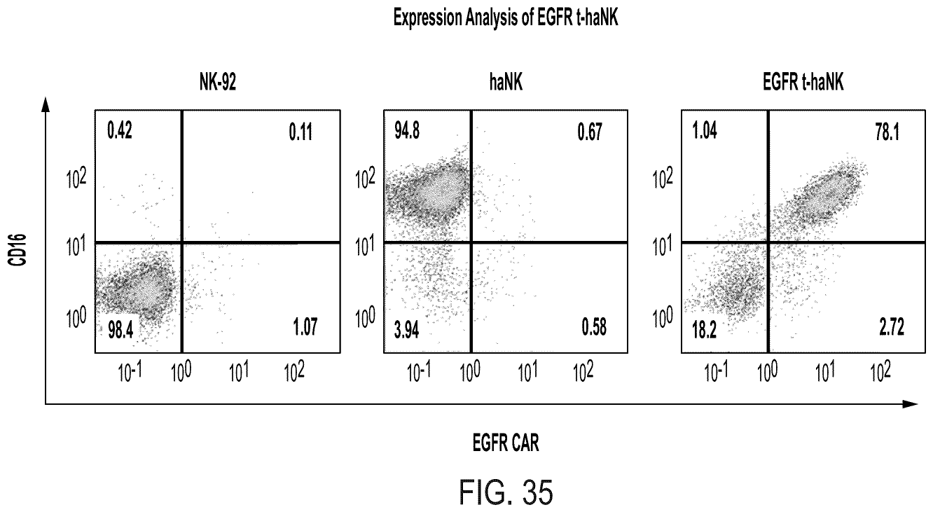

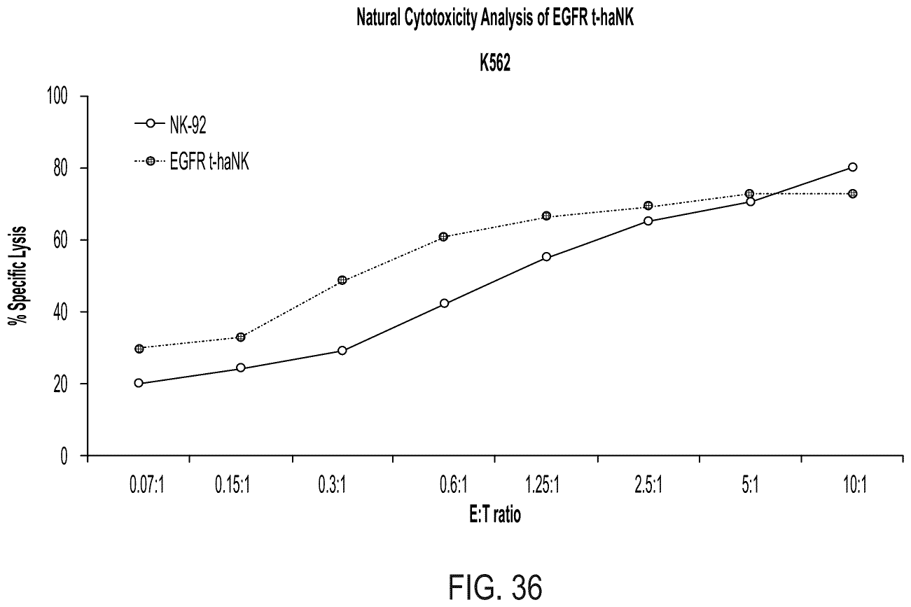

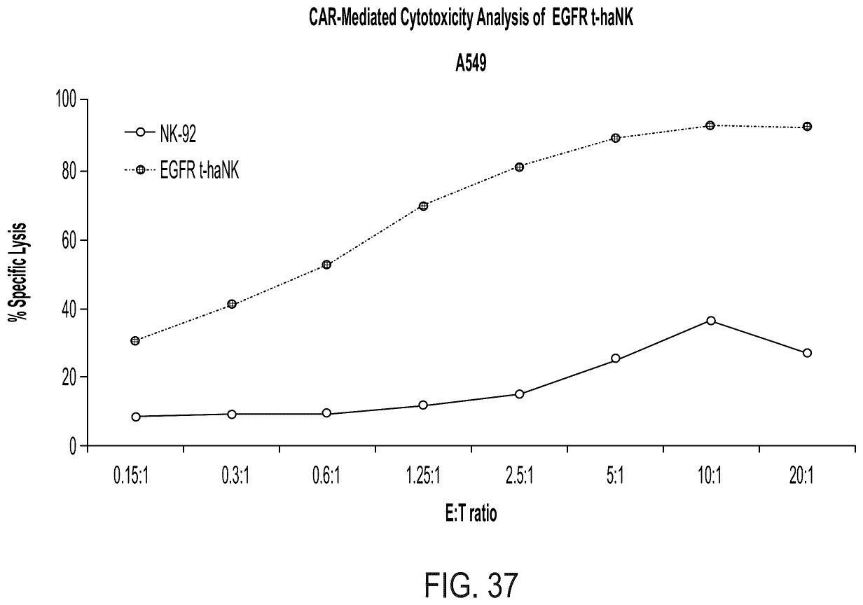

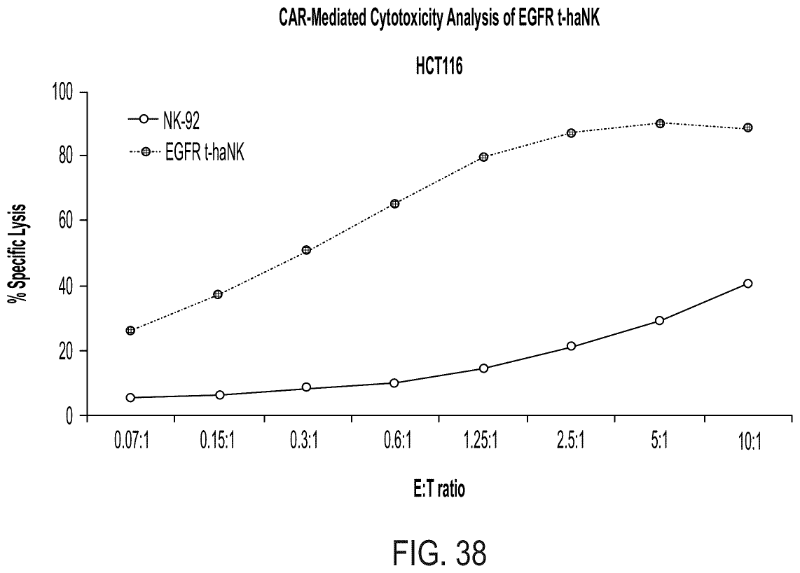

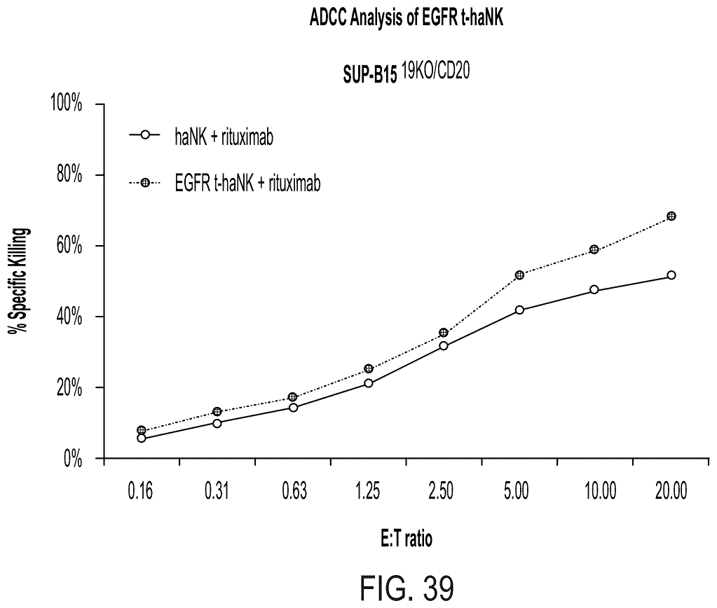

Functionality of the so constructed EGFR.CAR-t-haNK cells was tested against A-549 cells using a standard cytotoxicity assay and exemplary results are shown in FIG. 17. As can be readily seen from the data, the EGFR.CAR-t-haNK cells expressing the CAR with the FcϵRIγ signaling domain exhibited significant cytotoxicity against the A-549 target cells. Expression of the EGFR-CAR in the EGFR.CAR-t-haNK cells is shown in FIG. 35, while natural cytotoxicity results are shown in FIG. 36. Exemplary results for CAR mediated cytotoxicity of EGFR.CAR-t-haNK cells are shown in FIG. 37 and FIG. 38, while results for ADCC of EGFR.CAR-t-haNK cells are shown in FIG. 39.

### Example 10: IGF1R-CAR with FcϵRIγ Signaling Domain

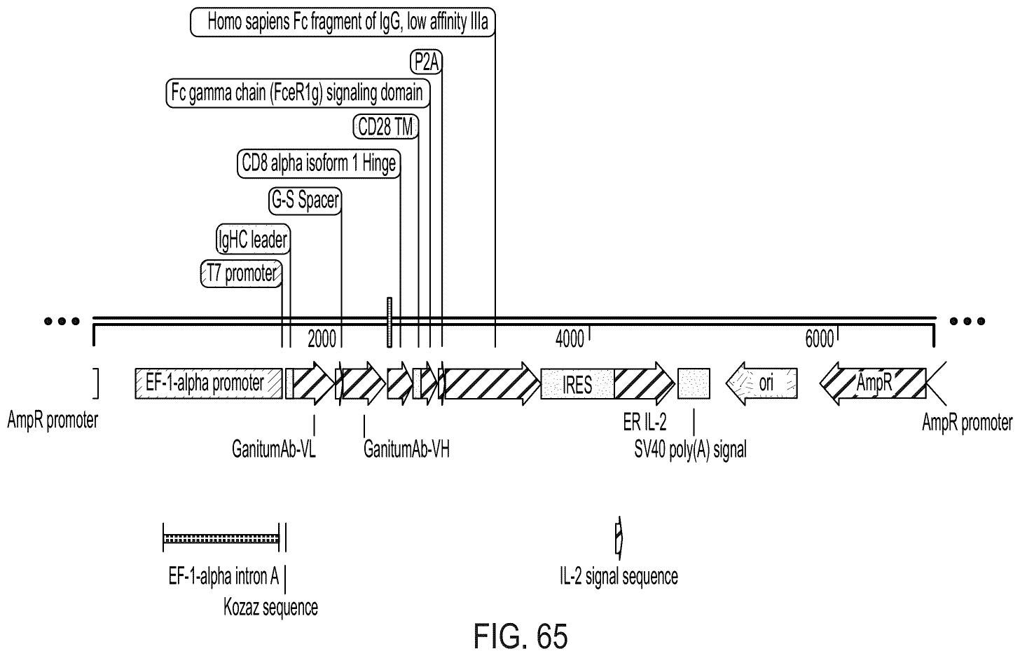

In this example, the inventors constructed a 1^{st} generation CARs with a FcϵRIγ signaling domain that included an anti-IGF1R scFv coupled to a CD8 hinge, that in turn was coupled to a CD28 transmembrane domain, which was coupled to a FcϵRIγ signaling domain. The so constructed IGF1R-CAR had a nucleic acid sequence of SEQ ID NO:63, and a tricistronic construct encoding IGF1R-CAR, CD16, and IL-2^{ER} had a nucleic acid sequence of SEQ ID NO:76, which is also schematically illustrated in Fig.65.

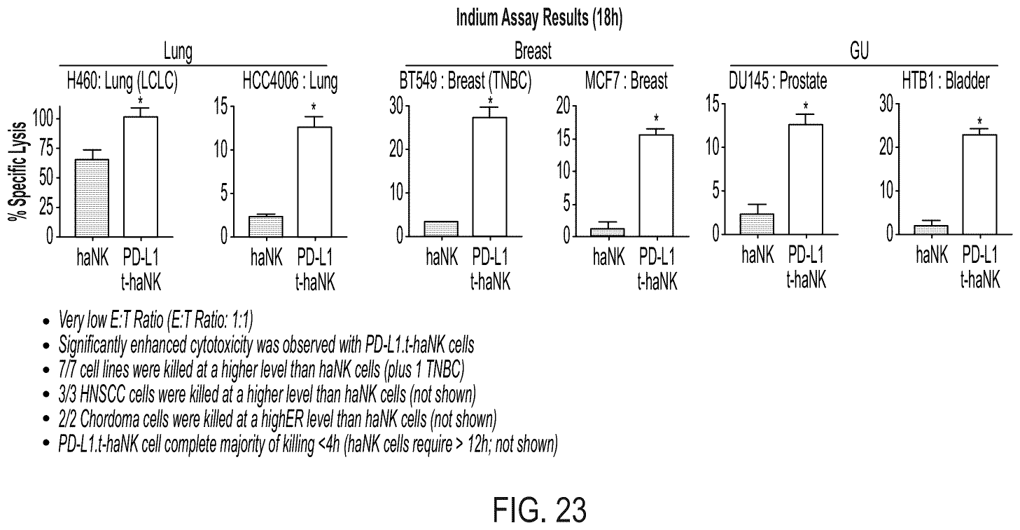

Functionality of the so constructed IGF1R.CAR-t-haNK cells was tested against MDA-MB-231 cells using a standard cytotoxicity assay in comparison with a 2^{nd} generation CAR (CD28/CD3z) and exemplary results are shown in FIG. 22. As can be readily seen from the data, the IGF1R.CAR-t-haNK cells expressing the CAR with the FcϵRIγ signaling domain exhibited significant and target specific cytotoxicity against the MDA-MB-231 target cells, which was comparable with the cytotoxicity of the 2^{nd} generation CAR.

### Example 11: CD123-CAR with FcϵRIγ Signaling Domain

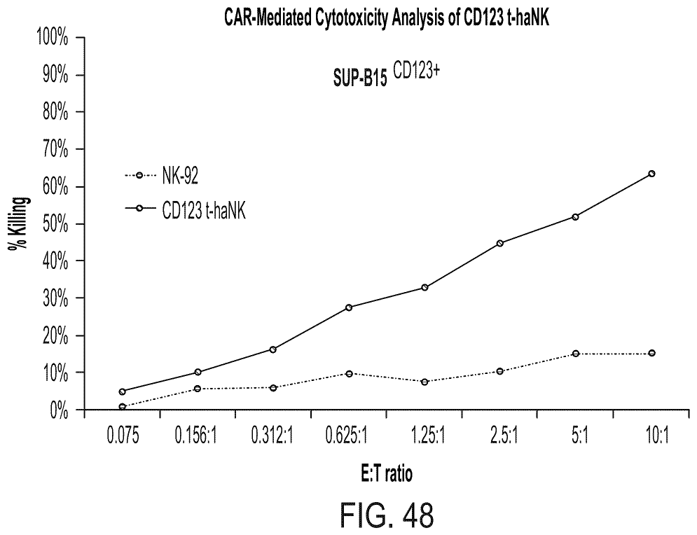

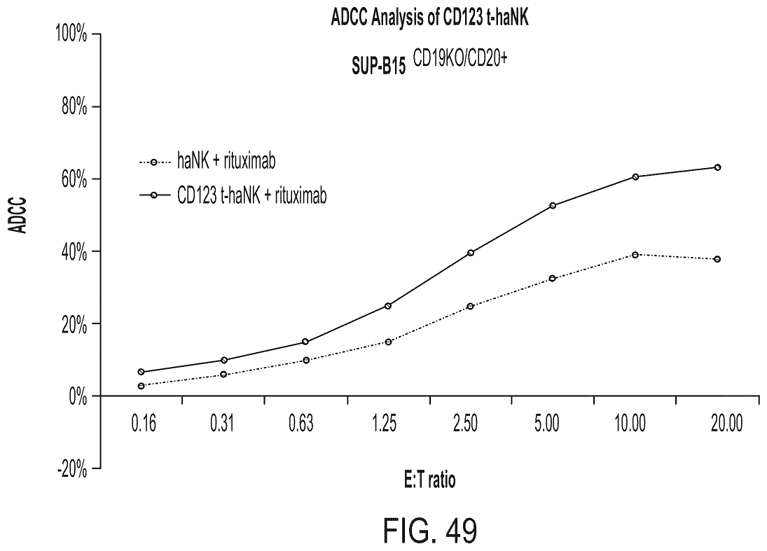

In this example, the inventors constructed a 1^{st} generation CARs with a FcϵRIγ signaling domain that included an anti-CD123 scFv coupled to a CD8 hinge, that in turn was coupled to a CD28 transmembrane domain, which was coupled to a FcϵRIγ signaling domain. The so constructed CD123-CAR had a nucleic acid sequence of SEQ ID NO:64. Data for the CAR mediated cytotoxicity of the CD123-CAR expressing recombinant NK cells is shown in FIG. 48, and FIG. 49 shows exemplary data for ADCC of CD123-CAR expressing recombinant NK cells.

### Example 12: PD-L1-CAR with FcϵRIγ Signaling Domain

In this example, the inventors constructed a 1^{st} generation CARs with a FcϵRIγ signaling domain that included an anti-PD-L1 scFv coupled to a CD8 hinge, that in turn was coupled to a CD28 transmembrane domain, which was coupled to a FcϵRIγ signaling domain. The so constructed PD-L1-CAR had a nucleic acid sequence of SEQ ID NO:65.

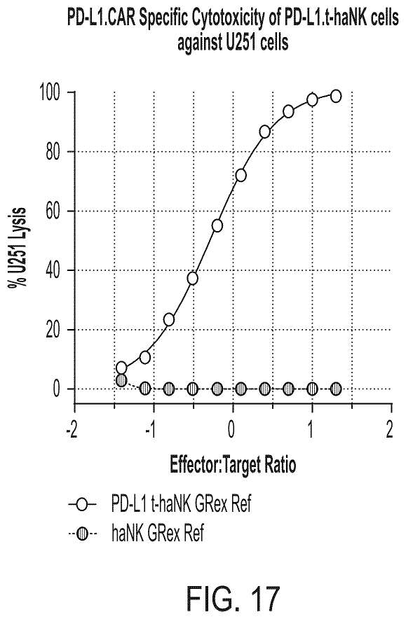

Functionality of the so constructed PD-L1.CAR-t-haNK cells was tested against SUP-B15.PD-L1⁺ cells using a standard cytotoxicity assay and exemplary results are shown in FIG. 16. As can be readily seen from the data, the PD-L1.CAR-t-haNK cells expressing the CAR with the FcϵRIγ signaling domain exhibited significant cytotoxicity against the SUP-B15.PD-L1⁺ target cells.

Functionality of the so constructed PD-L1.CAR-t-haNK cells was also tested against U251 cells using a standard cytotoxicity assay and exemplary results are shown in FIG. 17 along with non-transfected haNK cells. As can be readily seen from the data, the PD-L1.CAR-t-haNK cells expressing the CAR with the FcϵRIγ signaling domain exhibited target specific and significant cytotoxicity against the U251 target cells, whereas the haNK control cells had substantially no cytotoxicity against the same U251 cells.

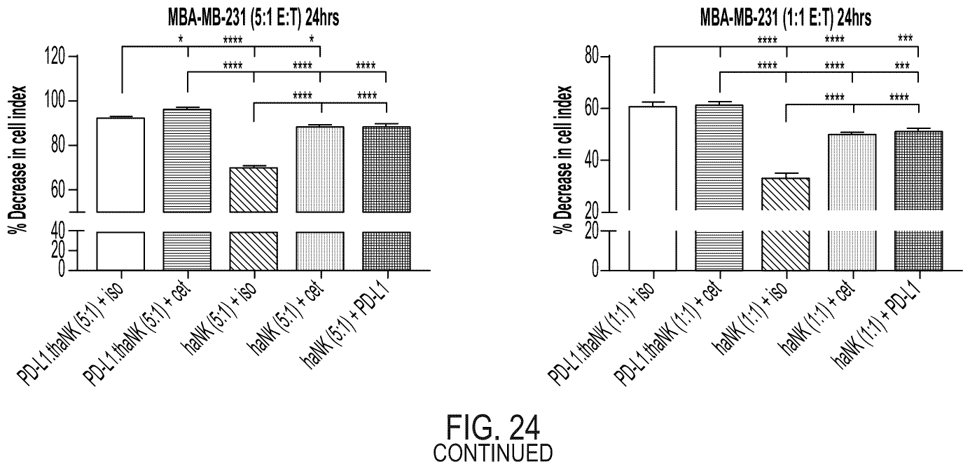

In still further experiments on target cell specificity with respect to PD-L1, the inventors tested several PD-L1 positive tumor cell lines using the PD-L1.CAR-t-haNK cells along with haNK cells as control for general cytotoxicity. As can be readily seen from FIG. 24, the PD-L1.CAR-t-haNK cells had superior cytotoxicity across a wide variety of tumor cells (lung, breast, genitury tumor cells, and additionally, head and neck small cell cancer, chordoma). Notably, the PD-L1.CAR-t-haNK cells required less than 4 hours for the majority (>85%) of cell killing whereas the control haNK cells required more than 12 hours.

FIG. 24 further illustrates cytotoxicity of the PD-L1.CAR-t-haNK cells against MDA-MB-231 cells as compared to various other control cells (haNK cells as indicated). As can be taken from the data, at a 5:1 E:T ratio, MDA-MB-231 lysis by PD-L1.thaNK was improved by cetuximab, and haNK activity was improved by the addition of cetuximab and a-PD-L1. Plain PD-L1.thank had improved cytotoxic activity compared to haNK and haNK+cetuximab, and plain PD-L1.thank killing was comparable to that of haNK+PD-L1 antibody but PD-L1.thank+cetuximab outperformed haNK+cetuximab and haNK+PD-L1. At a 1:1 E:T ratio, PD-L1.thaNK activity was the same with or without cetuximab, and PD-L1.thaNK significantly outperformed intrinsic and ADCC-mediated killing by hank. haNK activity was improved by the addition of cetuximab and a-PD-L1.

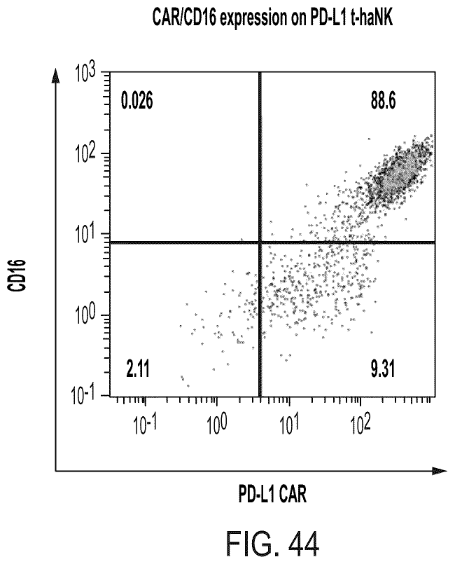

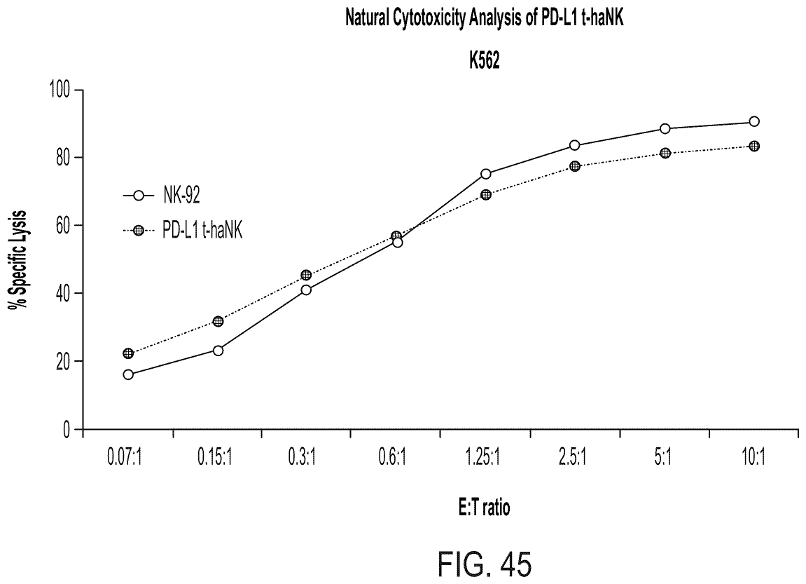

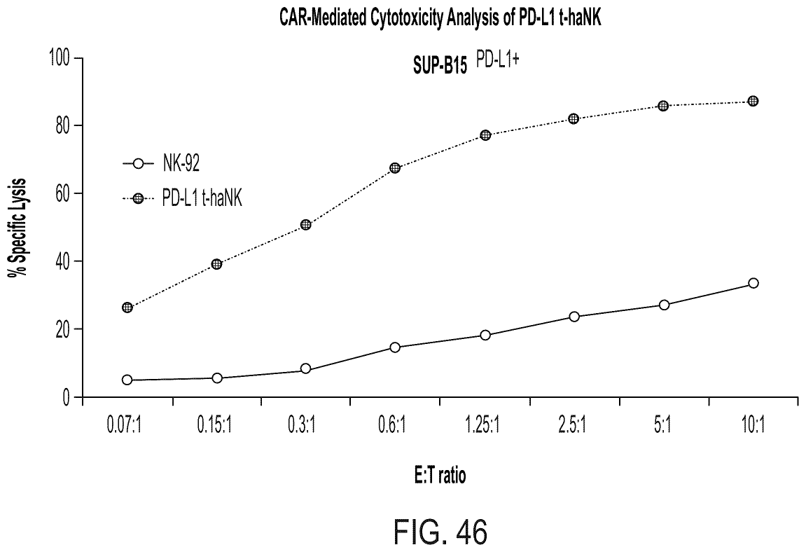

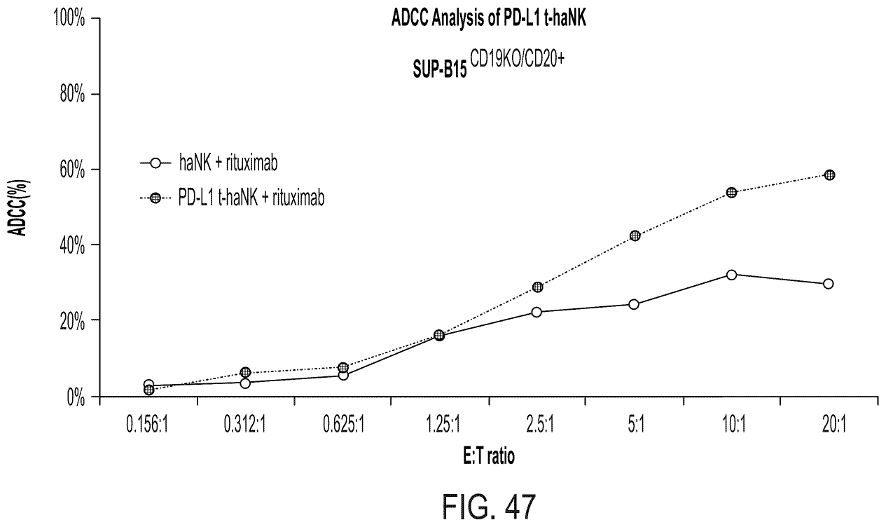

In further experiments, the inventors demonstrated expression of the PD-L1.CAR in PD-L1.CAR-t-haNK cells as is illustrated in FIG. 44. Natural cytotoxicity of the PD-L1.CAR-t-haNK cells is shown in the results of FIG. 45, while results for CAR mediated cytotoxicity are shown in FIG. 46. Exemplary data for ADCC of PD-L1.CAR-t-haNK cells are shown in the graph of FIG. 47.

### Example 13: CD33-CAR with FcϵRIγ Signaling Domain

In this example, the inventors constructed a 1^{st} generation CARs with a FcϵRIγ signaling domain that included an anti-HER2 scFv coupled to a CD8 hinge, that in turn was coupled to a CD28 transmembrane domain, which was coupled to a FcϵRIγ signaling domain. The so constructed CD33.CAR had a nucleic acid sequence of SEQ ID NO:66.

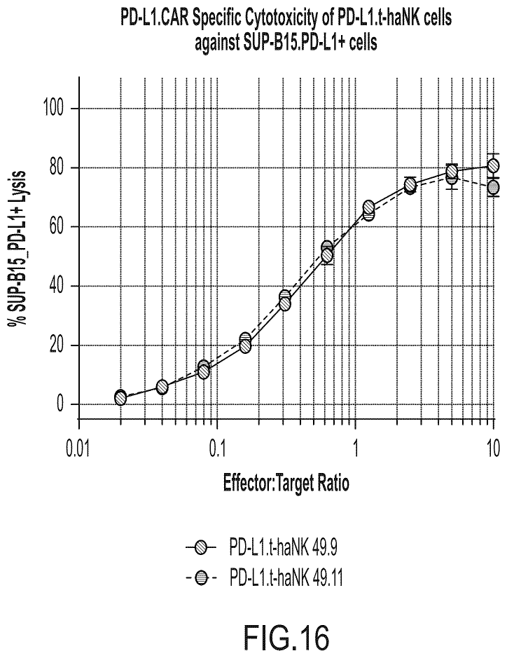

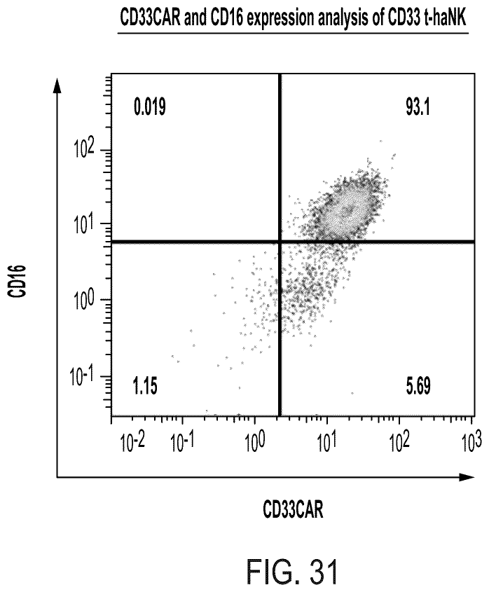

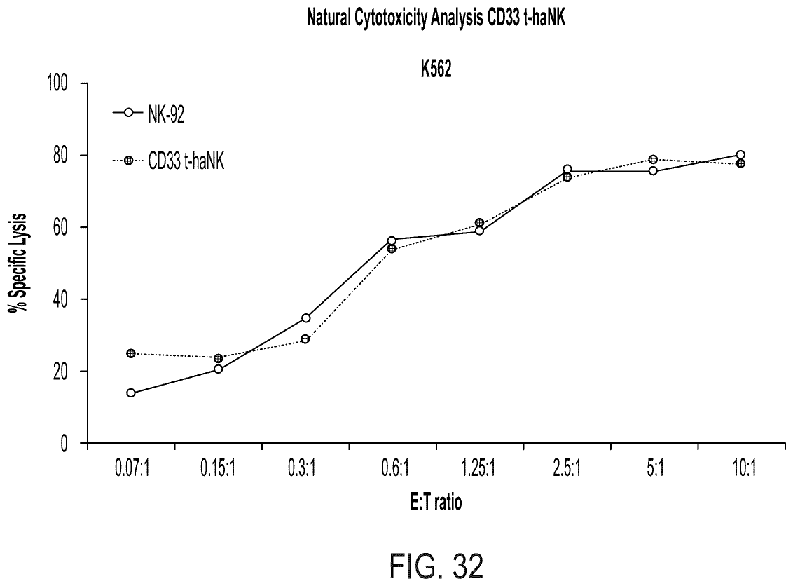

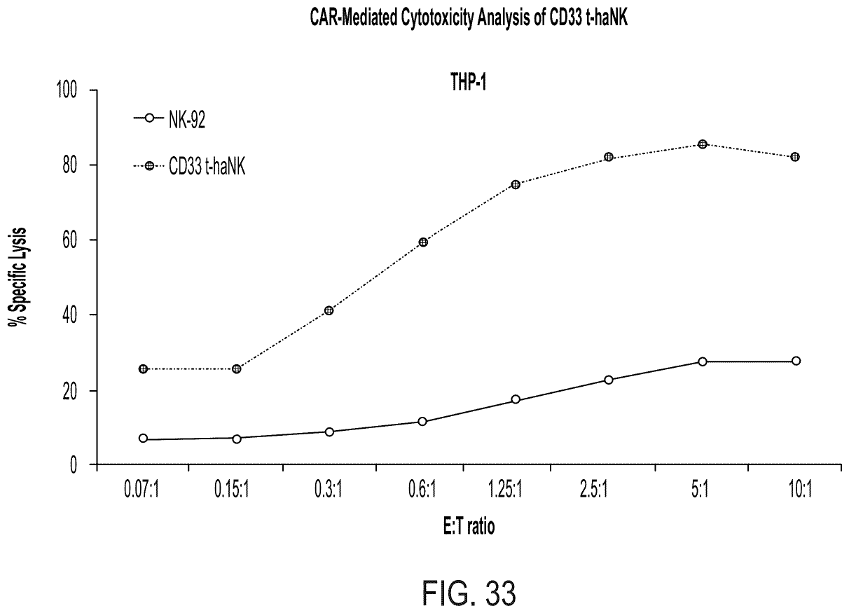

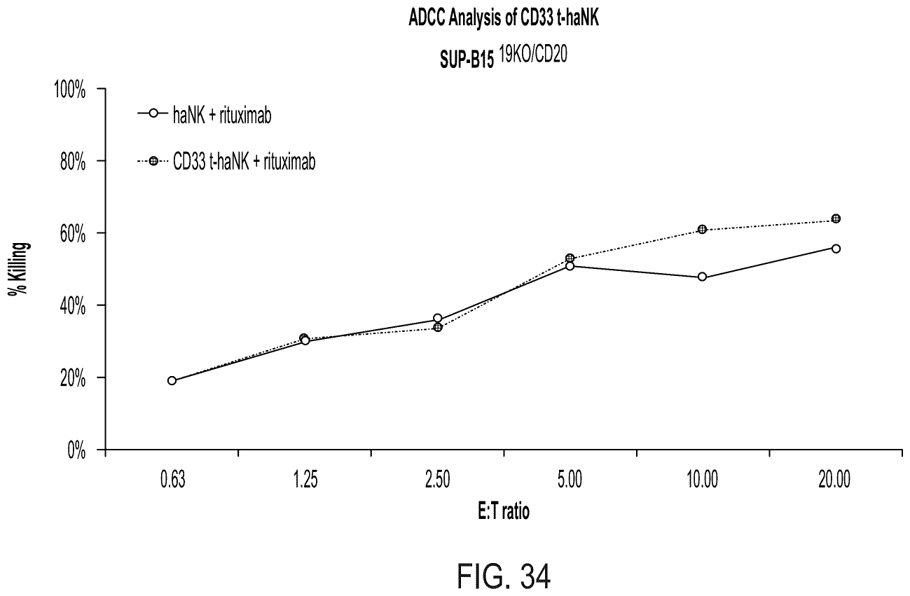

Functionality of the so constructed CD33.CAR-t-haNK cells was tested against THP-1 cells using a standard cytotoxicity assay and exemplary results are shown in FIG. 15. As can be readily seen from the data, the CD33.CAR-t-haNK cells expressing the CAR with the FcϵRIγ signaling domain exhibited significant cytotoxicity against the THP-1 target cells. Further data depicting strong expression of the CD33CAR in NK-92 cells are presented in FIG. 31. Natural cytotoxicity of the CD33.CAR-t-haNK cells against K562 cells is shown in FIG. 32, and FIG. 33 depicts results for CAR mediated cytotoxicity against THP-1 cells. FIG. 34 shows further results for ADCC of CD33.CAR-t-haNK cells against SUP-B15 CD19^{KO}/CD20⁺ with rituximab.

### Example 14: gp120-CAR with FcϵRIγ Signaling Domain

In this example, the inventors constructed a 1^{st} generation CARs with a FcϵRIγ signaling domain that included an anti-gp120 scFv coupled to a CD8 hinge, that in turn was coupled to a CD28 transmembrane domain, which was coupled to a FcϵRIγ signaling domain. The so constructed gp120-CAR had a nucleic acid sequence of SEQ ID NO:67.

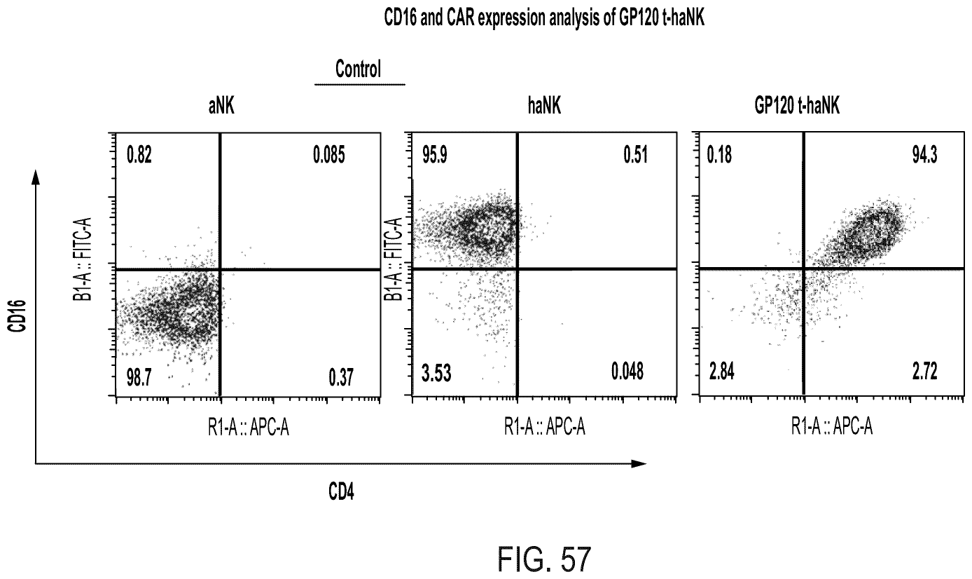

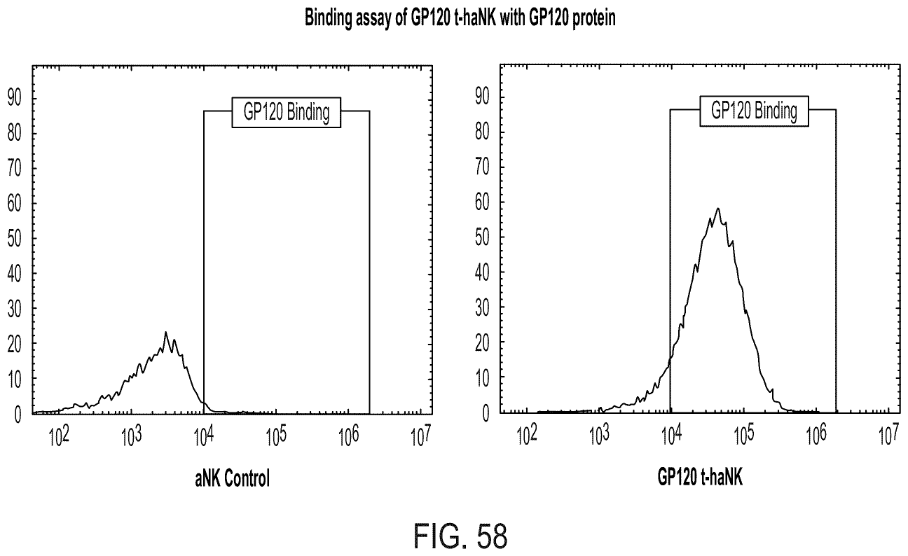

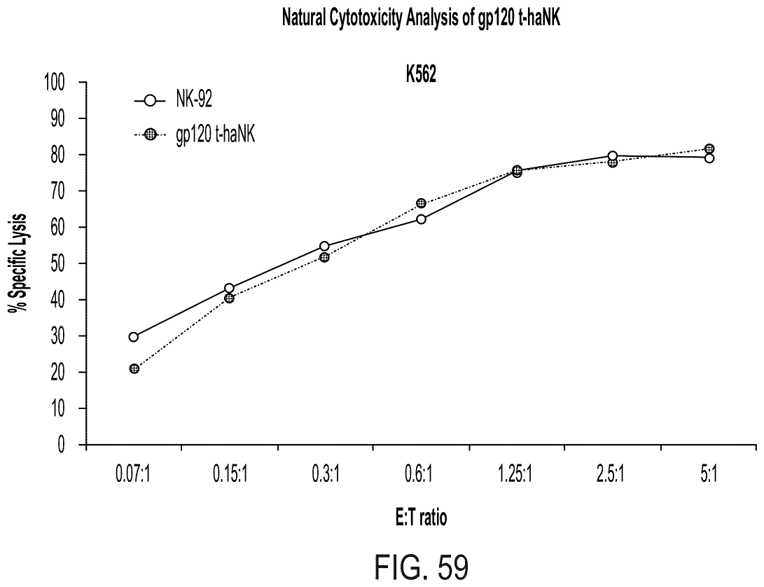

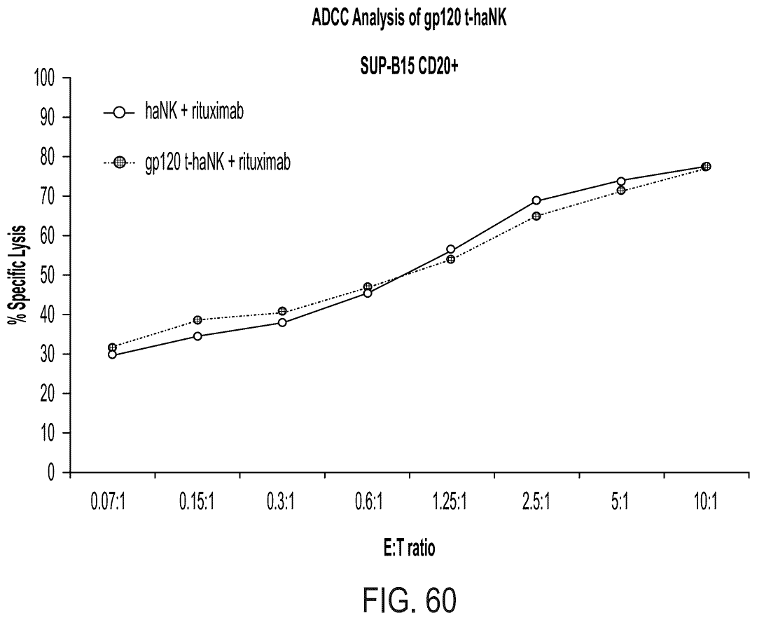

The inventors further demonstrated that so generated cells expressed significant quantities of CD16 and gp120CAR as can be seen from FIG. 57. Binding of GP120 to the gp120CAR was shown as demonstrated in FIG. 58 versus non-recombinant aNK cells as negative control. Natural cytotoxicity of the so generated cells is shown in FIG. 59, while corresponding ADCC data are shown in FIG. 60.

### Example 15: B7-H4-CAR with FcϵRIγ Signaling Domain

In this example, the inventors constructed a 1^{st} generation CARs with a FcϵRIγ signaling domain that included an anti-B7-H4 scFv coupled to a CD8 hinge, that in turn was coupled to a CD28 transmembrane domain, which was coupled to a FcϵRIγ signaling domain. The so constructed B7-H4-CAR had a nucleic acid sequence of SEQ ID NO:68.

### Example 16: BCMA-CAR with FcϵRIγ Signaling Domain

In this example, the inventors constructed a 1^{st} generation CARs with a FcϵRIγ signaling domain that included an anti-BCMA scFv coupled to a CD8 hinge, that in turn was coupled to a CD28 transmembrane domain, which was coupled to a FcϵRIγ signaling domain. The so constructed BCMA-CAR had a nucleic acid sequence of SEQ ID NO:69.

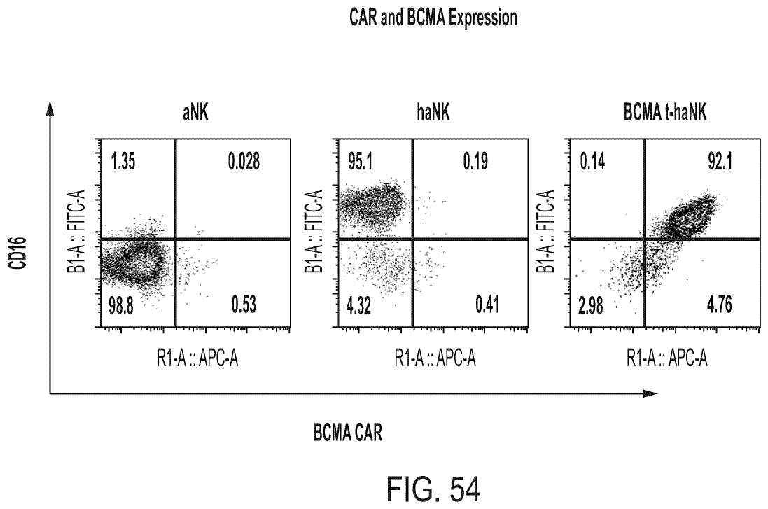

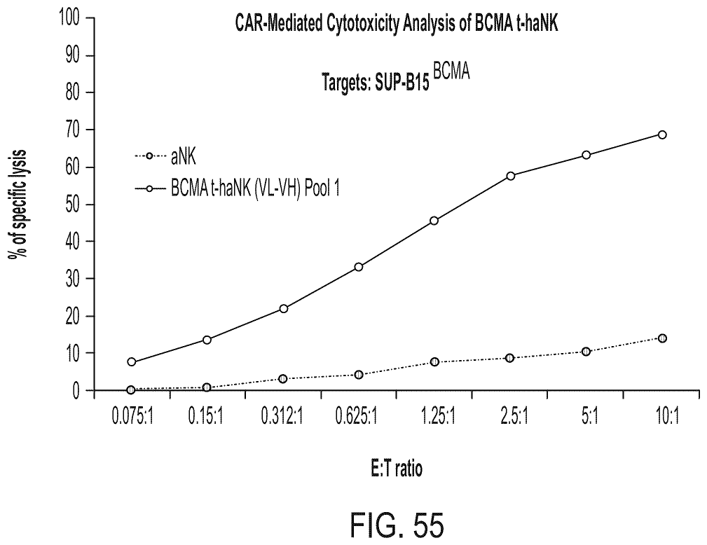

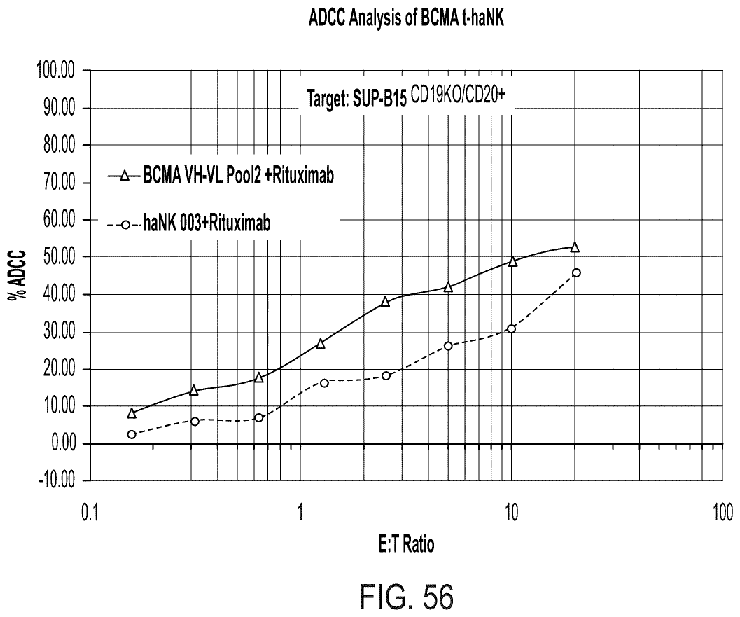

BCMA expression was confirmed as is shown in the exemplary results of FIG. 54, and CAR mediated cytotoxicity was demonstrated against target cells as is shown in FIG. 55. Similarly, as can be seen from the results in FIG. 56, recombinant cells had significant ADCC using rituximab as antibody against the target cells.

### Example 17: GD2-CAR with FcϵRIγ Signaling Domain

In this example, the inventors constructed a P^{t} generation CARs with a FcϵRIγ signaling domain that included an anti-GD2 scFv coupled to a CD8 hinge, that in turn was coupled to a CD28 transmembrane domain, which was coupled to a FcϵRIγ signaling domain. The so constructed GD2-CAR had a nucleic acid sequence of SEQ ID NO:70.

Example 18: FAP-CAR with FcϵRIγ Signaling Domain

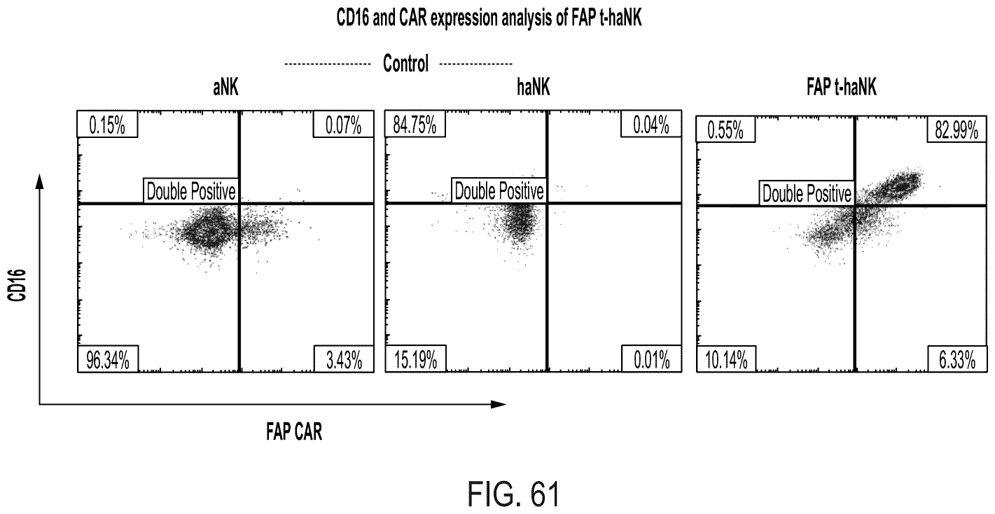

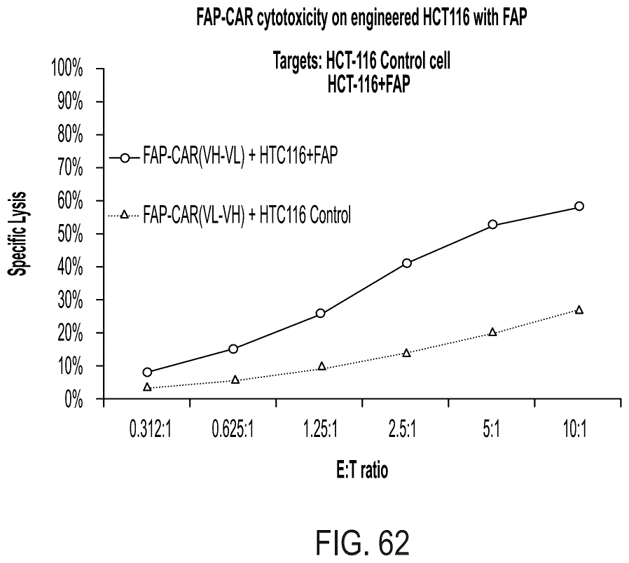

In this example, the inventors constructed a 1^{st} generation CARs with a FcϵRIγ signaling domain that included an anti-FAP scFv coupled to a CD8 hinge, that in turn was coupled to a CD28 transmembrane domain, which was coupled to a FcϵRIγ signaling domain. The so constructed FAP-CAR had a nucleic acid sequence of SEQ ID NO:71. Expression of the FAP-CAR is shown in the data of FIG. 61, and FAP.CAR cytotoxicity is demonstrated on target cells in the results of FIG. 62.

Example 19: CD20-CAR with FcϵRIγ Signaling Domain

In this example, the inventors constructed a 1^{st} generation CARs with a FcϵRIγ signaling domain that included an anti-CD20 scFv coupled to a CD8 hinge, that in turn was coupled to a CD28 transmembrane domain, which was coupled to a FcϵRIγ signaling domain. The so constructed CD20-CAR had a nucleic acid sequence of SEQ ID NO:74.

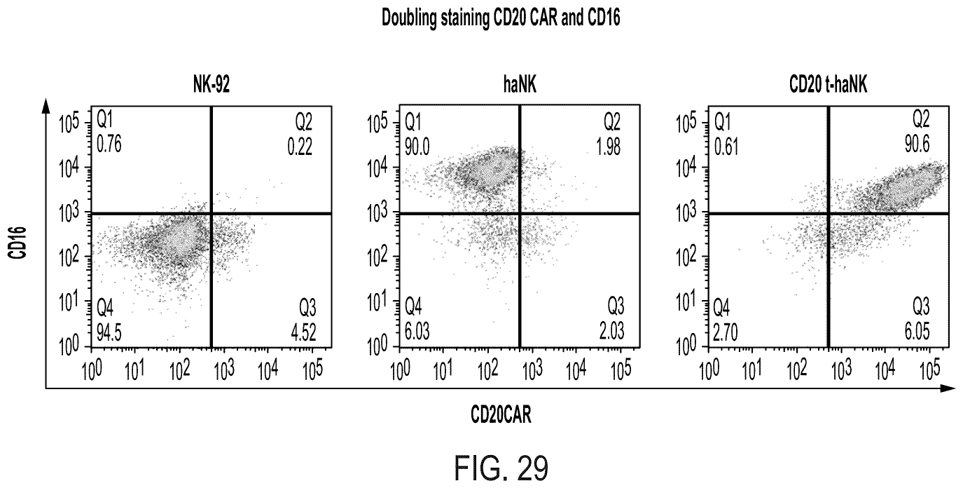

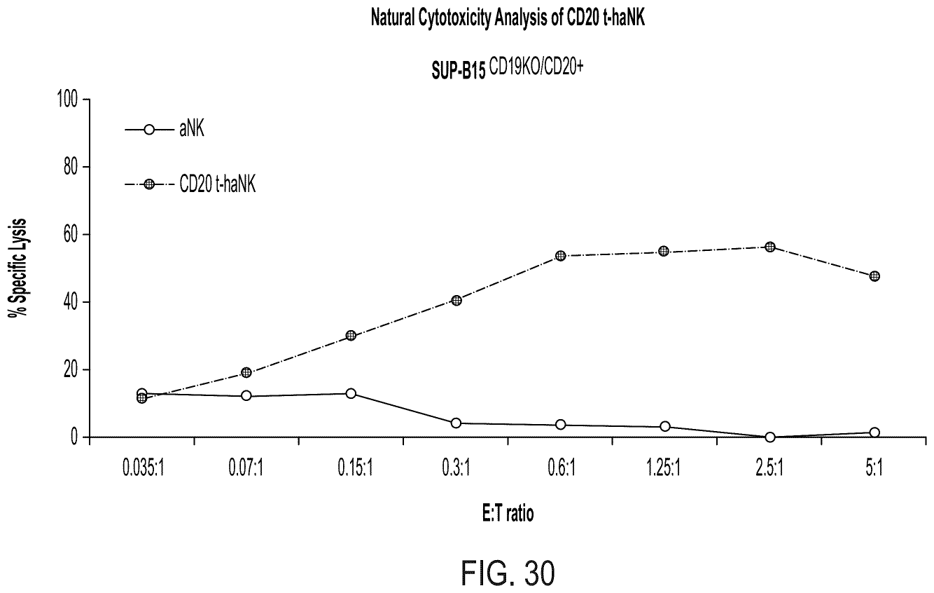

Expression of the CD20 CAR in NK-92 cells is shown in the results of FIG. 29. As can be readily seen, CD20.CAR is expressed strongly in the vast majority of recombinant cells (along with CD16 from the linearized DNA as noted above). FIG. 30 depicts exemplary results for cytotoxicity of the CD20.CAR NK cells against CD20⁺ target cells.

Example 20: CSPG-4-CAR with FcϵRIγ Signaling Domain

In this example, the inventors constructed a 1^{st} generation CARs with a FcϵRIγ signaling domain that included an anti-CSPG-4 scFv coupled to a CD8 hinge, that in turn was coupled to a CD28 transmembrane domain, which was coupled to a FcϵRIγ signaling domain. The so constructed CSPG-4-CAR had a nucleic acid sequence of SEQ ID NO:75. Expression of the CSPG-4-CAR was confirmed with FACS analysis and exemplary results are shown in

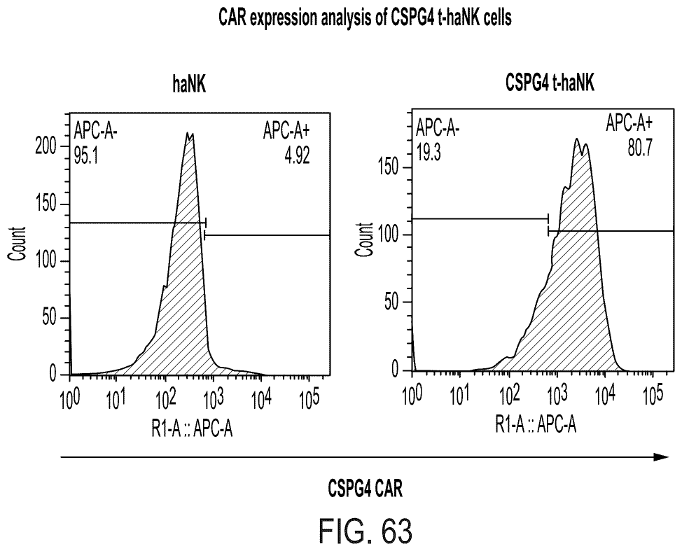

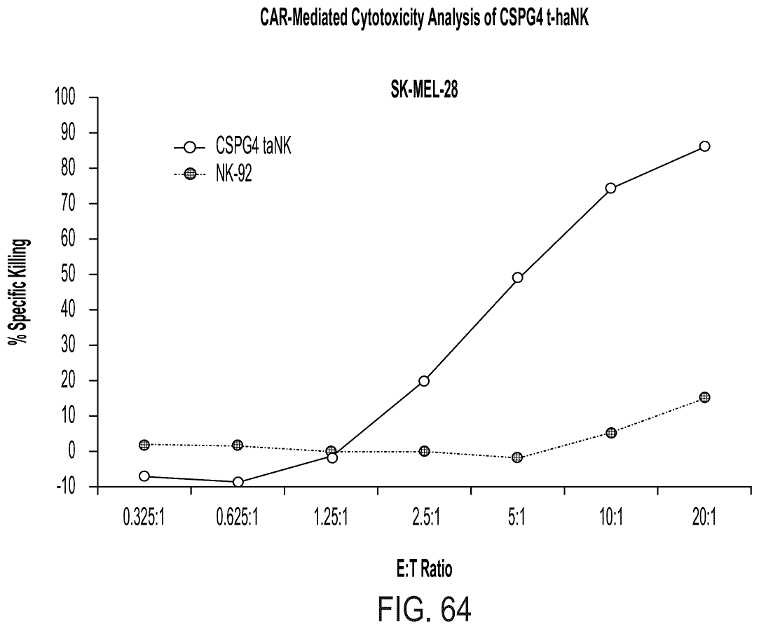

FIG. 63. Thusly constructed cells also exhibited significant cytotoxicity as is shown in the exemplary data of FIG. 64.

### Example 21: CD19-CAR with FcϵRIγ Signaling Domain

In this example, the inventors used the 1^{st} generation CARs as described above having a FcϵRIγ signaling domain that included an anti-CD19 scFv coupled to a CD8 hinge, that in turn was coupled to a CD28 transmembrane domain, which was coupled to a FcϵRIγ signaling domain and transfected NK-92cells with linearized DNA for functional testing.

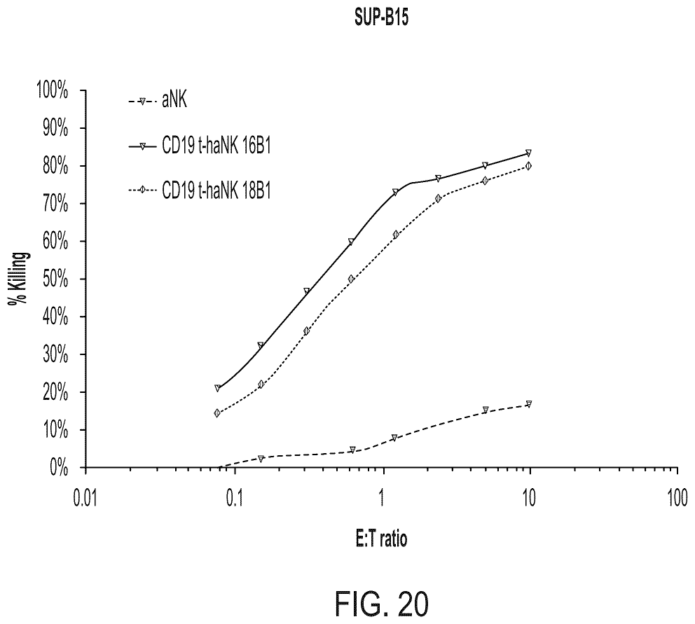

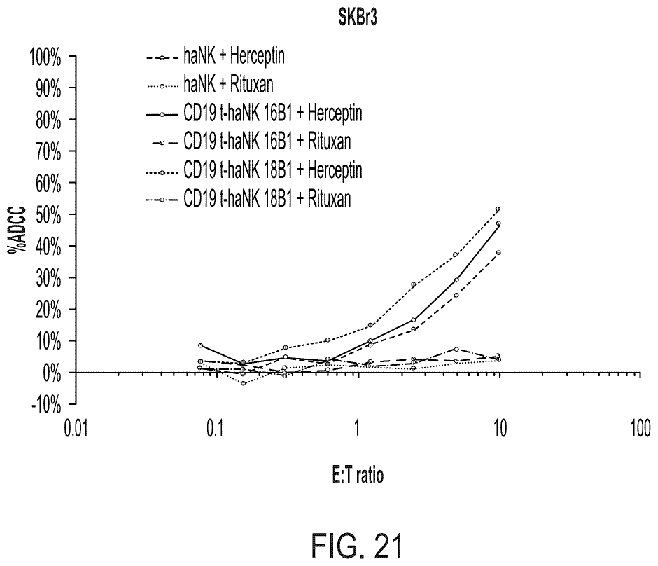

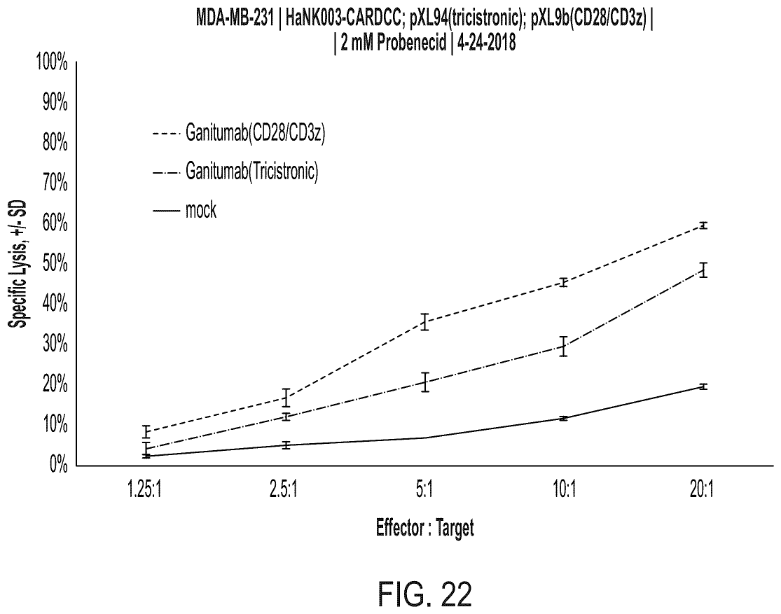

Functionality of the so constructed CD19.CAR-t-haNK cells was tested against K562 cells for determination of general cytotoxicity using a standard cytotoxicity assay and exemplary results are shown in Fig.19. As can be readily seen, the CD19.CAR-t-haNK cells expressing the CAR with the FcϵRIγ signaling domain exhibited significant cytotoxicity against the K562 target cells. In a further set of experiments, target specific cytotoxicity was determined using SUP-B15 cells in comparison with aNK cells as control, and exemplary results are shown in FIG. 20. Once more, CD19.CAR-t-haNK cells expressing the CAR with the FcϵRIγ signaling domain exhibited significant and target specific cytotoxicity. In yet another set of experiments, target specific ADCC was determined using SKBr3 cells using Herceptin and Rituxan as antibodies, and exemplary results are shown in FIG. 21. Again, CD19.CAR-t-haNK cells expressing the CAR with the FcϵRIγ signaling domain exhibited significant antibody and target specific ADCC. Notably, doubling times of the recombinant NK cells were substantially the same as aNK cells.

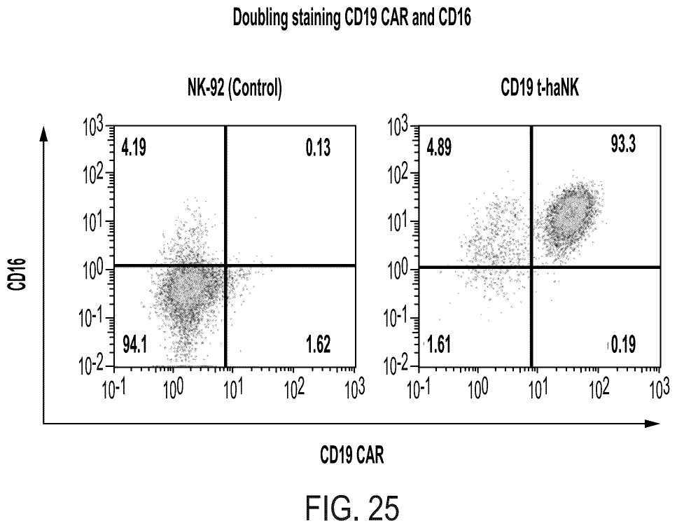

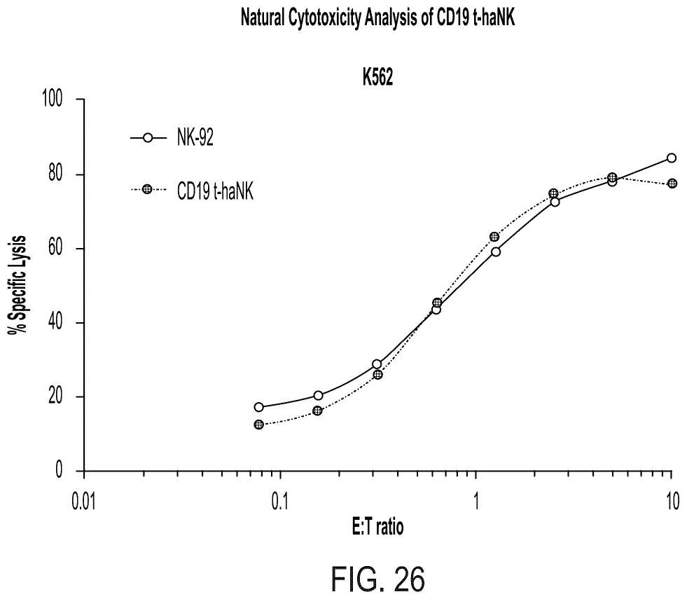

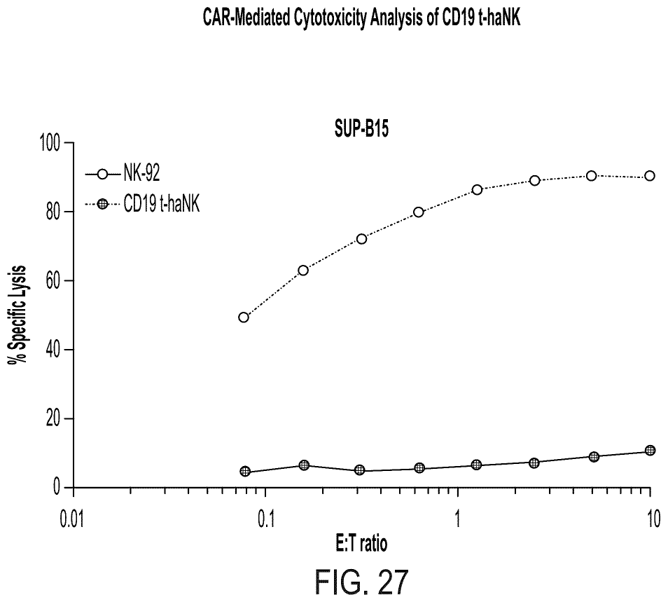

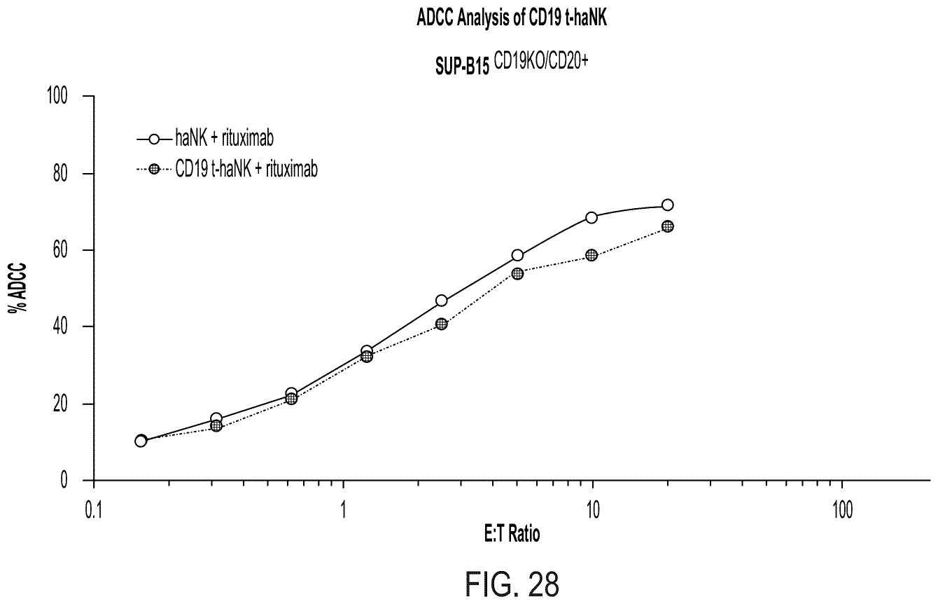

FIG. 25 exemplarily illustrates CD19.CAR expression from linearized DNA that included a segment encodingCD16 and IL-2^{ER} in NK-92 cells versus control. As can be seen form FIG. 25, the expression was very strong across the vast majority of cells. Additional results for natural cytotoxicity of CD19.CAR t-haNK cells against K562 cells and targeted cytotoxicity against SUP-B15 cells are depicted in FIG. 26 and FIG. 27. Exemplary further results for ADCC of CD19.CAR t-haNK cells against SUP-B15CD19^{KO}/CD20⁺ cells are shown in FIG. 28.

### Example 22: Anti-Tumor Activity of PD-L1-Targeting t-haNK Cells in Human Xenograft Models in NSG Mice

MDA-MB-231 and HCC827 were used as validated xenograft models that are PD-L1 positive, and efficacy of PD-L1 t-haNK cells in varied formulations, dosing levels, and dosing routes (IV and IT) was evaluated.

Animals: Animal type: NSG mice (JAX), females, 9-10 weeks old; Number of animals for MDA-MB-231 model: 24 (fresh cells), and for HCC827 model: 24 (fresh cells)+6 (cryopreserved cells). Tumor model used the following cell line: MDA-MB-231 (human breast adenocarcinoma) and HCC827 (human lung adenocarcinoma), Route of inoculation was subcutaneous on both flanks, and average tumor burden upon treatment initiation was for MDA-MB-231 about 100 mm³ and for HCC827 about 75-80 mm³.

Treatment articles: Anti-PD-L1 t-haNK, freshly prepared, irradiated, at a concentration: 5E7 cells/mL or 2E7 cells/mL; Vehicle control was X-VIVO™ 10 medium; Method of administration was IV and IT as noted. Dosage for IV NK dosing was 1E7 cells/dose in 200 μL (Freshly prepared cells), 4E6 cells/dose in 200 μL (Cryopreserved cells); for IT NK dosing (fresh cells only) dose was 2.5E6 cells/tumor/dose in 50 μL. Dosing frequency was Twice a week (M/Th or T/F) for 4 consecutive weeks, and first day of dosing was defined as Day 1.

Study design for MDA-MB-231 is in Table 4 below (This study was ended on Day 27, when some animals in Groups A, C and D had reached combined tumor volume of >2000 mm³)

TABLE 4

| | | Tumor | | Fresh or | NK Cell | NK dosing | Treatment | Dosing |
|---|---|---|---|---|---|---|---|---|
| Group | N | model | Treatment | Frozen | Dose | route | Regimen | Volume |
| A | 6 | MDA-MB-231 | Vehicle | / | / | IV | BIW × 4 | 200 μL |
| | | SC, bilateral | | | | | weeks | |
| B | 6 | 1 × 10⁶ | PD-L1 | Fresh | 1E7 | IV | BIW × 4 | 200 μL |
| | | | t-haNK | | | | weeks | |
| C | 6 | | Vehicle | / | / | IT | BIW × 4 | 50 μL |
| | | | | | | | weeks | |
| D | 6 | | PD-L1 | Fresh | 2.5E6 | IT | BIW × 4 | 50 μL |
| | | | t-haNK | | | | weeks | |

Study design for HCC827 is in Table 5 below (This study was ended on Day 29, when surviving animals were re-purposed and transferred to another study).

TABLE 5

| | | Tumor | | Fresh or | NK Cell | NK dosing | Treatment | Dosing |
|---|---|---|---|---|---|---|---|---|
| Group | N | model | Treatment | Frozen | Dose | route | Regimen | Volume |
| A | 6 | HCC827 | Vehicle | / | / | IV | BIW × 4 | 200 μL |
| | | SC, bilateral | | | | | weeks | |
| B | 6 | 1 × 10⁶ | PD-L1 | Fresh | 1E7 | IV | BIW × 4 | 200 μL |
| | | | t-haNK | | | | weeks | |
| C | 6 | | Vehicle | / | / | IT | BIW × 4 | 50 μL |
| | | | | | | | weeks | |
| D | 6 | | PD-L1 | Fresh | 2.5E6 | IT | BIW × 4 | 50 μL |
| | | | t-haNK | | | | weeks | |
| Pilot | 6 | | PD-L1 | Frozen | 4E6 | IV | BIW × 4 | 200 μL |
| | | | t-haNK | | | | weeks | |

Results: Freshly prepared PD-L1 t-haNK cells (1E7 cells/dose) led to marked and long-lasting tumor growth inhibition in both MDA-MB-231 and HCC827 models

MDA-MB-231: tumor stasis: TGI on Day 16: 84% (peak); TGI on Day 26: 79% (last measurement).

HCC827: tumor regression: TGI on Day 16: 120% (peak); TGI on Day 29: 84% (study end).

Cryopreserved PD-L1 t-haNK cells (4E6 cells/dose) also showed statistically significant efficacy in suppressing tumor growth compared to X-VIVO™ 10 media: TGI on Day 26: 60% (peak), and TGI on Day 29: 40% (study end).

Freshly prepared PD-L1 t-haNK cells (1E7 cells/dose) also led to significant reduction of metastatic disease burden in the MDA-MB-231 model as shown in Table 6 below.

TABLE 6

| | | Macroscopic lesions | Overall |
|---|---|---|---|
| Group | Mouse | found in: | Summary |
| A | 1 | Liver, lungs | 100% animals developed |
| (vehicle) | 2 | Ax LNs, liver, lungs | metastases in multiple |
| | 3 | Ax LN (left), liver, | organs |
| | | lungs | |
| | 4 | Liver, lungs | |
| | 5 | Ax LNs, spleen, liver, | |
| | | lungs | |
| | 6 | Ax LNs, liver, lungs | |
| B (PD-L1 | 1 | None | 50% developed metastasis; |
| t-haNK) | 2 | Lungs | all single-organ findings |
| | 3 | Ax LNs | |
| | 4 | None | |
| | 5 | Ax LN (left) | |
| | 6 | None | |

The number of visible nodules in liver was in vehicle: 29±9, in the PD-L1t-haNK group: 0 (P=0.0116 by unpaired 2-tailed t test).

Based on the experiments performed, IV dosing of freshly prepared PD-L1 t-haNK cells at the dosing level of 1E7 cells/dose, twice a week for 4 weeks, showed marked anti-tumor efficacy in both of the subcutaneous xenograft models tested: The treatment resulted in tumor stasis in MDA-MB-231 tumor-bearing mice, with a peak TGI of 84% on Day 16 and an end-of-study TGI of 79% (P<0.0001 for both time points by 2-way ANOVA followed by multiple comparison by Tukey test), and tumor regression in the HCC827 model, with a peak TGI of 120% on Day 16 and an end-of-study TGI of 84% (P<0.0001). IV dosing of cryopreserved PD-L1 t-haNK cells at the dosing level of 4E6 cells/dose, twice a week for 4 weeks, also showed significant therapeutic efficacy in the HCC827 tumor model, reaching a peak TGI of 60% (P<0.0001), and an end-of-study TGI of 40% (P<0.01). IT dosing of freshly prepared PD-L1 t-haNK cells at the dosing level of 2.5E6 cells/dose/tumor, twice a week for 4 weeks, effectively suppressed the growth of HCC827 tumors, resulting in a peak TGI of 70% on Day 20 and an end-of-study TGI of 49% (P<0.001).

Significant adverse reactions were observed for animals that received IV administrations of freshly prepared PD-L1 t-haNK cells (1E7 cells/dose). In contrast to freshly prepared PD-L1 t-haNK cells, cryopreserved cells (dosed at a lower level of 4E6 cells/dose) proved to be safe to the animals after IV administrations. PD-L1 t-haNK cells demonstrated remarkable efficacy in the two subcutaneous tumor models. Cryopreserved cells dosed at the lower 4E6 cells/dose level, also showed significant efficacy in suppressing tumor growth, and proved to be safe for the animals.

Of course, it should be recognized that for all nucleic acid sequences provided herein the corresponding encoded proteins are also expressly contemplated herein. Likewise, for all amino acid sequences, corresponding nucleic acids sequences are also contemplated herein (with any codon usage).

All patent applications, publications, references, and sequence accession numbers cited in the present specification are hereby incorporated by reference in their entirety.

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

It is understood that all numerical values described herein (e.g., pH, temperature, time, concentration, amounts, and molecular weight, including ranges) include normal variation in measurements encountered by one of ordinary skill in the art. Thus, numerical values described herein include variation of +/−0.1 to 10%, for example, +/−0.1%, 0.5%, 1%, 2%, 3%, 4%, 5%, 6%, 7%, 8%, 9%, or 10%. It is to be understood, although not always explicitly stated, that all numerical designations may be preceded by the term “about.” Thus, the term about includes variation of +/−0.1 to 10%, for example, +/−0.1%, 0.5%, 1%, 2%, 3%, 4%, 5%, 6%, 7%, 8%, 9%, or 10% of the numerical value. It is also to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein include the end points of the range, and include all values between the end points of the range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this disclosure.

As used herein, “immunotherapy” refers to the use of NK-92 cells, modified or unmodified, naturally occurring or modified NK cell or T-cell, whether alone or in combination, and which are capable of inducing cytotoxicity when contacting a target cell.

As used herein, “natural killer (NK) cells” are cells of the immune system that kill target cells in the absence of a specific antigenic stimulus, and without restriction according to major histocompatibility complex (MHC) class. Target cells may be tumor cells or cells harboring a virus. NK cells are characterized by the presence of CD56 and the absence of CD3 surface markers.

The term “endogenous NK cells” is used to refer to NK cells derived from a donor (or the patient), as distinguished from the NK-92 cell line. Endogenous NK cells are generally heterogeneous populations of cells within which NK cells have been enriched. Endogenous NK cells may be intended for autologous or allogeneic treatment of a patient.

The term “NK-92” refers to natural killer cells derived from the highly potent unique cell line described in Gong et al. (1994), rights to which are owned by NantKwest (hereafter, “NK-92™ cells”). The immortal NK cell line was originally obtained from a patient having non-Hodgkin's lymphoma. Unless indicated otherwise, the term “NK-92™” is intended to refer to the original NK-92 cell lines as well as NK-92 cell lines that have been modified (e.g., by introduction of exogenous genes). NK-92™ cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92™, NK-92T™-CD16, NK-92T™-CD16-y, NK-92T™-CD16-ζ NK-92T™-CD16(F 176V), NK-92™MI, and NK-92™CI. NK-92 cells are known to persons of ordinary skill in the art, to whom such cells are readily available from NantKwest, Inc.

The term “aNK” refers to an unmodified natural killer cells derived from the highly potent unique cell line described in Gong et al. (1994), rights to which are owned by NantKwest (hereafter, “aNK™ cells”). The term “haNK” refers to natural killer cells derived from the highly potent unique cell line described in Gong et al. (1994), rights to which are owned by NantKwest, modified to express CD16 on the cell surface (hereafter, “CD16+NK-92™ cells” or “haNK® cells”). In some embodiments, the CD16+ NK-92™ cells comprise a high affinity CD16 receptor on the cell surface. The term “taNK” refers to natural killer cells derived from the highly potent unique cell line described in Gong et al. (1994), rights to which are owned by NantKwest, modified to express a chimeric antigen receptor (hereafter, “CAR-modified NK-92™ cells” or “taNK® cells”). The term “t-haNK” refers to natural killer cells derived from the highly potent unique cell line described in Gong et al. (1994), rights to which are owned by NantkWest, modified to express CD 16 on the cell surface and to express a chimeric antigen receptor (hereafter, “CAR-modified CD16+NK-92™ cells” or “t-haNK™ cells”). In some embodiments, the t-haNK™ cells express a high affinity CD16 receptor on the cell surface.

A “modified NK-92 cell” refers to an NK-92 cell that expresses an exogenous gene or protein, such as an Fc receptor, a CAR, a cytokine (such as IL-2 or IL-12), and/or a suicide gene. In some embodiments, the modified NK-92 cell comprises a vector that encodes for a transgene, such as an Fc receptor, a CAR, a cytokine (such as IL-2 or IL-12), and/or a suicide gene. In one embodiment, the modified NK-92 cell expresses at least one transgenic protein.

As used herein, “non-irradiated NK-92 cells” are NK-92 cells that have not been irradiated. Irradiation renders the cells incapable of growth and proliferation. It is envisioned that the NK-92 cells will be irradiated at the treatment facility or some other point prior to treatment of a patient, since the time between irradiation and infusion should be no longer than four hours in order to preserve optimal activity. Alternatively, NK-92 cells may be prevented from proliferating by another mechanism.

As used herein, “inactivation” of the NK-92 cells renders them incapable of growth. Inactivation may also relate to the death of the NK-92 cells. It is envisioned that the NK-92 cells may be inactivated after they have effectively purged an ex vivo sample of cells related to a pathology in a therapeutic application, or after they have resided within the body of a mammal a sufficient period of time to effectively kill many or all target cells residing within the body. Inactivation may be induced, by way of non-limiting example, by administering an inactivating agent to which the NK-92 cells are sensitive.

As used herein, the terms “cytotoxic” and “cytolytic,” when used to describe the activity of effector cells such as NK-92 cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK-92 cells is due to cytolysis.

The term “kill” with respect to a cell/cell population is directed to include any type of manipulation that will lead to the death of that cell/cell population.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FCγR) bind to the IgG class of antibodies. FCγRIII-A (also called CD16; SEQ ID NO:20) is a low affinity Fc receptor bind to IgG antibodies and activate ADCC. FCγRIII-A are typically found on NK cells. NK-92 cells do not express FCγRIII-A. Fc-epsilon receptors (FccR) bind to the Fc region of IgE antibodies.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92 cell is targeted to cells expressing certain antigens on the cell surface, based on the specificity of the scFv domain. The scFv domain can be engineered to recognize any antigen, including tumor-specific antigens and virus-specific antigens. For example, CD19CAR recognizes CD19, a cell surface marker expressed by some cancers.

The term “tumor-specific antigen” as used herein refers to antigens that are present on a cancer or neoplastic cell but not detectable on a normal cell derived from the same tissue or lineage as the cancer cell. Tumor-specific antigens, as used herein, also refers to tumor-associated antigens, that is, antigens that are expressed at a higher level on a cancer cell as compared to a normal cell derived from the same tissue or lineage as the cancer cell.

The term “virus-specific antigen” as used herein refers to antigens that are present on a virus-infected cell but not detectable on a normal cell derived from the same tissue or lineage as the virus-infected cell. In one embodiment, a virus-specific antigen is a viral protein expressed on the surface of an infected cell.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, “percent identity” refers to sequence identity between two peptides or between two nucleic acid molecules. Percent identity can be determined by comparing a position in each sequence which may be aligned for purposes of comparison. When a position in the compared sequence is occupied by the same base or amino acid, then the molecules are identical at that position. Homologous nucleotide sequences include those sequences coding for naturally occurring allelic variants and mutations of the nucleotide sequences set forth herein. Homologous nucleotide sequences include nucleotide sequences encoding for a protein of a mammalian species other than humans. Homologous amino acid sequences include those amino acid sequences which contain conservative amino acid substitutions and which polypeptides have the same binding and/or activity. In some embodiments, a homologous amino acid sequence has no more than 15, nor more than 10, nor more than 5 or no more than 3 conservative amino acid substitutions. In some embodiments, a nucleotide or amino acid sequence has at least 60%, at least 65%, at least 70%, at least 80%, or at least 85% or greater percent identity to a sequence described herein. In some embodiments, a nucleotide or amino acid sequence has at least 90%, 91%, 92%, 93%, 94%, 95%, 96%, 97%, 98% or 99% identity to a sequence described herein. Percent identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). Algorithms suitable for determining percent sequence identity include the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (see the internet at ncbi.nlm.nih.gov). The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4.

In some embodiments, a nucleic acid sequence is codon optimized for expression in a particular species, for example, a mouse sequence can be codon optimized for expression in humans (expression of the protein encoded by the codon-optimized nucleic acid sequence). Thus, in some embodiments, a codon-optimized nucleic acid sequence has at least 60%, at least 65%, at least 70%, at least 80%, or at least 85% or greater percent identity to a nucleic acid sequence described herein. In some embodiments, a codon-optimized nucleic acid sequence acid sequence has at least 90%, 91%, 92%, 93%, 94%, 95%, 96%, 97%, 98% or 99% identity to a sequence described herein.

The term “express” refers to the production of a gene product (e.g., a protein). The term “transient” when referring to expression means a polynucleotide is not incorporated into the genome of the cell. The term “stable” when referring to expression means a polynucleotide is incorporated into the genome of the cell, or a positive selection marker (i.e., an exogenous gene expressed by the cell that confers a benefit under certain growth conditions) is utilized to maintain expression of the transgene.

The term “cytokine” or “cytokines” refers to the general class of biological molecules which affect cells of the immune system. Exemplary cytokines include but are not limited to interferons and interleukins (IL) —in particular—IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited or no ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA. In one embodiment, the vector is a viral vector, e.g. adenovirus. Viral vectors are well known in the art.

As used herein, the term “targeted,” when referring to protein expression, is intended to include, but is not limited to, directing proteins or polypeptides to appropriate destinations in the cell or outside of it. The targeting is typically achieved through signal peptides or targeting peptides, which are a stretch of amino acid residues in a polypeptide chain. These signal peptides can be located anywhere within a polypeptide sequence, but are often located on the N-terminus. Polypeptides can also be engineered to have a signal peptide on the C-terminus. Signal peptides can direct a polypeptide for extracellular section, location to plasma membrane, golgi, endosomes, endoplasmic reticulum, and other cellular compartments. For example, polypeptides with a particular amino acid sequence on their C-terminus (e.g., KDEL) are retained in the ER lumen or transported back the ER lumen.

As used herein, the term “target,” when referring to targeting of a tumor, refers to the ability of NK-92 cells to recognize and kill a tumor cell (i.e., target cell). The term “targeted” in this context refers, for example, to the ability of a CAR expressed by the NK-92 cell to recognize and bind to a cell surface antigen expressed by the tumor.

As used herein, the term “transfect” refers to the insertion of nucleic acid into a cell. Transfection may be performed using any means that allows the nucleic acid to enter the cell. DNA and/or mRNA may be transfected into a cell. Preferably, a transfected cell expresses the gene product (i.e., protein) encoded by the nucleic acid.

The term “suicide gene” refers to a transgene that allows for the negative selection of cells expressing that transgene. A suicide gene is used as a safety system, allowing the cells expressing the gene to be killed by introduction of a selective agent. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene (see also, for example, Yazawa K, Fisher W E, Brunicardi F C: Current progress in suicide gene therapy for cancer. World J. Surg. 2002 July; 26(7):783-9). In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

What is claimed is:

1. A genetically modified NK cell carrying a non-viral vector encoding a membrane bound recombinant chimeric antigen receptor (CAR),
wherein the CAR comprises in a single polypeptide chain:
an extracellular scFv domain comprise a sequence selected from the group consisting of SEQ ID NOs: 26, 28, 30, 32, 34, 36, 40, 42, 49, 51, 53, 58, and 59,
a hinge domain,
a transmembrane domain, and
a FcϵRIγ signaling domain according to SEQ ID NO:1; and
wherein the NK cell is an NK-92 cell.

2. The genetically modified NK cell of claim 1, wherein the hinge domain comprises a CD8 hinge domain according to SEQ ID NO:6 or SEQ ID NO:45, and/or wherein the transmembrane domain comprises a CD28 transmembrane domain according to SEQ ID NO:7.

3. The genetically modified NK cell of claim 1, further carrying a membrane bound recombinant CD16.

4. The genetically modified NK cell of claim 1, further comprising a recombinant cytokine with an endoplasmic retention sequence.

5. The genetically modified NK cell of claim 1, wherein the CAR comprises in a single polypeptide chain: an extracellular scFv domain comprising a sequence selected from the group consisting of SEQ ID NOs: 26, 28, 30, 32, 34, 36, 40, 42, 49, 51, 53, 58, and 59, a hinge domain according to SEQ ID NO:6 or SEQ ID NO:45, a transmembrane domain according to SEQ ID NO:7, and a FcϵRIγ signaling domain according to SEQ ID NO:1.

6. A genetically modified NK cell, comprising:
a recombinant nucleic acid encoding a chimeric antigen receptor (CAR), wherein the recombinant nucleic acid is a transfected plasmid;
wherein the NK cell is an NK-92 cell;
wherein the CAR comprises in a single polypeptide chain an extracellular binding domain comprising a sequence selected from the group consisting of SEQ ID NOs: 26, 28, 30, 32, 34, 36, 40, 42, 49, 51, 53, 58, and 59, a hinge domain, a transmembrane domain, and a FcϵRIγ signaling domain according to SEQ ID NO:1.

7. The genetically modified NK cell of claim 6, wherein the recombinant nucleic acid is an RNA.

8. The genetically modified NK cell of claim 7, wherein the RNA is a polycistronic RNA that further encodes a CD16 and/or a cytokine with an endoplasmic retention sequence.

9. The genetically modified NK cell of claim 6, wherein the extracellular binding domain comprises a scFv.

10. The genetically modified NK cell of claim 6, wherein the extracellular binding domain specifically binds to a tumor-specific antigen, a tumor associated antigen, or a patient- and tumor-specific antigen.

11. The genetically modified NK cell of claim 10, wherein the tumor-specific antigen is CD20, GD2, CD30, EGFR, FAP, CD33, CD123, PD-L1, IGF1R, CSPG4, or B7-H4.

12. The genetically modified NK cell of claim 6, wherein the extracellular binding domain specifically binds to a virus-specific antigen.

13. The genetically modified NK cell of claim 12, wherein the virus-specific antigen is an antigen of a HIV virus, an HPV virus, an RSV virus, an influenza virus, an ebolavirus, or an HCV virus.

14. The genetically modified NK cell of claim 12, wherein the virus-specific antigen is gp120 of an HIV virus.

15. The genetically modified NK cell of claim 6, wherein the hinge domain and/or the transmembrane domain comprise a CD8 hinge domain and/or a CD28 transmembrane domain.

16. A method of treating cancer in a patient in need thereof, comprising administering to the patient a therapeutically effective amount of the genetically modified NK cells of claim 1, thereby treating the cancer.

17. The method of claim 16, further comprising a step of administering at least one additional therapeutic entity selected from the group consisting of a viral cancer vaccine, a bacterial cancer vaccine, a yeast cancer vaccine, N-803, an antibody, a stem cell transplant, and a tumor targeted cytokine.

18. The method of claim 16, wherein the cancer is selected from leukemia, acute lymphocytic leukemia, acute myelocytic leukemia, chronic leukemias, chronic myelocytic (granulocytic) leukemia, chronic lymphocytic leukemia, polycythemia vera, lymphomas, Hodgkin's disease, non-Hodgkin's disease, multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

19. A method of treating a viral infection in a patient in need thereof, comprising administering to the patient a therapeutically effective amount of the genetically modified NK cells of claim 1, thereby treating the viral infection.

20. The method of claim 19, further comprising administering an antiviral drug.


SEQUENCE LISTING (76 sequences)

SEQ ID NO: 1 - protein, 41 aa, synthetic construct
  REGION 1..41: note = FceRIgamma Intracellular Domain peptide
LKIQVRKAAITSYEKSDGVYTGLSTRNQETYETLKHEKPPQ

SEQ ID NO: 2 - DNA, 123 bp, synthetic construct
  misc_feature 1..123: note = FceRIgamma Intracellular Domain
  source 1..123: mol_type = other DNA
ctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 3 - protein, 398 aa, synthetic construct
  REGION 1..398: note = CD19CAR FceRIgamma
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSAAALFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 4 - protein, 259 aa, synthetic construct
  REGION 1..259: note = CD19CAR scFv
AATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 5 - DNA, 1197 bp, synthetic construct
  misc_feature 1..1197: note = CD19CAR FceRIgamma DNA
  source 1..1197: mol_type = other DNA
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccagtaa

SEQ ID NO: 6 - protein, 54 aa, synthetic construct
  REGION 1..54: note = CD8 Hinge
KPTTTPAPRPPTPAPTIASQPLSLRPEACRPAAGGAVHTRGLDFACFWVLVVVG

SEQ ID NO: 7 - protein, 20 aa, synthetic construct
  REGION 1..20: note = CD28 Transmembrane domain
GVLACYSLLVTVAFIIFWVR

SEQ ID NO: 8 - protein, 41 aa, synthetic construct
  REGION 1..41: note = CD28 signaling domain
RSKRSRLLHSDYMNMTPRRPGPTRKHYQPYAPPRDFAAYRS

SEQ ID NO: 9 - protein, 42 aa, synthetic construct
  REGION 1..42: note = 4-1BB signaling domain
KRGRKKLLYIFKQPFMRPVQTTQEEDGCSCRFPEEEEGGCEL

SEQ ID NO: 10 - protein, 113 aa, synthetic construct
  REGION 1..113: note = CD3 zeta signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - DNA, 339 bp, synthetic construct
  misc_feature 1..339: note = Human CD3 zeta (codon optimized):
  source 1..339: mol_type = other DNA
gtgaagtttagcagatctgccgacgcccctgcctaccagcagggacagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 12 - DNA, 339 bp, synthetic construct
  misc_feature 1..339: note = Human CD3 zeta (non-codon optimized)
  source 1..339: mol_type = other DNA
gtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 13 - DNA, 1410 bp, synthetic construct
  misc_feature 1..1410: note = CD19CAR CD3z (DNA sequence)
  source 1..1410: mol_type = other DNA
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcaaggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgagtgaagttcagcagatccgccgatgcccctgcttaccagcagggccagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 14 - DNA, 1533 bp, synthetic construct
  misc_feature 1..1533: note = CD19CAR CD28/CD3z (DNA sequence)
  source 1..1533: mol_type = other DNA
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgaagcaagcggagcaggctgctgcacagcgactacatgaacatgacccctagaaggcctggccccaccagaaagcactatcagccttacgcccctcccagagacttcgccgcctacagatccagagtgaagttcagcagatctgccgacgcccctgcttaccagcagggccagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 15 - DNA, 1536 bp, synthetic construct
  misc_feature 1..1536: note = CD19CAR_4-1BB/CD3z (DNA sequence)
  source 1..1536: mol_type = other DNA
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtcaagcggggcagaaagaagctgctgtacatcttcaagcagcccttcatgaggcccgtgcagaccacacaggaagaggacggctgcagctgtagattccctgaggaagaagaaggcggctgcgagctgagagtgaagtttagcagatctgccgacgcccctgcctaccagcagggacagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 16 - DNA, 1659 bp, synthetic construct
  misc_feature 1..1659: note = CD19CAR_CD28/4-1BB/CD3z (DNA sequence)
  source 1..1659: mol_type = other DNA
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgaagcaagcggagcaggctgctgcacagcgactacatgaacatgacccctagaaggcctggccccaccagaaagcactatcagccttacgcccctcccagagacttcgccgcctatagatccaagcggggcagaaagaagctgctgtacatcttcaagcagcccttcatgaggcccgtgcagaccacacaggaagaggacggctgcagctgtagattccctgaggaagaagaaggcggctgcgagctgagagtgaagtttagcagatctgccgacgcccctgcctaccagcagggacagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 17 - DNA, 1659 bp, synthetic construct
  misc_feature 1..1659: note = CD19CAR_4-1BB/CD3z/CD28 (DNA sequence)
  source 1..1659: mol_type = other DNA
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtcaagcggggcagaaagaagctgctgtacatcttcaagcagcccttcatgaggcccgtgcagaccacacaggaagaggacggctgcagctgtagattccctgaggaagaagaaggcggctgcgagctgagagtgaagtttagcagatctgccgacgcccctgcctaccagcagggacagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagaagaagcaagagatctagactgctgcacagcgactacatgaacatgacccctagaaggcctggccccaccagaaagcactatcagccttacgcccctcccagagacttcgccgcctacagatcttga

SEQ ID NO: 18 - protein, 153 aa, synthetic construct
  REGION 1..153: note = Wild-Type IL-2 (amino acid sequence)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 19 - protein, 160 aa, synthetic construct
  REGION 1..160: note = IL-2-ER (amino acid sequence)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 20 - DNA, 765 bp, synthetic construct
  misc_feature 1..765: note = Polynucleotide Encoding the Low Affinity Immunoglobulin Gamma FcRegion Receptor III-A
  source 1..765: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 21 - DNA, 1455 bp, synthetic construct
  misc_feature 1..1455: note = CD19-CAR DNA sequence (murine)
  source 1..1455: mol_type = other DNA
cccgggaattcgccaccatggactggatctggcggatcctgttcctcgtgggagccgccacaggcgcccattctgcccagcccgccgacatccagatgacccagaccaccagcagcctgagcgccagcctgggcgacagagtgaccatcagctgccgggccagccaggacatcagcaagtacctgaactggtatcagcagaaacccgacggcaccgtgaagctgctgatctaccacaccagccggctgcacagcggcgtgcccagcagattttctggcagcggcagcggcaccgactacagcctgaccatctccaacctggaacaggaagatatcgctacctacttctgtcagcaaggcaacaccctgccctacaccttcggcggaggcaccaagctggaactgaagagaggcggcggaggctctggtggaggcggatctgggggcggaggaagtggcgggggaggatctgaagtgcagctgcagcagagcggccctggcctggtggcccctagccagagcctgtccgtgacctgtaccgtgtccggcgtgtccctgcccgactacggcgtgtcctggatccggcagccccccagaaagggcctggaatggctgggcgtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgtccagcgccctgtccaacagcatcatgtacttcagccacttcgtgcccgtgtttctgcccgccaagcccaccaccacccctgcccctagacctcccaccccagccccaacaatcgccagccagcctctgtccctgcggcccgaagctagcagacctgctgccggcggagccgtgcacaccagaggcctggaccccaagctgtgctacctgctggacggcatcctgttcatctatggcgtgatcctgaccgccctgttcctgagagtgaagttcagcagaagcgccgacgcccctgcctaccagcagggccagaaccagctgtacaacgagctgaacctgggcagacgggaagagtacgacgtgctggacaagcggagaggcagggaccccgagatgggcggcaagcccagacggaagaacccccaggaaggcctgtataacgaactgcagaaagacaagatggccgaggcctacagcgagatcggcatgaagggcgagcggcggaggggcaagggccacgatggactgtaccagggcctgagcaccgccaccaaggacacctacgacgccctgcacatgcaggccctgccccccagatgacagccagggcatttctccctcgagcggccgc

SEQ ID NO: 22 - protein, 468 aa, synthetic construct
  REGION 1..468: note = CD19-CAR amino acids sequence (murine)
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSALSNSIMYFSHFVPVFLPAKPTTTPAPRPPTPAPTIASQPLSLRPEASRPAAGGAVHTRGLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 813 bp, synthetic construct
  misc_feature 1..813: note = Codon-optimized CD19 scFv - DNA sequence
  source 1..813: mol_type = other DNA
atggactggatctggcggattctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatct

SEQ ID NO: 24 - protein, 271 aa, synthetic construct
  REGION 1..271: note = CD19 scFv - Protein sequence
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 25 - DNA, 807 bp, synthetic construct
  misc_feature 1..807: note = Codon-optimized CD20 scFv - DNA sequence
  source 1..807: mol_type = other DNA
atggactggatctggcgcatcctcttcctcgtcggcgctgctaccggcgctcattcggcccagccggccatggcgcaagtaaaactccaagaatctggggcggagctggtgaaaccgggggcgtctgtgaagatgagctgtaaagcatcaggctacaccttcacctcctataatatgcactgggtgaaacaaacacccggacagggcctcgaatggattggtgccatctatcctggaaatggtgatacctcatataatcagaagtttaagggcaaggctacgcttactgcggataaaagctcttccactgcttacatgcaactgagcagtctcacttcagaggactcagccgattattattgtgcccgcagcaactactatggtagttcatactggtttttcgacgtttgggggcaaggtaccaccgtcacggtttcttctggtgggggcggaagcgggggtggaggatctgggggcggtggttcagacattgaactcacccagagccctactattctgagcgcgtctccaggtgaaaaagttacgatgacgtgcagagcatcaagtagtgtgaattatatggattggtatcaaaagaagccaggctcatccccaaaaccgtggatctatgcaactagcaacctcgcgtcaggggtgccagcaaggttttccggaagtggttctggcacatcttatagtctcaccatttcccgagtggaggctgaggatgcggccacttattactgccagcaatggtcattcaatcccccaacatttggtggcggaacaaaactcgaaattaaacgg

SEQ ID NO: 26 - protein, 269 aa, synthetic construct
  REGION 1..269: note = CD20 scFv - Protein sequence
MDWIWRILFLVGAATGAHSAQPAMAQVKLQESGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGQGTTVTVSSGGGGSGGGGSGGGGSDIELTQSPTILSASPGEKVTMTCRASSSVNYMDWYQKKPGSSPKPWIYATSNLASGVPARFSGSGSGTSYSLTISRVEAEDAATYYCQQWSFNPPTFGGGTKLEIKR

SEQ ID NO: 27 - DNA, 795 bp, synthetic construct
  misc_feature 1..795: note = Codon-optimized CD33 scfV - DNA sequence:
  source 1..795: mol_type = other DNA
atggactggatctggcgcatcctcttcctcgtcggcgctgctaccggcgctcattcggcccagccggccgacattcaaatgactcagtccccttccagcttgtcagcctcagtaggggaccgggtcacgatcacctgtcgagcgtctgagtcagtggataactacgggatttctttcatgaactggttccagcagaagcccggcaaagctcctaagctccttatatatgcagcctcaaatcaggggagcggtgttcctagtcgcttcagtggaagcggtagcggtacggactttacgttgacgataagtagccttcagccagatgactttgccacttattattgtcagcagtctaaggaagttccttggacgtttggccaaggaacgaaggtcgaaatcaaagggggagggggctcaggagggggcggcagtggtggtggaggctctcaagtccaactcgtacagtctggcgcggaggttaaaaagccgggaagctccgtgaaagtatcctgtaaggcaagcggatacacctttaccgattataacatgcactgggttaggcaggcgcccggccaaggtctggaatggatcggttatatttatccatacaacggtggtaccggctataatcagaagtttaagagtaaggctactattacagcggatgagtcaaccaatactgcatacatggagctctcctcactcaggagcgaagataccgcagtgtattactgtgcccgagggagaccagccatggactactggggtcagggtacccttgtgacagtatctagc

SEQ ID NO: 28 - protein, 265 aa, synthetic construct
  REGION 1..265: note = CD33 scfV - Protein sequence
MDWIWRILFLVGAATGAHSAQPADIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 29 - DNA, 840 bp, synthetic construct
  misc_feature 1..840: note = Codon-optimized CSPG4 scfV - DNA sequence
  source 1..840: mol_type = other DNA
atggactggatctggcgcatcctcttcctcgtcggcgctgctaccggcgctcattcggcccagccggccgatatcgagctcacccaatctccaaaattcatgtccacatcagtaggagacagggtcagcgtcacctgcaaggccagtcagaatgtggatactaatgtagcgtggtatcaacaaaaaccagggcaatctcctgaaccactgcttttctcggcatcctaccgttacactggagtccctgatcgcttcacaggcagtggatctgggacagatttcactctcaccatcagcaatgtgcagtctgaagacttggcagagtatttctgtcagcaatataacagctatcctctgacgttcggtggcggcaccaagctggaaatcaaacgggctgccgcagaaggtggaggcggttcaggtggcggaggttccggcggaggtggctctggcggtggcggatcggccatggcccaggtgaagctgcagcagtcaggagggggcttggtgcaacctggaggctccatgaaactctcctgtgttgtctctggattcactttcagtaattactggatgaactgggtccgccagtctccagagaaggggcttgagtggattgcagaaattagattgaaatccaataattttggaagatattatgcggagtctgtgaaagggaggttcaccatctcaagagatgattccaaaagtagtgcctacctgcaaatgatcaacctaagagctgaagatactggcatttattactgtaccagttatggtaactacgttgggcactattttgaccactggggccaagggaccacggtcaccgtatcgagt

SEQ ID NO: 30 - protein, 280 aa, synthetic construct
  REGION 1..280: note = CSPG4 scfV - Protein sequence
MDWIWRILFLVGAATGAHSAQPADIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRAAAEGGGGSGGGGSGGGGSGGGGSAMAQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSS

SEQ ID NO: 31 - DNA, 807 bp, synthetic construct
  misc_feature 1..807: note = Codon-optimized EGFR scFv - DNA sequence
  source 1..807: mol_type = other DNA
atggactggatctggcggattctgtttctcgtgggagctgccacaggcgctcattctgctcagcctgccgatattcttcttactcaatctcccgttattttgtcagtatccccaggtgagcgagtcagcttctcttgtcgagcgtcacaatccattggcaccaacatacattggtaccaacagcgcaccaacgggtctccccggctcttgattaagtacgcatcagaaagtatttctgggatacccagtaggttctcagggagcgggagtggcactgactttaccctgtccataaacagcgttgagtctgaggacatcgcggactactattgtcagcagaacaacaattggccgaccacgtttggtgcgggaacaaaacttgaactcaaaggcggcggaggaagcggaggcggaggatctgggggcggaggctctggcggagggggatctcaggtgcagctcaaacagtcaggacctggcctcgttcagccaagccaatcactgagtataacgtgcacggtgagcggctttagcctgacaaactatggtgtccactgggtccgccaatctcctggaaaaggcttggagtggctcggtgttatctggtccggtggtaacacagactacaacacgccattcaccagtcgccttagtattaacaaggacaactccaagtctcaggttttctttaaaatgaactctctgcagtctaatgataccgcaatttactactgtgcgagggcactcacgtactatgactatgagttcgcgtattggggccaagggactctcgttactgtctcagcg

SEQ ID NO: 32 - protein, 269 aa, synthetic construct
  REGION 1..269: note = EGFR scFv - Protein sequence
MDWIWRILFLVGAATGAHSAQPADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 33 - DNA, 822 bp, synthetic construct
  misc_feature 1..822: note = Codon-optimized IGF1R scFv - DNA sequence
  source 1..822: mol_type = other DNA
atggactggatctggcggattctgtttctcgtgggagctgccacaggcgctcattctgctcagcctgccgatgttgtaatgacgcagtcacccctgtcactcccggtcacacccggagaaccagcgtcaattagctgccgatctagccaaagtttgcttcattccaatggttacaattatctcgactggtacttgcagaaacccggccaatcccctcagctgctcatctaccttgggtctaatagggcatctggggttcccgataggttctctggctccgggagcggcaccgactttacgttgaaaatctctagggttgaggcggaagacgtaggcgtttactattgcatgcaggggacccactggccgctgaccttcggccagggcaccaaggttgaaataaaaggcggcggaggaagcggaggcggaggatctgggggcggaggctctggcggagggggatctcaggtacagctccaggaatcaggacccggtttggttaagccctccgggaccctttccctcacgtgtgcagtctcaggtgggtcaattagttcttccaattggtggtcttgggtgcggcaaccacctggtaaaggtctcgagtggataggggaaatttatcatagtggctccaccaattataacccctcactcaagtccagggttacgatatctgtggacaaaagtaaaaaccaattctccctcaaacttagtagtgtaacagcggcagacaccgcggtgtactactgcgcacggtggacaggccgaactgatgcctttgacatttggggacagggaactatggtgactgtgtcatcc

SEQ ID NO: 34 - protein, 274 aa, synthetic construct
  REGION 1..274: note = IGF1R scFv -Protein sequence
MDWIWRILFLVGAATGAHSAQPADVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKGGGGSGGGGSGGGGSGGGGSQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSS

SEQ ID NO: 35 - DNA, 822 bp, synthetic construct
  misc_feature 1..822: note = Codon-optimized CD30 scFv - DNA sequence
  source 1..822: mol_type = other DNA
atggactggatctggcggattctgtttctcgtgggagctgccacaggcgctcattctgctcagcctgccgatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgtaagtaacttggaaactggcgtcccaagccgcttctctggatctggttcaggcaccgacttcactttcactatcagcagcctgcagcctgaagatatcgcaacctactattgccagcaggttgctaatgttcctctgactttcggccaaggcaccaaggtggagatcaagggcggcggaggaagcggaggcggaggatctgggggcggaggctctggcggagggggatctgaagttcagcttgtagaatctggaggtggattggttcaacctggtggctctcttcgcctgagttgtgcagcctctggttttactttctctagttactggatgcattgggttcgtcaggctcctgggaaaggcctggaatgggtttcagctattagttggagtggagatagtacttactacgcagacagtgtgaaaggtcgcttcaccatcagccgtgataattctaagaacactttgtacctgcaaatgaactccttgcgcgcagaagacacggctgtgtactattgtgcccgtgatcgctctgcgacttggtattatctggggcttggtttcgatgtatggggacaaggtaccctggtaacggtttctagc

SEQ ID NO: 36 - protein, 274 aa, synthetic construct
  REGION 1..274: note = CD30 scFv - Protein sequence
MDWIWRILFLVGAATGAHSAQPADIQMTQSPSSLSASVGDRVTITCQASQDISNYLNWYQQKPGKAPKLLIYDVSNLETGVPSRFSGSGSGTDFTFTISSLQPEDIATYYCQQVANVPLTFGQGTKVEIKGGGGSGGGGSGGGGSGGGGSEVQLVESGGGLVQPGGSLRLSCAASGFTFSSYWMHWVRQAPGKGLEWVSAISWSGDSTYYADSVKGRFTISRDNSKNTLYLQMNSLRAEDTAVYYCARDRSATWYYLGLGFDVWGQGTLVTVSS

SEQ ID NO: 37 - DNA, 825 bp, synthetic construct
  misc_feature 1..825: note = Codon-optimized HER2/neu scFv - DNA sequence
  source 1..825: mol_type = other DNA
atggactggatctggcggattctgtttctcgtgggagctgccacaggcgctcattctgctcagcctgccgacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagtcctcagggggcgggggaagtggtgggggcggcagcggcggagggggctcaggaggaggcggatcaggcggatcagaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagc

SEQ ID NO: 38 - protein, 275 aa, synthetic construct
  REGION 1..275: note = HER2/neu scFv - Protein sequence
MDWIWRILFLVGAATGAHSAQPADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSSGGGGSGGGGSGGGGSGGGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 39 - DNA, 798 bp, synthetic construct
  misc_feature 1..798: note = Codon-optimized GD2 scFv - DNA sequence VL/VH
  source 1..798: mol_type = other DNA
atggactggatctggcggattctgtttctcgtgggagctgccacaggcgctcattctgctcagcctgccagcatcgtgatgacccagactcctaagttcctgctggtgtctgccggcgacagagtgaccatcacctgtaaagccagccagagcgtgtccaacgacgtggcctggtatcagcagaagcctggacagagccccaagctgctgatctacagcgccagcaacagatacaccggcgtgcccgatagattcaccggctctggctacggcaccgacttcacctttaccatcagcaccgtgcaggccgaggatctggccgtgtacttctgccagcaagactacagctctctcggcggaggcaccaagctggaaatcaaaggcggcggaggaagcggaggcggaggatctgggggcggaggctctggcggagggggatctcaggtgcaagtgaaagagtctggccctggactggtggccccaagccagtctctgagcatcacatgtaccgtgtccggcttcagcctgaccaactatggcgtgcactgggtccgacagcctccaggcaaaggactggaatggctgggagtgatttgggctggcggcagcaccaactacaacagcgccctgatgagccggctgagcatctccaaggacaacagcaagagccaggtgttcctgaagatgaacagcctgcagaccgacgacaccgccatgtactactgtgctagcagaggcggcaactacggctacgccctggattattggggccagggcacaagcgtgaccgtgtcatct

SEQ ID NO: 40 - DNA, 798 bp, synthetic construct
  misc_feature 1..798: note = Codon-optimized GD2 scFv - DNA sequence VH/VL
  source 1..798: mol_type = other DNA
atggactggatctggcggattctgtttctcgtgggagctgccacaggcgctcattctgctcagcctgcccaggtgcaagtgaaagagtctggccctggactggtggccccaagccagtctctgagcatcacatgtaccgtgtccggcttcagcctgaccaactatggcgtgcactgggtccgacagcctccaggcaaaggactggaatggctgggagtgatttgggctggcggcagcaccaactacaacagcgccctgatgagccggctgagcatctccaaggacaacagcaagagccaggtgttcctgaagatgaacagcctgcagaccgacgacaccgccatgtactactgtgctagcagaggcggcaactacggctacgccctggattattggggccagggcacaagcgtgaccgtgtcatctggcggcggaggaagcggaggcggaggatctgggggcggaggctctggcggagggggatctagcatcgtgatgacccagactcctaagttcctgctggtgtctgccggcgacagagtgaccatcacctgtaaagccagccagagcgtgtccaacgacgtggcctggtatcagcagaagcctggacagagccccaagctgctgatctacagcgccagcaacagatacaccggcgtgcccgatagattcaccggctctggctacggcaccgacttcacctttaccatcagcaccgtgcaggccgaggatctggccgtgtacttctgccagcaagactacagctctctcggcggaggcaccaagctggaaatcaaa

SEQ ID NO: 41 - protein, 266 aa, synthetic construct
  REGION 1..266: note = GD2 scFv - Protein sequence VL/VH
MDWIWRILFLVGAATGAHSAQPASIVMTQTPKFLLVSAGDRVTITCKASQSVSNDVAWYQQKPGQSPKLLIYSASNRYTGVPDRFTGSGYGTDFTFTISTVQAEDLAVYFCQQDYSSLGGGTKLEIKGGGGSGGGGSGGGGSGGGGSQVQVKESGPGLVAPSQSLSITCTVSGFSLTNYGVHWVRQPPGKGLEWLGVIWAGGSTNYNSALMSRLSISKDNSKSQVFLKMNSLQTDDTAMYYCASRGGNYGYALDYWGQGTSVTVSS

SEQ ID NO: 42 - protein, 266 aa, synthetic construct
  REGION 1..266: note = GD2 scFv - Protein sequence VH/VL
MDWIWRILFLVGAATGAHSAQPAQVQVKESGPGLVAPSQSLSITCTVSGFSLTNYGVHWVRQPPGKGLEWLGVIWAGGSTNYNSALMSRLSISKDNSKSQVFLKMNSLQTDDTAMYYCASRGGNYGYALDYWGQGTSVTVSSGGGGSGGGGSGGGGSGGGGSSIVMTQTPKFLLVSAGDRVTITCKASQSVSNDVAWYQQKPGQSPKLLIYSASNRYTGVPDRFTGSGYGTDFTFTISTVQAEDLAVYFCQQDYSSLGGGTKLEIK

SEQ ID NO: 43 - protein, 254 aa, synthetic construct
  REGION 1..254: note = High Affinity Variant Ig gamma FcRIII-A amino acid sequence
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 44 - DNA, 765 bp, synthetic construct
  misc_feature 1..765: note = High Affinity Variant IgGamma FcRIII-A nucleic acid sequence
  source 1..765: mol_type = other DNA
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 45 - protein, 61 aa, synthetic construct
  REGION 1..61: note = CD8 Hinge peptide (Human)
LSNSIMYFSHFVPVFLPAKPTTTPAPRPPTPAPTIASQPLSLRPEACRPAAGGAVHTRGLD

SEQ ID NO: 46 - DNA, 183 bp, synthetic construct
  misc_feature 1..183: note = CD8 Hinge DNA (Human)
  source 1..183: mol_type = other DNA
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggat

SEQ ID NO: 47 - protein, 135 aa, synthetic construct
  REGION 1..135: note = Human T-cell surface glycoprotein CD3 zeta
PKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 822 bp, synthetic construct
  misc_feature 1..822: note = Codon-optimized CD123 scFv DNA
  source 1..822: mol_type = other DNA
atggactggatctggcggattctgtttctcgtgggagctgccacaggcgctcattctgctcagcctgccctgcccgtcctgacacagtccgcaagtgtgagtggatcacctggacaatcaattactatcagttgcacaggcacttcctcagacgtagggaggtatgattacgtgtcatggtatcaacagcatccaggcaaagctcctcagctcatgatttatgatgtgtccaacagaccgtccggagtatctaatcgcttcagtggatctaaatccggtaatactgcctccctcaccatatcagggctccaggccgaagatgaagcagactactattgcagtagttacactggttcaagtacgctttacgtttttggcacggggaccaaggtaacggtcctgggccaacccaaaggcggaggagggtccggtggcggtggcagtggtggagggggatcagaggtgcaattggttgagagcggtggtgggctggttaaacctggcgggtccctccgcttgtcttgtgccgcaagcgggtttacctttagtaatgcgtggatgagctgggtgcgacaagcacccggaaagggcctggagtgggtcggtaggattaaaagcaaaacagatggtggaacaaccgattatgcggccccagtcaagggaaggttcactatttcaagagacgattccaagaacactctttacctccaaatgaatagtttgaaaacagaggatacagcagtgtactattgcacaacggactacgacttttggagcggatattactactgggggcaaggtaccctggtcacagtttcatca

SEQ ID NO: 49 - protein, 274 aa, synthetic construct
  REGION 1..274: note = CD123 scFv Protein sequence
MDWIWRILFLVGAATGAHSAQPALPVLTQSASVSGSPGQSITISCTGTSSDVGRYDYVSWYQQHPGKAPQLMIYDVSNRPSGVSNRFSGSKSGNTASLTISGLQAEDEADYYCSSYTGSSTLYVFGTGTKVTVLGQPKGGGGSGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDYDFWSGYYYWGQGTLVTVSS

SEQ ID NO: 50 - DNA, 807 bp, synthetic construct
  misc_feature 1..807: note = Codon-optimized PD-L1 scFvDNA
  source 1..807: mol_type = other DNA
atggactggatctggcggattctgtttctcgtgggagctgccacaggcgctcattctgctcagcctgccaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctca

SEQ ID NO: 51 - protein, 269 aa, synthetic construct
  REGION 1..269: note = PD-L1 scFv Protein
MDWIWRILFLVGAATGAHSAQPANIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKGGGGSGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSS

SEQ ID NO: 52 - DNA, 807 bp, synthetic construct
  misc_feature 1..807: note = Codon-optimized B7-H4 scFvDNA
  source 1..807: mol_type = other DNA
atggactggatctggcggattctgtttctcgtgggagctgccacaggcgctcattctgctcagcctgccgaagttcagcttgtagaatctggaggtggattggttcaacctggtggctctcttcgcctgagttgtgcagcctctggttttactttcaatagttacgctatgcattgggttcgtcaggctcctgggaaaggcctggaatgggtttcagctattagtggtaatggaggtagtactcgttacgcagacagtgtgaaaggtcgcttcaccatcagccgtgataattctaagaacactttgtacctgcaaatgaactccttgcgcgcagaagacacggctgtgtactattgtgcccgtgatcgctttcggaaggttcatggtttcgatgtatggggacaaggtaccctggtaacggtttctagcggaggtggtgggagtggtggaggcggctcgggtggaggtggttcaggaggaggcggagatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgcaagtaacttggaaactggcgtcccaagccgcttctctggatctggttcaggcaccgacttcactttcactatcagcagcctgcagcctgaagatatcgcaacctactattgccagcaggatgctacttttcctttgactttcggccaaggcaccaaggtggagatcaag

SEQ ID NO: 53 - protein, 269 aa, synthetic construct
  REGION 1..269: note = B7-H4 scFvProtein
MDWIWRILFLVGAATGAHSAQPAEVQLVESGGGLVQPGGSLRLSCAASGFTFNSYAMHWVRQAPGKGLEWVSAISGNGGSTRYADSVKGRFTISRDNSKNTLYLQMNSLRAEDTAVYYCARDRFRKVHGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQDATFPLTFGQGTKVEIK

SEQ ID NO: 54 - DNA, 1188 bp, synthetic construct
  misc_feature 1..1188: note = Codon-optimized HIV-gp120 binding domain (CD4)DNA
  source 1..1188: mol_type = other DNA
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagcca

SEQ ID NO: 55 - protein, 396 aa, synthetic construct
  REGION 1..396: note = HIV-gp120 binding domain (CD4) Protein
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQP

SEQ ID NO: 56 - DNA, 840 bp, synthetic construct
  misc_feature 1..840: note = Codon-optimized FAP scFv VL/VH DNA
  source 1..840: mol_type = other DNA
atggactggatctggcggattctgtttctcgtgggagctgccacaggcgctcattctgctcagcctgccgacatcgtgatgacacagagcccttctagcctggccgtgtccgtgggagagaaagtgaccatgagctgcaagagcagccagagcctgctgtactcccggaaccagaagaactacctggcctggttccagcagaagcccggccagtctcctaagctgctgatcttctgggccagcaccagagaaagcggcgtgcccgatagattcaccggcagcggctttggcaccgacttcaacctgacaatcagcagcgtgcaggccgaggacctggctgtgtacgattgccagcagtacttcagctaccctctgacctttggagccggcaccaagctggaactgagaggcggcggaggaagcggaggcggaggatctgggggcggaggctctggcggagggggatctcaggttcagctgcagcagtctggacctgagctggttaagcctggcgcctccgtgaagatgagctgcaagaccagccggtacaccttcaccgagtacaccatccactgggtccgacagagccacggcaagagcctggaatggatcggcggcatcaaccccaacaacggcatccccaactacaaccagaagttcaagggcagagccacactgaccgtgggcaagtctagcagcaccgcctacatggaactgcggagcctgacaagcgaggacagcgccgtgtacttctgcgccagaagaagaatcgcctacggctacgatgagggccacgccatggattattggggccagggaacaagcgtgaccgtgtctagt

SEQ ID NO: 57 - DNA, 840 bp, synthetic construct
  misc_feature 1..840: note = Codon-optimized FAP scFv VH/VL DNA
  source 1..840: mol_type = other DNA
atggactggatctggcggattctgtttctcgtgggagctgccacaggcgctcattctgctcagcctgcccaggttcagctgcagcagtctggacctgagctggttaagcctggcgcctccgtgaagatgagctgcaagaccagccggtacaccttcaccgagtacaccatccactgggtccgacagagccacggcaagagcctggaatggatcggcggcatcaaccccaacaacggcatccccaactacaaccagaagttcaagggcagagccacactgaccgtgggcaagtctagcagcaccgcctacatggaactgcggagcctgacaagcgaggacagcgccgtgtacttctgcgccagaagaagaatcgcctacggctacgatgagggccacgccatggattattggggccagggaacaagcgtgaccgtgtctagtggcggcggaggaagcggaggcggaggatctgggggcggaggctctggcggagggggatctgacatcgtgatgacacagagcccttctagcctggccgtgtccgtgggagagaaagtgaccatgagctgcaagagcagccagagcctgctgtactcccggaaccagaagaactacctggcctggttccagcagaagcccggccagtctcctaagctgctgatcttctgggccagcaccagagaaagcggcgtgcccgatagattcaccggcagcggctttggcaccgacttcaacctgacaatcagcagcgtgcaggccgaggacctggctgtgtacgattgccagcagtacttcagctaccctctgacctttggagccggcaccaagctggaactgaga

SEQ ID NO: 58 - protein, 280 aa, synthetic construct
  REGION 1..280: note = FAP scFvVL/VH protein
MDWIWRILFLVGAATGAHSAQPADIVMTQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELRGGGGSGGGGSGGGGSGGGGSQVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSS

SEQ ID NO: 59 - protein, 280 aa, synthetic construct
  REGION 1..280: note = FAP scFvVH/VL protein
MDWIWRILFLVGAATGAHSAQPAQVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSSGGGGSGGGGSGGGGSGGGGSDIVMTQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELR

SEQ ID NO: 60 - DNA, 1233 bp, synthetic construct
  misc_feature 1..1233: note = artificial sequence
  source 1..1233: mol_type = other DNA
atggactggatctggcggattctgtttctcgtgggagctgccacaggcgctcattctgctcagcctgccgacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagtcctcagggggcgggggaagtggtgggggcggcagcggcggagggggctcaggaggaggcggatcaggcggatcagaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagcgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 61 - DNA, 1230 bp, synthetic construct
  misc_feature 1..1230: note = artificial sequence
  source 1..1230: mol_type = other DNA
atggactggatctggcggattctgtttctcgtgggagctgccacaggcgctcattctgctcagcctgccgatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgtaagtaacttggaaactggcgtcccaagccgcttctctggatctggttcaggcaccgacttcactttcactatcagcagcctgcagcctgaagatatcgcaacctactattgccagcaggttgctaatgttcctctgactttcggccaaggcaccaaggtggagatcaagggcggcggaggaagcggaggcggaggatctgggggcggaggctctggcggagggggatctgaagttcagcttgtagaatctggaggtggattggttcaacctggtggctctcttcgcctgagttgtgcagcctctggttttactttctctagttactggatgcattgggttcgtcaggctcctgggaaaggcctggaatgggtttcagctattagttggagtggagatagtacttactacgcagacagtgtgaaaggtcgcttcaccatcagccgtgataattctaagaacactttgtacctgcaaatgaactccttgcgcgcagaagacacggctgtgtactattgtgcccgtgatcgctctgcgacttggtattatctggggcttggtttcgatgtatggggacaaggtaccctggtaacggtttctagcgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 62 - DNA, 1215 bp, synthetic construct
  misc_feature 1..1215: note = artificial sequence
  source 1..1215: mol_type = other DNA
atggactggatctggcggattctgtttctcgtgggagctgccacaggcgctcattctgctcagcctgccgatattcttcttactcaatctcccgttattttgtcagtatccccaggtgagcgagtcagcttctcttgtcgagcgtcacaatccattggcaccaacatacattggtaccaacagcgcaccaacgggtctccccggctcttgattaagtacgcatcagaaagtatttctgggatacccagtaggttctcagggagcgggagtggcactgactttaccctgtccataaacagcgttgagtctgaggacatcgcggactactattgtcagcagaacaacaattggccgaccacgtttggtgcgggaacaaaacttgaactcaaaggcggcggaggaagcggaggcggaggatctgggggcggaggctctggcggagggggatctcaggtgcagctcaaacagtcaggacctggcctcgttcagccaagccaatcactgagtataacgtgcacggtgagcggctttagcctgacaaactatggtgtccactgggtccgccaatctcctggaaaaggcttggagtggctcggtgttatctggtccggtggtaacacagactacaacacgccattcaccagtcgccttagtattaacaaggacaactccaagtctcaggttttctttaaaatgaactctctgcagtctaatgataccgcaatttactactgtgcgagggcactcacgtactatgactatgagttcgcgtattggggccaagggactctcgttactgtctcagcggcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 63 - DNA, 1230 bp, synthetic construct
  misc_feature 1..1230: note = artificial sequence
  source 1..1230: mol_type = other DNA
atggactggatctggcggattctgtttctcgtgggagctgccacaggcgctcattctgctcagcctgccgatgttgtaatgacgcagtcacccctgtcactcccggtcacacccggagaaccagcgtcaattagctgccgatctagccaaagtttgcttcattccaatggttacaattatctcgactggtacttgcagaaacccggccaatcccctcagctgctcatctaccttgggtctaatagggcatctggggttcccgataggttctctggctccgggagcggcaccgactttacgttgaaaatctctagggttgaggcggaagacgtaggcgtttactattgcatgcaggggacccactggccgctgaccttcggccagggcaccaaggttgaaataaaaggcggcggaggaagcggaggcggaggatctgggggcggaggctctggcggagggggatctcaggtacagctccaggaatcaggacccggtttggttaagccctccgggaccctttccctcacgtgtgcagtctcaggtgggtcaattagttcttccaattggtggtcttgggtgcggcaaccacctggtaaaggtctcgagtggataggggaaatttatcatagtggctccaccaattataacccctcactcaagtccagggttacgatatctgtggacaaaagtaaaaaccaattctccctcaaacttagtagtgtaacagcggcagacaccgcggtgtactactgcgcacggtggacaggccgaactgatgcctttgacatttggggacagggaactatggtgactgtgtcatccgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 64 - DNA, 1203 bp, synthetic construct
  misc_feature 1..1203: note = artificial sequence
  source 1..1203: mol_type = other DNA
atggactggatctggcgcatcctcttcctcgtcggcgctgctaccggcgctcattcggcccagccggccgacattcaaatgactcagtccccttccagcttgtcagcctcagtaggggaccgggtcacgatcacctgtcgagcgtctgagtcagtggataactacgggatttctttcatgaactggttccagcagaagcccggcaaagctcctaagctccttatatatgcagcctcaaatcaggggagcggtgttcctagtcgcttcagtggaagcggtagcggtacggactttacgttgacgataagtagccttcagccagatgactttgccacttattattgtcagcagtctaaggaagttccttggacgtttggccaaggaacgaaggtcgaaatcaaagggggagggggctcaggagggggcggcagtggtggtggaggctctcaagtccaactcgtacagtctggcgcggaggttaaaaagccgggaagctccgtgaaagtatcctgtaaggcaagcggatacacctttaccgattataacatgcactgggttaggcaggcgcccggccaaggtctggaatggatcggttatatttatccatacaacggtggtaccggctataatcagaagtttaagagtaaggctactattacagcggatgagtcaaccaatactgcatacatggagctctcctcactcaggagcgaagataccgcagtgtattactgtgcccgagggagaccagccatggactactggggtcagggtacccttgtgacagtatctagcgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 65 - DNA, 1215 bp, synthetic construct
  misc_feature 1..1215: note = artificial sequence
  source 1..1215: mol_type = other DNA
atggactggatctggcggattctgtttctcgtgggagctgccacaggcgctcattctgctcagcctgccaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctcagcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 66 - DNA, 1203 bp, synthetic construct; identical to SEQ ID NO: 64
  misc_feature 1..1203: note = artificial sequence
  source 1..1203: mol_type = other DNA

SEQ ID NO: 67 - DNA, 1596 bp, synthetic construct
  misc_feature 1..1596: note = artificial sequence
  source 1..1596: mol_type = other DNA
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccagcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 68 - DNA, 1215 bp, synthetic construct
  misc_feature 1..1215: note = artificial sequence
  source 1..1215: mol_type = other DNA
atggactggatctggcggattctgtttctcgtgggagctgccacaggcgctcattctgctcagcctgccgaagttcagcttgtagaatctggaggtggattggttcaacctggtggctctcttcgcctgagttgtgcagcctctggttttactttcaatagttacgctatgcattgggttcgtcaggctcctgggaaaggcctggaatgggtttcagctattagtggtaatggaggtagtactcgttacgcagacagtgtgaaaggtcgcttcaccatcagccgtgataattctaagaacactttgtacctgcaaatgaactccttgcgcgcagaagacacggctgtgtactattgtgcccgtgatcgctttcggaaggttcatggtttcgatgtatggggacaaggtaccctggtaacggtttctagcggaggtggtgggagtggtggaggcggctcgggtggaggtggttcaggaggaggcggagatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgcaagtaacttggaaactggcgtcccaagccgcttctctggatctggttcaggcaccgacttcactttcactatcagcagcctgcagcctgaagatatcgcaacctactattgccagcaggatgctacttttcctttgactttcggccaaggcaccaaggtggagatcaaggcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 69 - DNA, 1194 bp, synthetic construct
  misc_feature 1..1194: note = artificial sequence
  source 1..1194: mol_type = other DNA
atggactggatctggcggattctgtttctcgtgggagctgccacaggcgctcattctgctcagcctgccgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaggcggcggaggaagcggaggcggaggatctgggggcggaggctctgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaagcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 70 - DNA, 1206 bp, synthetic construct
  misc_feature 1..1206: note = artificial sequence
  source 1..1206: mol_type = other DNA
atggactggatctggcggattctgtttctcgtgggagctgccacaggcgctcattctgctcagcctgcccaggtgcaagtgaaagagtctggccctggactggtggccccaagccagtctctgagcatcacatgtaccgtgtccggcttcagcctgaccaactatggcgtgcactgggtccgacagcctccaggcaaaggactggaatggctgggagtgatttgggctggcggcagcaccaactacaacagcgccctgatgagccggctgagcatctccaaggacaacagcaagagccaggtgttcctgaagatgaacagcctgcagaccgacgacaccgccatgtactactgtgctagcagaggcggcaactacggctacgccctggattattggggccagggcacaagcgtgaccgtgtcatctggcggcggaggaagcggaggcggaggatctgggggcggaggctctggcggagggggatctagcatcgtgatgacccagactcctaagttcctgctggtgtctgccggcgacagagtgaccatcacctgtaaagccagccagagcgtgtccaacgacgtggcctggtatcagcagaagcctggacagagccccaagctgctgatctacagcgccagcaacagatacaccggcgtgcccgatagattcaccggctctggctacggcaccgacttcacctttaccatcagcaccgtgcaggccgaggatctggccgtgtacttctgccagcaagactacagctctctcggcggaggcaccaagctggaaatcaaagcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 71 - DNA, 1248 bp, synthetic construct
  misc_feature 1..1248: note = artificial sequence
  source 1..1248: mol_type = other DNA
atggactggatctggcggattctgtttctcgtgggagctgccacaggcgctcattctgctcagcctgcccaggttcagctgcagcagtctggacctgagctggttaagcctggcgcctccgtgaagatgagctgcaagaccagccggtacaccttcaccgagtacaccatccactgggtccgacagagccacggcaagagcctggaatggatcggcggcatcaaccccaacaacggcatccccaactacaaccagaagttcaagggcagagccacactgaccgtgggcaagtctagcagcaccgcctacatggaactgcggagcctgacaagcgaggacagcgccgtgtacttctgcgccagaagaagaatcgcctacggctacgatgagggccacgccatggattattggggccagggaacaagcgtgaccgtgtctagtggcggcggaggaagcggaggcggaggatctgggggcggaggctctggcggagggggatctgacatcgtgatgacacagagcccttctagcctggccgtgtccgtgggagagaaagtgaccatgagctgcaagagcagccagagcctgctgtactcccggaaccagaagaactacctggcctggttccagcagaagcccggccagtctcctaagctgctgatcttctgggccagcaccagagaaagcggcgtgcccgatagattcaccggcagcggctttggcaccgacttcaacctgacaatcagcagcgtgcaggccgaggacctggctgtgtacgattgccagcagtacttcagctaccctctgacctttggagccggcaccaagctggaactgagagcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 72 - DNA, 510 bp, synthetic construct
  misc_feature 1..510: note = artificial sequence
  source 1..510: mol_type = other DNA
atgcgcatcagcaagccccacctgcgcagcatcagcatccagtgctacctgtgcctgctgctgaacagccacttcctgaccgaggccggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagcggctccgagaaggacgagctgtaa

SEQ ID NO: 73 - protein, 169 aa, synthetic construct
  REGION 1..169: note = artificial sequence
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSEKDEL

SEQ ID NO: 74 - DNA, 1215 bp, synthetic construct
  misc_feature 1..1215: note = artificial sequence
  source 1..1215: mol_type = other DNA
atggactggatctggcgcatcctcttcctcgtcggcgctgctaccggcgctcattcggcccagccggccatggcgcaagtaaaactccaagaatctggggcggagctggtgaaaccgggggcgtctgtgaagatgagctgtaaagcatcaggctacaccttcacctcctataatatgcactgggtgaaacaaacacccggacagggcctcgaatggattggtgccatctatcctggaaatggtgatacctcatataatcagaagtttaagggcaaggctacgcttactgcggataaaagctcttccactgcttacatgcaactgagcagtctcacttcagaggactcagccgattattattgtgcccgcagcaactactatggtagttcatactggtttttcgacgtttgggggcaaggtaccaccgtcacggtttcttctggtgggggcggaagcgggggtggaggatctgggggcggtggttcagacattgaactcacccagagccctactattctgagcgcgtctccaggtgaaaaagttacgatgacgtgcagagcatcaagtagtgtgaattatatggattggtatcaaaagaagccaggctcatccccaaaaccgtggatctatgcaactagcaacctcgcgtcaggggtgccagcaaggttttccggaagtggttctggcacatcttatagtctcaccatttcccgagtggaggctgaggatgcggccacttattactgccagcaatggtcattcaatcccccaacatttggtggcggaacaaaactcgaaattaaacgggcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 75 - DNA, 1248 bp, synthetic construct
  misc_feature 1..1248: note = artificial sequence
  source 1..1248: mol_type = other DNA
atggactggatctggcgcatcctcttcctcgtcggcgctgctaccggcgctcattcggcccagccggccgatatcgagctcacccaatctccaaaattcatgtccacatcagtaggagacagggtcagcgtcacctgcaaggccagtcagaatgtggatactaatgtagcgtggtatcaacaaaaaccagggcaatctcctgaaccactgcttttctcggcatcctaccgttacactggagtccctgatcgcttcacaggcagtggatctgggacagatttcactctcaccatcagcaatgtgcagtctgaagacttggcagagtatttctgtcagcaatataacagctatcctctgacgttcggtggcggcaccaagctggaaatcaaacgggctgccgcagaaggtggaggcggttcaggtggcggaggttccggcggaggtggctctggcggtggcggatcggccatggcccaggtgaagctgcagcagtcaggagggggcttggtgcaacctggaggctccatgaaactctcctgtgttgtctctggattcactttcagtaattactggatgaactgggtccgccagtctccagagaaggggcttgagtggattgcagaaattagattgaaatccaataattttggaagatattatgcggagtctgtgaaagggaggttcaccatctcaagagatgattccaaaagtagtgcctacctgcaaatgatcaacctaagagctgaagatactggcatttattactgtaccagttatggtaactacgttgggcactattttgaccactggggccaagggaccacggtcaccgtatcgagtgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 76 - DNA, 6787 bp, synthetic construct
  misc_feature 1..6787: note = artificial sequence
  source 1..6787: mol_type = other DNA
tgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgggatccgctgaccaaaagagcaccaaaggcgccctgaccttcagcccctacctgcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgataatacgactcactatagggagacccaagctggaattcgccaccatggactggatctggcggattctgtttctcgtgggagctgccacaggcgctcattctgctcagcctgccgatgttgtaatgacgcagtcacccctgtcactcccggtcacacccggagaaccagcgtcaattagctgccgatctagccaaagtttgcttcattccaatggttacaattatctcgactggtacttgcagaaacccggccaatcccctcagctgctcatctaccttgggtctaatagggcatctggggttcccgataggttctctggctccgggagcggcaccgactttacgttgaaaatctctagggttgaggcggaagacgtaggcgtttactattgcatgcaggggacccactggccgctgaccttcggccagggcaccaaggttgaaataaaaggcggcggaggaagcggaggcggaggatctgggggcggaggctctggcggagggggatctcaggtacagctccaggaatcaggacccggtttggttaagccctccgggaccctttccctcacgtgtgcagtctcaggtgggtcaattagttcttccaattggtggtcttgggtgcggcaaccacctggtaaaggtctcgagtggataggggaaatttatcatagtggctccaccaattataacccctcactcaagtccagggttacgatatctgtggacaaaagtaaaaaccaattctccctcaaacttagtagtgtaacagcggcagacaccgcggtgtactactgcgcacggtggacaggccgaactgatgcctttgacatttggggacagggaactatggtgactgtgtcatccgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccagggatctggagctactaacttcagcctgctgaagcaggctggagacgtggaggagaaccctggacctatgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtgaaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgccaccatgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtgagcggccgcccgctgatcagcctcgaacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtgcggtgggctctatggcttctgaggcggaaagaaccagctggggctctagggggtatccccggatcctgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaa